SEQUENCE LISTING (46 sequences)

SEQ ID NO: 1 - DNA, 8685 bp, synthetic construct
  source 1..8685: mol_type = other DNA
ctgacgcgccctgtagcggcgcattaagcgcggcgggtgtggtgggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcgcaggaccagatcaatacagggactccatctcagccagccctcagggagacagacagcccggccatggggtccccagacctgattatcccctcgacttctcctgctctgacttcccagagtcccacgtgcagcggggtgccatgtgctcacagagggcccagagccctggactcagatgctgcctgctctttgggactcactcttgcccagggcccctctacgccagtatttcccagcatgctccactctagcctccttgctgcatccattcatcaaaatccacccacctcactgcccggctagcaacaccatagccatggcaacgtgggatccgagcctcgtctgcaacctatacggcagcttgtggtcatgccggatccttaacccactgagcgaggccagggattgaacccacatcctcatggacgctagctgggttcgttaaccactgagccacgacaggaactccccagtccttcatttttaaaatttaaaaacagacctcgaaagggttcacctcatgttggaggcagagctggagcaaggaaccaggtccctcttaaggttactagccttcctctggagtgcgcagcgggtgcctgatgaaatgggttgccagcgcaatgtgcagtgtgtccgtgggaaacagaggctgcaggcgctacacagcaaatgtggaaagaagagagatgagtgtgggctctgcagttcctttgtgacgtgctctctgctctttgcatatctgtccccttgggccaggcttccatcctgaagggtgtgaccatagtgtgtcttgtttcttgtcaccgcagagaacggaaggctcccttgatgggactactagtgagggcagaggaagtcttctaacatgcggtgacgtggaggagaatcccggaccggggggaccagagccagcgaagtctgctcccgccccgaaaaagggctccaagaaggcggtgactaaggcgcagaagaaagacggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccataacttcgtataatgtatgctatacgaagttatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctcgtgtcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccagatcgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcntgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctggtcgagataacttcgtataatgtatgctatacgaagttatggcgcgccatgggacttaggaccgcagagcagacgctcctctccgggaggacaccgcattctgagaagagcggacagtccttgtgttccaagagttttgcgcacttatttatgaacctgccctgctcccactgaatgtagcagttcctcaggccaagtcaccacttcttcaatccatcttagctacatttatgctgggtataaagagagaacggtggggctggttaatttcccacccggcccgcctcccaccgcattggagcatccctgggagtggagagcagctgccggaggttcagagtaaactggccattgggacactttgaaacttgaatattttgtcttgtacagagataaagaacttttccaagtaccctgatacacggaaaccagtatctgatttgtagtcaatcacttctagcgaatggcctggcataggggctagcttttctctttgcctgtggtccttctgaggtctgtggttctgggcagtccttgtttcttggaagtaccgggcatggacttgacagctgcaccctgtcccctctgtgacctcgcctgccacaaatgcgaaaacagagctgctcgggcacaacccctttgagatgccgacttgaagacaaggctccttctgggcgatgcaccaaattgaaaatgaagttggacgaagcacagatgttcttaagctgtttttctctcccctttctttctctttctctacctgctgacggctgaaggatgggaagatggtagcctacagcaaatactttttttaatagaaaaaggaaatgcatatttttcttactaatttttaaaaatgtattccttgctaaaggaatggtctcccgagatcttccgacgtttctgatggtgtcttgagtggatggacggtgggctgcagtgggatcgccccagtgagcacacctaaccgaaatcacttcccctgtcctcctcgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttagagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 9175 bp, synthetic construct
  source 1..9175: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccacaacctccccttctacgagcggctcggcttcaccgtcaccgccgacgtcgaggtgcccgaaggaccgcgcacctggtgcatgacccgcaagcccggtgcctgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 20 bp, synthetic construct
  source 1..20: mol_type = other DNA
tgaacctcagctacaaacag

SEQ ID NO: 4 - DNA, 20 bp, synthetic construct
  source 1..20: mol_type = other DNA
cctcacttcactgcactgta

SEQ ID NO: 5 - DNA, 24 bp, synthetic construct
  source 1..24: mol_type = other DNA
ttcacatgtcccagcactaccaga

SEQ ID NO: 6 - DNA, 23 bp, synthetic construct
  source 1..23: mol_type = other DNA
ttcactacagtcgctacatcacc

SEQ ID NO: 7 - DNA, 20 bp, synthetic construct
  source 1..20: mol_type = other DNA
acatctcgcccttcagccac

SEQ ID NO: 8 - DNA, 20 bp, synthetic construct
  source 1..20: mol_type = other DNA
aggatgtggatgctgcctac

SEQ ID NO: 9 - DNA, 22 bp, synthetic construct
  source 1..22: mol_type = other DNA
caggtggtggagaaagttgaga

SEQ ID NO: 10 - DNA, 30 bp, synthetic construct
  source 1..30: mol_type = other DNA
acaggatccatgttgtggaaaataaccgat

SEQ ID NO: 11 - DNA, 22 bp, synthetic construct
  source 1..22: mol_type = other DNA
agaaacgaagatccccagatga

SEQ ID NO: 12 - DNA, 22 bp, synthetic construct
  source 1..22: mol_type = other DNA
tccattccgctcctgatgtgca

SEQ ID NO: 13 - DNA, 19 bp, synthetic construct
  source 1..19: mol_type = other DNA
accctggctgtggagaagg

SEQ ID NO: 14 - DNA, 22 bp, synthetic construct
  source 1..22: mol_type = other DNA
gttaatgacatcaaaggcaccc

SEQ ID NO: 15 - DNA, 20 bp, synthetic construct
  source 1..20: mol_type = other DNA
gctgaccttcacactcccca

SEQ ID NO: 16 - DNA, 20 bp, synthetic construct
  source 1..20: mol_type = other DNA
cagatacctggagaacggga

SEQ ID NO: 17 - DNA, 22 bp, synthetic construct
  source 1..22: mol_type = other DNA
tggcacccatttacacctacac

SEQ ID NO: 18 - DNA, 23 bp, synthetic construct
  source 1..23: mol_type = other DNA
ctcaacacaataaaggagctggg

SEQ ID NO: 19 - DNA, 21 bp, synthetic construct
  source 1..21: mol_type = other DNA
tttctcaggcccaattttggt

SEQ ID NO: 20 - DNA, 21 bp, synthetic construct
  source 1..21: mol_type = other DNA
ggagcacacgctagttcagaa

SEQ ID NO: 21 - DNA, 20 bp, synthetic construct
  source 1..20: mol_type = other DNA
gtcaggggtggttattgcat

SEQ ID NO: 22 - DNA, 17 bp, synthetic construct
  source 1..17: mol_type = other DNA
tccagtcacggtcagca

SEQ ID NO: 23 - DNA, 22 bp, synthetic construct
  source 1..22: mol_type = other DNA
ctcttctgcctgctgcactttg

SEQ ID NO: 24 - DNA, 20 bp, synthetic construct
  source 1..20: mol_type = other DNA
caaattccatggcaccgtca

SEQ ID NO: 25 - DNA, 20 bp, synthetic construct
  source 1..20: mol_type = other DNA
tggtggtaggaagagtaaag

SEQ ID NO: 26 - DNA, 20 bp, synthetic construct
  source 1..20: mol_type = other DNA
caggttttctttccctagct

SEQ ID NO: 27 - DNA, 26 bp, synthetic construct
  source 1..26: mol_type = other DNA
tcacatgtgtgagaggggcagtgtgc

SEQ ID NO: 28 - DNA, 22 bp, synthetic construct
  source 1..22: mol_type = other DNA
ttgattttcctctcctttgctc

SEQ ID NO: 29 - DNA, 21 bp, synthetic construct
  source 1..21: mol_type = other DNA
catggcggtgaccatgctgga

SEQ ID NO: 30 - DNA, 20 bp, synthetic construct
  source 1..20: mol_type = other DNA
caccacagatgtgtcggaga

SEQ ID NO: 31 - DNA, 22 bp, synthetic construct
  source 1..22: mol_type = other DNA
gtgcttgagcttgtggatgaag

SEQ ID NO: 32 - DNA, 30 bp, synthetic construct
  source 1..30: mol_type = other DNA
atactcgagtttcctgtgtttctccatttt

SEQ ID NO: 33 - DNA, 22 bp, synthetic construct
  source 1..22: mol_type = other DNA
ctgttgctgttgcctgtacgtt

SEQ ID NO: 34 - DNA, 23 bp, synthetic construct
  source 1..23: mol_type = other DNA
cgtcttcttggaccttagtggag

SEQ ID NO: 35 - DNA, 18 bp, synthetic construct
  source 1..18: mol_type = other DNA
atggactcgaagcgcaca

SEQ ID NO: 36 - DNA, 21 bp, synthetic construct
  source 1..21: mol_type = other DNA
ccccatctcaacaggaaaacc

SEQ ID NO: 37 - DNA, 21 bp, synthetic construct
  source 1..21: mol_type = other DNA
caaaggtgaagtcctgctccc

SEQ ID NO: 38 - DNA, 20 bp, synthetic construct
  source 1..20: mol_type = other DNA
cagtatgatctccgcagggt

SEQ ID NO: 39 - DNA, 22 bp, synthetic construct
  source 1..22: mol_type = other DNA
atgtcaggagaggccccataga

SEQ ID NO: 40 - DNA, 23 bp, synthetic construct
  source 1..23: mol_type = other DNA
aaaggggatacaggtttttccac

SEQ ID NO: 41 - DNA, 21 bp, synthetic construct
  source 1..21: mol_type = other DNA
gctgtcggaggtatatggtgg

SEQ ID NO: 42 - DNA, 21 bp, synthetic construct
  source 1..21: mol_type = other DNA
ggtctctattgaggggaaggt

SEQ ID NO: 43 - DNA, 20 bp, synthetic construct
  source 1..20: mol_type = other DNA
agtgaggaacaagccagagc

SEQ ID NO: 44 - DNA, 21 bp, synthetic construct
  source 1..21: mol_type = other DNA
cagcctcagagtgtttcttct

SEQ ID NO: 45 - DNA, 22 bp, synthetic construct
  source 1..22: mol_type = other DNA
atgggctacaggcttgtcactc

SEQ ID NO: 46 - DNA, 20 bp, synthetic construct
  source 1..20: mol_type = other DNA
atcgccccacttgattttgg

(19) United States
(12) Patent Application Publication
(10) Pub. No.: US 2026/0146991 A1
(43) Pub. Date: May 28, 2026

(54) COMPOSITION AND METHODS OF AGING-RELATED AGENT SCREENING AND TARGET ANALYSIS

(71) Applicant: Centre for Translational Stem Cell Biology Limited, Hong Kong (CN)

(72) Inventors: Pengtao LIU, Hong Kong (CN); Zhen FENG, Hong Kong (CN); Degong RUAN, Hong Kong (CN); Fang LIU, Hong Kong (CN)

(21) Appl. No.: 19/114,569

(22) PCT Filed: Sep. 25, 2023
(86) PCT No.: PCT/CN2023/121168

§ 371 Date: Mar. 24, 2025

(30) Foreign Application Priority Data
Sep. 25, 2022 (WO) PCT/CN2022/121158

(51) Int. Cl. G01N 33/50; C12Q 1/6897

(52) U.S. Cl. CPC G01N 33/5023; C12Q 1/6897; G01N 33/5076

(57) ABSTRACT

Provided is a mammalian derived cell model which can be used in anti-aging drugs. The mammalian-derived extracembryonic trophoblast cell model is an early mammalian-derived extracembryonic stage. The change model of differentiation of cells into exoembryonic trophoblastic cells/organoids and the aging evaluation index system can be used various anti-aging research and target testing, anti-aging drug detection, screening, identification and innovative drugs/natural products Research and development of chemicals, and/or health foods and additives. It is helpful to screen and identify many resistances Candidate targets for aging can be further mechanized and functionally validated for future therapeutic use. The Screening of anti-aging drugs or natural products will produce huge economic and social benefits.

### CROSS-REFERENCE TO RELATED APPLICATIONS

This application is a U.S. national stage application under 35 U.S.C. § 371 of International Application No. PCT/CN2023/121168, filed internationally on Sep. 25, 2023, which claims priority to PCT application No. PCT/CN2022/121158, filed Sep. 25, 2022, the content of each of which is hereby expressly incorporated by reference in its entirety.

### REFERENCE TO AN ELECTRONIC SEQUENCE LISTING

The contents of the electronic sequence listing (275162000200SEQLIST.xml; Size: 59,502 bytes; and Date of Creation: Mar. 20, 2025) is herein incorporated by reference in its entirety.

### FIELD

The present invention relates to the field of using cultured cells, in particular mammalian cells and its application in assessing mammalian cellular aging, evaluating and screening for potential anti-aging agents, and identifying target genes involved in aging.

### BACKGROUND OF THE INVENTION

Aging is a natural process that occurs in various organisms and is affected by factors including environmental, genetic, and epigenetic factors, resulting in damages to cells and tissues. Various human diseases involve aging, such as diabetes, neurodegenerative diseases, heart diseases, vascular disease, and cancer etc. Various other processes of aging can be found in various other mammals.

However, studying aging in mammals is time-consuming due to the long life span. Other organisms have been used to study aging (as summarized in Table 1). Among the earliest and simplest model organisms used to study aging is the yeast, Saccharomyces cerevisiae. With a lifespan ranging from a few days to several weeks, the yeast's replicative and chronological lifespans have been monitored using two different models, each revealing significant insights into aging pathways and potential interventions. However, the yeast model is not without limitations. Although 90% of the yeast genes have been characterized, only 30% of the genome is conserved to humans. Another challenge is pleiotropic drug resistance (PDR), which necessitates further validation of optimal dosage of a bioactive compound in animals models.

The nematode Caenorhabditis elegans has also been a popular model organism in aging research. Its short lifespan and high genetic homology (60-80%) with humans make it a suitable candidate for high-throughput screening for anti-aging compounds. However, C. elegans lacks critical epigenetic changes that occur in human aging processes such as DNA methylation.

The fruit fly, Drosophila melanogaster, another model organism in aging research, offers insights into aging at the level of whole-organism physiology and behavior. Despite the advantages this model offers, such as low cost and the case of generating large populations, its small size and unclear death reasons present challenges to aging research. Additionally, many drugs are not included in the drug library when performing high-throughput drug screening in Drosophila, mainly because the solvent commonly used for drugs is DMSO, which is cytotoxic to Drosophila.

The mouse model, despite its longer lifespan and costs, offers a degree of genetic manageability and a large amount of available baseline phenotypic data, making it a powerful tool in aging research. Yet, its different genetic background and physiological aspects compared to humans pose some limitations.

There have also been attempts to use non-canonical models like the African turquoise killifish, birds, non-human primates, and dogs, each offering unique advantages. Accompanied with their rapid reproduction, killifish has an accelerated aging process among vertebrates of 4-6 months. Avian models have provided multiple anti-aging insights given their distinguished resistance to detrimental factors like oxidative stress. Primates are evolutionarily closer to humans and have allowed testing of interventions and compounds. Companion dogs share an environment with humans and their genetics is more tractable than that of primates. Despite these features, these models still present tremendous challenges, including limited reference and homology to human, cost, time, and ethical concerns.

Various other model animals have also been used in studies of aging (Table 1), but the implications are limited for a number of reasons. The model animals are distinct from humans in the absorption, distribution, metabolism, and secretion functions, and their proteomes and genomes. Therefore, many experimental results cannot be readily applied to human.

In recent years, human cell lines, particularly fibroblasts as an example from primary cells, and induced pluripotent stem cells (iPSCs) have gained attention as models for studying aging due to their relevance to human aging and diseases. Cellular aging in vitro was demonstrated to share numerous features with in vivo aging, such as loss of division potential, chromatin alterations, etc. Readouts for in vitro aging has also significantly improved over the past few years, from the decline in cellular replication of which the accuracy varies for different measures, to more systematic, transcriptome-based approaches like CultureAge, scAge, and AgeScore. Nevertheless, current applications were often directed to one aspect of aging, like skin aging and vascular aging, instead of human aging in general. Mesenchymal stem cells (MSC) is another type of cells that have been explored to be used in aging research. However, MSC differentiation phenotypes only partially overlap with natural aging.

The use of accelerated aging models offers certain advantages over natural aging models, such as time efficiency and the ability to study age-related changes in a controlled environment. A few artificially generate mouse lines are the most popular options for accelerated aging models, while the differences from human in various aspects including physiological, metabolic, and environmental factors are yet to be tackled.

While a number of promising drugs have been screened using the above models, drug screening using only these models could leave some side effects unidentified. For example, aspirin, ibuprofen and celecoxib are drugs that have been shown to extend lifespan in some model organisms, but not in humans. Aspirin, previously demonstrated in C. elegans, Drosophila, and male mice, has been shown to increase the risk of gastrointestinal bleeding while ibuprofen and celecoxib, which were studied in yeast, worms and flies, have been shown to have side effects like major bleeding events and increase in all-cause mortality in humans.

TABLE 1
Model organisms and their use in aging studies

| | |
|---|---|
| Organism/Similarity | Aging-related phenotypes |
| to human | |
| genome/Lifespan | |
| S. cerevisiae; | Decelerated population survival and loss of viability; |
| 30% yeast genome; | Unambiguous phenotypes (e.g. certain pathways or genes). |
| a few days to a few | |
| weeks | |
| C. elegans, | Organism-level: |
| 75% nematode | |
| genes, | |
| 2.5-4 days | Reduced activity, lack of coordination in movements, dormancy, |
| | cessation of reproductive processes, and accumulation of |
| | fluorescent deposits within cells have been documented; |
| | Decline in tissue integrity, decreased mobility, impaired learning |
| | and memory, and compromised immune function; |
| | Decline in reproductive capabilities, including delayed |
| | reproduction, reduced oocyte size and quality; |
| | Loss of synaptic integrity: touch receptor neurons exhibit structural |
| | changes such as blebbing and branching; |
| | Impaired motility and abnormal appearance due to the loss of |
| | muscular integrity, decreased sarcomere density, and irregular |
| | orientations |
| | Cellular-level: |
| | Nuclear integrity diminishes, and the relative size of nucleoli |
| | increases; |
| | Increased fragmentation and impaired fusion of mitochondria; |
| | Accumulation of misfolded proteins; |
| | Decrease in quality of RNA control mechanisms, including non- |
| | sense-mediated mRNA decay (NMD); |
| | Decline in mRNA splicing fidelity, including Elevated levels of |
| | introns and unannotated regions in the mRNAs |
| | Protein-level: those involved in nucleosome assembly, ER nuclear |
| | signalling, and the response to unfolded proteins increase, while |
| | those involved in metabolism (fatty acid, carbohydrate, and amino |
| | acid) decreases |
| D. melanogaster, | Decrease in resting metabolic rate and reduced synthesis of proteins |
| 60% fly genome, | and fats. |
| max 15 weeks | Behavioural changes, including decreased feeding, courtship, and |
| | exploration, as well as increased sleep fragmentation. |
| | decline in stress resistance. |
| | Diminished reproductive capacity, with reduced egg laying and |
| | hatching success, decreased production of sperm and accessory |
| | fluid, and decreased success in sperm competition. |
| | Impaired neuronal functions, e.g. learning and memory. |
| | Hampered physical activity, with impaired negative geotaxis |
| | (response to gravity), reduced voluntary flying and walking. |
| | Immune system dysfunctions. |
| | Progressive dysplasia, with a decrease in the barrier function of the |
| | gut; |
| | Compromised cardiac function. |
| Mouse, | Loss of proliferation potential; |
| 99%, | Alterations in chromatin structure; |
| 1-1.5 years | Impaired ability to migrate; |
| | Increase in cell size, volume, and protein content, along with a |
| | decreased response to growth factors stimulating cell division. |
| Human cell lines, | Distinct morphological changes during replicative senescence, |
| 100%, | including enlarged cell size, nuclear size, nucleolar size, |
| Weeks to months | multinucleated cells, prominent Golgi apparatus, increased vacuoles |
| | in the endoplasmic reticulum and cytoplasm, elevated cytoplasmic |
| | microfilaments, and larger lysosomal bodies; |
| | Heightened sensitivity to cell contact and reduced cell density |
| | during harvesting and saturation; |
| | Decrease in macromolecule synthesis, while increase in the |
| | intracellular content of RNA and proteins, accompanied by an |
| | enlargement of cells and nuclei, the presence of inclusion bodies in |
| | late-passage cells, and a potential disruption of protein degradation |
| | through proteasome-mediated pathways.; |
| | Decline in RNA turnover and the decoupling of cell growth from |
| | cell division (potentially causing a block in late G1 phase). |

Chinese patent document CN107858330B discloses a method of screening anti-aging drugs using a mouse bone marrow hematopoietic stem/progenitor cell model in vitro. The method comprises isolating hematopoietic stem/progenitor cells from mouse bone marrow, and culturing the cells for 5-14 days in stem cell culture medium with IL3, IL6 and SCF. A large number of senescent hematopoietic stem/progenitor cells could be rapidly obtained by this method, and it is reported that they can be used to screen anti-aging drugs. Since this method utilizes mouse bone marrow hematopoietic stem/progenitor cells to screen of anti-aging drugs, it is difficult to be adapted to other mammals and human. Additionally, the method is complicated to carry out and the evaluation system is inefficient, thus the screening results are uncertain and cannot be easily applied widely. Therefore, there still exists a need to establish a more reliable, relevant, and human-specific system for cellular aging evaluation, and method for assessing aging, evaluating and screening for anti-aging agents, and identifying candidate genes involved in aging.

### BRIEF SUMMARY OF THE INVENTION

The terms “invention,” “the invention,” “this invention” and “the present invention,” as used in this disclosure, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present disclosure and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present disclosure describes and refers to various embodiments of the invention. No particular embodiment is intended to define the scope of the invention. Rather, the embodiments merely provide non-limiting examples of various methods, compositions, kits, systems etc. that are at least included within the scope of the invention. Some embodiments of the present invention are summarized below, while others are described and shown elsewhere in the present disclosure.

In some embodiments, provided herein is a mammalian cellular aging evaluation system comprising a totipotent or pluripotent stem cell or an early extraembryonic cell, wherein the totipotent or pluripotent stem cell or early extraembryonic cell optionally comprises a reporter molecule or a heterologous nucleic acid encoding a reporter molecule that is indicative of senescence or differentiation of the totipotent or pluripotent stem cell or early extraembryonic cell towards a trophoblast or syncytiotrophoblast (“STB”).

In some embodiments, the totipotent or pluripotent stem cell or early extraembryonic cell is naturally occurring. In some embodiments, the totipotent or pluripotent stem cell or early extraembryonic cell is genetically modified.

In any of the embodiments herein, the totipotent or pluripotent stem cell or early extraembryonic cell can comprise a reporter molecule. In some embodiments, the reporter molecule is selected from the group consisting of: a physically activated molecule and a chemically activated molecule.

In any of the embodiments herein, the totipotent or pluripotent stem cell or early extraembryonic cell can comprise the heterologous nucleic acid. In some embodiments, the heterologous nucleic acid is integrated into the genome of the totipotent or pluripotent stem cell or early extraembryonic cell. In some embodiments, the heterologous nucleic acid is under the control of a promoter of an endogenous biomarker gene encoding a biomarker indicative of senescence or differentiation of the totipotent or pluripotent stem cell or early extraembryonic cell towards a trophoblast or STB. In some embodiments, the heterologous nucleic acid is introduced into the totipotent or pluripotent stem cell or early extraembryonic cell by a gene editing tool.

In any of the embodiments herein, the reporter molecule can be selected from the group consisting of Green Fluorescent Protein (GFP), Red Fluorescent Protein (RFP), Yellow Fluorescent Protein (YFP), mCherry, tdTomato, photoconvertible fluorescent proteins, bioluminescence, enzyme assay, antibody-based assays, chloramphenicol acetyltransferase, and biosensors.

In any of the embodiments herein, the system can comprise an early extraembryonic cell. In some embodiments, the early extraembryonic cell is a trophoblast stem cell (“TSC”) or a trophoblast progenitor cell (“TPC”). In some embodiments, the early extraembryonic cell is a TSC. In any of the embodiments herein, the early extraembryonic cell can be derived from a totipotent stem cell, a pluripotent stem cell, an embryo tissue, or a placenta tissue. In any of the embodiments herein, the early extraembryonic cell can be derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

In any of the embodiments herein, the endogenous biomarker can be selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. and stem cell exhaustion.

In some embodiments, the endogenous biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, and SIRT family members.

In any of the embodiments herein, the system can comprise a totipotent or pluripotent stem cell. In some embodiments, the system comprises a totipotent stem cell. In some embodiments, the system comprises a pluripotent stem cell. In any of the embodiments herein, the totipotent or pluripotent stem cell or early extraembryonic cell can be derived from a human, a pig, a cow, a mouse, a rat, a bat, a rabbit, a dog, a cat, and a sheep.

In some embodiments, provided herein is a method of assessing an aging process of a mammalian cell, comprising subjecting a totipotent or pluripotent stem cell or an early extraembryonic cell to a condition that allows differentiation of the totipotent or pluripotent stem cell or extraembryonic cell towards a trophoblast or STB, and determining one or more characteristics of differentiation of the totipotent or pluripotent stem cell or the early extraembryonic cell.

In some embodiments, provided herein is a method of evaluating anti-aging function of a candidate agent, comprising: 1) subjecting a totipotent or pluripotent stem cell or an early extraembryonic cell to a condition that allows differentiation of the totipotent or pluripotent stem cell or early extraembryonic cell towards a trophoblast, specifically STB; 2) contacting the totipotent or pluripotent stem cell or early extraembryonic cell with the candidate agent before, during, or after subjecting the totipotent or pluripotent stem cell or early extraembryonic cell to a condition that allows the differentiation; and 3) assessing change of one or more characteristics of differentiation of the totipotent or pluripotent stem cell or early extraembryonic cell relative to a totipotent or pluripotent stem cell or an early extraembryonic cell without contacting with the candidate agent.

In some embodiments, evaluating anti-aging function of the candidate agent comprises assessing whether the candidate agent has anti-aging function. In some embodiments, evaluating anti-aging function of the candidate agent comprises determining an effective concentration of the candidate agent having an anti-aging function.

In some embodiments, provided herein is a method of screening for a candidate agent having an anti-aging function, comprising: 1) evaluating the anti-aging function of a plurality of candidate agents according to the method of any of the embodiments herein; and 2) identifying the candidate agent having an anti-aging function based on the ability of the candidate agent to cause change of one or more characteristics of differentiation of the totipotent or pluripotent stem cell or early extraembryonic cell to a trophoblast or STB relative to a totipotent or pluripotent stem cell or early extraembryonic cell without the candidate agent.

In any of the embodiments herein, the candidate agent can be selected from the group consisting of: an antibody, a virus, a virus-like, a small molecule, a peptide, a polypeptide, a DNA, an mRNA, a guide RNA, a microRNA, an RNAi, a LncRNA, an siRNA molecule, and an antisense RNA. In any of the embodiments herein, the candidate agent can be a naturally occurring substance. In any of the embodiments herein, the candidate agent can be a nutraceutical.

In some embodiments, provided herein is a method of identifying a candidate gene involved in an aging process, comprising: 1) subjecting a plurality of totipotent or pluripotent stem cells or early extraembryonic cells to a condition that allows differentiation of the plurality of totipotent or pluripotent stem cells or early extraembryonic cells towards trophoblasts or STBs, wherein each of the plurality of totipotent or pluripotent stem cells or early extraembryonic cells comprises an alteration in a candidate gene relative to a wildtype totipotent or pluripotent stem cell or early extraembryonic cell, wherein at least two of the plurality of totipotent or pluripotent stem cells or early extraembryonic cells contain different alterations; 2) determining one or more characteristics of differentiation of the plurality of totipotent or pluripotent stem cells or early extraembryonic cells; and 3) identifying the candidate gene involved in an aging process based on the ability of the alteration in the candidate gene to cause change of one or more characteristics of differentiation of the plurality of totipotent or pluripotent stem cell or early extraembryonic cell relative to a totipotent or pluripotent stem cell or an early extraembryonic cell without the corresponding alternation.

In any of the embodiments herein, the totipotent or pluripotent stem cell or early extraembryonic cell can be naturally occurring. In any of the embodiments herein, the totipotent or pluripotent stem cell or early extraembryonic cell can be genetically modified.

In any of the embodiments herein, the totipotent or pluripotent stem cell or early extraembryonic cell can comprise a reporter molecule that is indicative of differentiation of the totipotent or pluripotent stem cell or early extraembryonic cell towards an STB. In some embodiments, the reporter molecule is selected from the group consisting of: a physically activated molecule and a chemically activated molecule.

In any of the embodiments herein, the totipotent or pluripotent stem cell or early extraembryonic cell can comprise a heterologous nucleic acid encoding a reporter molecule that is indicative of differentiation of the totipotent or pluripotent stem cell or early extraembryonic cell towards a trophoblast or STB. In some embodiments, the heterologous nucleic acid is integrated into the genome of the totipotent or pluripotent stem cell or early extraembryonic cell. In some embodiments, the heterologous nucleic acid is under the control of a promoter of an endogenous biomarker gene encoding a biomarker indicative of differentiation of the totipotent or pluripotent stem cell or early extraembryonic cell towards an STB. In any of the embodiments herein, the heterologous nucleic acid can be introduced into the totipotent or pluripotent stem cell or early extraembryonic cell by a gene editing tool. In any of the embodiments herein, the reporter molecule can be selected from the group consisting of Green Fluorescent Protein (GFP), Red Fluorescent Protein (RFP), Yellow Fluorescent Protein (YFP), mCherry, and tdTomato.

In any of the embodiments herein, the STB can be an early STB, a late STB, or a mature and aged STB.

In any of the embodiments herein, the one or more characteristics of differentiation can comprise: 1) presence or absence of a biomarker associated with differentiation of the totipotent or pluripotent stem cell or early extraembryonic cell; 2) level of a biomarker associated with differentiation of the totipotent or pluripotent stem cell or early extraembryonic cell; 3) secretion of a biomarker associated with differentiation of the totipotent or pluripotent stem cell or early extraembryonic cell; 4) cell morphology; 5) rate of change to a differentiated state; 6) property of a cellular organelle; and 7) number of nuclei in the cell; and 8) the presence or absence of a reporter molecule.

In some embodiments, the one or more characteristics of differentiation comprises property of a cellular organelle. In some embodiments, the cellular organelle is selected from the group consisting of: mitochondria, proteosome, endoplasmic reticulum, Golgi apparatus, and nuclear envelope. In some embodiments, the property of the cellular organelle comprises number, morphology, and function of the organelle.

In any of the embodiments herein, the totipotent or pluripotent stem cell or early extraembryonic cell can be an early extraembryonic cell. In some embodiments, the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell. In some embodiments, the one or more characteristics of differentiation comprises presence or absence of a biomarker associated with differentiation of the early extraembryonic cell.

In some embodiments, the biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. and stem cell exhaustion.

In some embodiments, the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, SIRT family members and combinations thereof.

In any of the embodiments herein, the one or more characteristics of differentiation can comprise molecular indicators of nucleotide excision repair (NER), base-excision repair (BER), DNA mismatch repair (MMR), Fanconi anemia pathway, homologous recombination (HR), nonhomologous end joining (NHEJ), variant histones, insulin resistance, pre-inflammation factors, the mTOR/AMPK pathway, mitophagy, senescence-associated secretory phenotype, and/or senescence.

In any of the embodiments herein, the totipotent or pluripotent stem cell or early extraembryonic cell can be a totipotent or pluripotent stem cell. In some embodiments, the totipotent or pluripotent stem cell is a totipotent stem cell. In some embodiments, the totipotent or pluripotent stem cell is a pluripotent stem cell.

In any of the embodiments herein, the one or more characteristics of differentiation can comprise presence or absence of a biomarker associated with differentiation of the totipotent or pluripotent stem cell.

In some embodiments, the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9mc3, H3K4mc3, H3K27mc3, H3K9ac3, SETDB1, KAP1, LaminA, LaminB1, LaminC, HP1γ, HP1α, LTR5, pTBK1, LINE1, HERVK, SIRT family members and combinations thereof.

In any of the embodiments herein, the biomarker can be a RNA molecule. In some embodiments, assessing change of one or more characteristics of differentiation comprises RNA sequencing, RT-qPCR, and/or in situ hybridization.

In any of the embodiments herein, the biomarker can be a protein molecule. In some embodiments, assessing change of one or more characteristics of differentiation comprises western blot, ELISA, proteomics, and/or immunofluorescence.

In any of the embodiments herein, the totipotent or pluripotent stem cell or early extraembryonic cell can be contacted with the candidate agent prior to being subjected to a condition for differentiation. In any of the embodiments herein, the totipotent or pluripotent stem cell or early extraembryonic cell can be contacted with the candidate agent simultaneously with being subjected to a condition for differentiation. In any of the embodiments herein, the totipotent or pluripotent stem cell or early extraembryonic cell can be contacted with the candidate agent after being subjected to a condition for differentiation.

In any of the embodiments herein, the condition for differentiation can comprise a cell culture medium comprising DMEM/F12, β-mercaptoethanol, Penicillin-Streptomycin-Glutamine, BSA, ITS-X, Y27632, Forskolin, and KnockOut Serum Replacement.

In any of the embodiments herein, the characteristics of differentiation can be evaluated at least one day after subjecting the totipotent or pluripotent stem cell or early extraembryonic cell to a condition for differentiation.

In some embodiments, the characteristics of differentiation are evaluated 2 to 8 days after subjecting the totipotent or pluripotent stem cell or early extraembryonic cell to a condition for differentiation.

In any of the embodiments herein, the totipotent or pluripotent stem cell or early extraembryonic cell can be derived from a human, a pig, a cow, a mouse, a rat, a bat, a rabbit, a dog, a cat, and a sheep.

In any of the embodiments herein, the method can further comprise determining the effect of the candidate agent on the viability of the totipotent or pluripotent stem cell or early extraembryonic cell, or cells differentiated therefrom.

In any of the embodiments herein, the method can further comprise determining the effect of the candidate agent on the viability of the totipotent or pluripotent stem cell or early extraembryonic cell, or cells differentiated therefrom.

### BRIEF DESCRIPTION OF THE DRAWINGS

FIGS. 1A-1Q show the establishment of TSC cells from expanded potential stem cells (EPSCs) and the differentiation of STBs from TSCs. FIG. 1A shows TSCs derived from M1-EPSCs. FIG. 1B shows the morphology of the established cell lines after single-clonal M1-TSC expansion was stable. FIG. 1C shows the TSCs obtained by this method expressed TSC markers (CDX2, GATA2, GATA3, CGA, KRT7, and ERVW). FIG. 1D shows expression of GATA2 and GATA3 in TSCs by immunofluorescence. FIG. 1E shows STBs differentiated from TSCs showed the expected cell morphology. FIG. 1F shows higher expression of hCG in STBs than in TSCs. FIG. 1G shows CGA and CGB3 are expressed at a higher level in STBs than in TSCs. FIG. 1H shows higher expression of hCG in STBs than in TSCs demonstrated by ELISA.

FIG. 1I shows contrasting morphologies of TSCs and STBs. FIG. 1J shows differences in cell morphology and CGB expression between TSCs and STBs. FIG. 1K shows STB markers SDC1, ERVW1, and CSH 1 were expressed significantly higher in STBs on day 6. FIG. 1L shows higher expression of hCG in STBs than in TSCs. FIG. 1M shows that STBs were at a proliferation arrest state. FIG. 1N shows decreased cell cycle scores from the RNA sequencing data as TSCs differentiated towards STBs. FIG. 1O shows STBs had significantly lower expression of proliferation marker Ki67. FIG. 1P shows STBs had significantly lower expression of proliferation marker Ki67. FIG. 1Q shows STBs had significantly higher expression of cell cycle inhibitors p27 and p38.

FIGS. 2A-2O depict changes in various aging-related markers during trophoblast stem cell (TSC)-syncytiotrophoblast (STB) differentiation. FIG. 2A shows the changes in DNA repair (nucleotide excision repair, NER). FIG. 2B shows the changes in DNA repair (base excision repair, BER). FIG. 1C shows the changes in DNA repair (DNA mismatch repair, MMR). FIG. 2D shows the changes in DNA repair (Fanconi anemia pathway). FIG. 2E shows the changes in DNA repair (homologous recombination, HR). FIG. 2F shows the changes in DNA repair (nonhomologous end joining, NHEJ). FIG. 2G shows the changes in variant histone score. FIG. 2H shows the changes in faulty nutrient sensing (insulin resistance). FIG. 2I shows the changes in pro-inflammatory factors. FIG. 2J shows the changes in faulty nutrient sensing (AMPK signaling pathway). FIG. 2K shows the changes in faulty nutrient sensing (mitophagy). FIG. 2L shows the changes in overall senescence score. FIG. 2M shows the changes in epigenetic alternation (senescence epigenetics). FIG. 2N shows the changes in senescence inhibition. FIG. 2O shows the changes in linker histone. FIG. 2P shows the changes in faulty nutrient sensing (insulin score).

FIGS. 3A-3Y depict changes in various biomarkers during TSC-STB differentiation. FIG. 3A shows different expression levels of biomarkers in TSCs and STBs on day 2, day 4, and day 6 as measured by RNA sequencing. FIG. 3B shows different expression levels of biomarkers in TSCs and STBs on day 2, day 4, and day 6 as measured by RNA sequencing. FIG. 3C shows different expression levels of biomarkers in TSCs and STBs as measured by RT-qPCR. FIG. 3D shows images of β-galactosidase staining of TSCs and STBs on day 2, day 4, and day 6. FIG. 3E shows β-galactosidase positive cells increased as TSCs differentiated towards STBs. FIG. 3F shows higher expression of γH2AX in STBs than TSCs. FIG. 3G shows higher expression of p53 in STBs than TSCs. FIG. 3H shows higher expression of gamma H2AX in STBs than TSCs. FIG. 3I shows higher expression of p53 in STBs than TSCs. FIG. 3J shows higher mitochondria mass in STBs than TSCs. FIG. 3K shows lower expression of H3K9me3 in STBs than TSCs. FIG. 3L shows lower expression of SETDB1 in STBs than TSCs. FIG. 3M shows lower expression of SIRT1, SIRT3, and SIRT6 in STBs than TSCs. FIG. 3N shows biomarkers that have higher expression in STBs than in TSCs. FIG. 3O shows expression of HP1γ and LaminB1 decreased during TSC-STB differentiation while CGA and CGB increased. FIG. 3P shows expression pf nuclear lamin proteins, LaminA and LaminB both decreased during TSC-STB differentiation. FIG. 3Q shows immunostaining images showing HP1γ highly expressed in TSCs than in STB cells. FIG. 3R shows immunostaining images showing LaminB1 highly expressed in TSCs while hCG positively expressed in STBs. FIG. 3S shows senescence-associated secretory phenotype (SASP) such as IL-6, IL-8, IL-1alpha, IL-1beta and CCL2 had higher expression in STBs than in TSCs. FIG. 3T shows classic SASP factor IL-6 co-stained with hCG in STBs with much higher expression than in TSCs. FIG. 3U shows LTR5 and HERVK expression at different time points in TSC-STB differentiation. FIG. 3V shows changes in HERVK expression in TSC-STB differentiation. FIG. 3W shows distribution of different classes of transposable elements (TE) that are upregulated and downregulated as TSCs differentiate towards STBs. FIG. 3X shows changes in γH2AX expression in TSC-STB differentiation. FIG. 3Y shows TRF1-γH2AX colocalization increases as TSCs differentiate towards STBs.

FIGS. 4A-4I show results of tests of potential anti-aging effects of various agents. FIG. 4A shows morphology of cells treated with Rapamycin. FIG. 4B shows Rapamycin reduced the expression of hCG compared to the control (DMSO). FIG. 4C shows cells treated with Rapamycin showed significantly lower CGA and CGB3 expression. FIG. 4D shows cells treated with Rapamycin showed significantly lower CKDKN1 and IL6 expression. FIG. 4E shows morphology of cells treated with Remdesivir, GC376, Molnupiravir, Rapamycin, INK128, and STM2457. FIG. 4F shows β-hCG expression levels in cells treated with Remdesivir, GC376, Molnupiravir, Rapamycin, INK128, and STM2457. FIG. 4G morphology of cells treated with Nicotinamide mononucleotide (NMN), Acarbose, Spermdine, Fisetin, and Quercetin at either 1 μM or 10 μM. FIG. 4H shows CGA expression in cells treated with NMN, Acarbose, Spermdine, Fisctin, and Quercetin at either 1 μM or 10 μM. FIG. 4I shows β-hCG expression was reduced in cells treated with Rapamycin, INK128 and Fisetin at different concentrations.

FIGS. 5A-5H show the construction of a reporter cell line and its use in testing anti-aging agents. FIG. 5A shows the design of the PD31-CGA-H2B-GFP plasmid used for making the reporter cell line. FIG. 5B shows the design of the U6-CGAgRNA-cas9 plasmid used for making the reporter cell line. FIG. 5C shows genotyping of the transfected cells. FIG. 5D shows the transfected cells expressed GFP as they differentiated into STBs. FIG. 5E shows the transfected cells increasingly expressed GFP as they differentiated towards STBs. FIG. 5F shows the transfected cells increasingly expressed GFP as they differentiated towards STBs. FIG. 5G shows the GFP signals of cells treated with various agents. FIG. 5H shows the GFP signals of cells treated with various agents.

FIG. 6 shows a schematic representation of a workflow for using TSC-STB differentiation for agent screening.

FIG. 7 shows differentially expressed genes in TSC-STB differentiation.

### DETAILED DESCRIPTION OF THE INVENTION

Stem cells and differentiated cells therefrom have extremely important prospects in areas such as regenerative medicine, disease mechanism research, therapeutic decision making, and drug screening. Traditionally, embryonic stem cells are derived from early embryos at about 100 cells, and generally do not have the ability to develop extraembryonic tissues. In recent years, expanded potential stem cells (EPSC), naive embryonic stem cells (naive ESC), and 8-cell-like totipotent stem cells (8CLC) have been reported to be able to differentiate into various types of tissues/cells, including three embryonic germ layers and trophoblasts that is extraembryonic. Because of their ability to differentiate into the extraembryonic lineage, these cells can be induced to produce various early extraembryonic cells, such as trophoblast stem cells (TSC) and trophoblast progenitor cells. Trophoblast stem cells and trophoblast progenitor cells have also been isolated from placental tissues.

During mammalian embryonic development, the placenta goes through a developmental process as the embryo develops and matures. The placenta primarily comprises trophoblast cells, which can nourish and protect the fetus. Both types of trophoblasts, syncytiotrophoblasts and extravillous trophoblasts, are derived from precursor cells called cytotrophoblasts (CTBs). In particular, cytotrophoblast cells can undergo cell fusion to give rise to syncytiotrophoblasts (STBs), which have multiple nuclei in a single cell, and can produce large amounts of human chorionic gonadotropin (hCG).

Generation of mature syncytiotrophoblasts (STBs) from cytotrophoblasts (CTBs) is accompanied by characteristics, such as growth arrest, upregulation of β-galactosidase, upregulation of cell cycle regulatory genes such as p16, p21 and p57, accumulation of heterochromatin, activation of mTOR, and shortening of telomeres.

Recent studies of embryonic stem cells suggested that mature STB and EVT can be produced in vitro by inducing TSCs to differentiate for about 8-10 days, and TSCs can be derived from trophoblasts of pre-implantation embryos or placental tissue, or very early stem cells, such as human naïve ESCs, primed ESCs or expanded potential stem cells (EPSCs). Human TSCs derived from these very early stem cells are very similar in transcriptome and epigenetics to trophoblast precursor cells (CTBs) derived from placental tissues and are considered to be the in vitro counterpart of CTBs. Very early stem cells in animals such as pigs and cattle have also been shown to be able to differentiate into trophoblast-like cells.

The invention described herein relates, in part, to systems and methods for evaluating and assessing mammalian cellular aging. The invention described herein also relates, in part, to systems and methods for evaluating potential anti-aging functions of a candidate agent and screening for such an agent in a library of candidate agents. Furthermore, the invention described herein relates, in part, to systems and methods for identifying a candidate gene that is involved in an aging process. The systems and methods disclosed herein provides a solution for rapid evaluation and screening of agents for anti-aging effects, by leveraging the differentiation process of syncytiotrophoblasts (STBs) from early extraembryonic cells or stem cells. The systems and methods disclosed herein also allows for large-scale, high throughput screening of candidate agents and candidate genes, and can therefore be used to improve the efficiency of research of anti-aging substances and aging-related target genes.

In some embodiments, the invention described herein comprises culturing early extraembryonic cells or stem cells in a condition that allows differentiation of the early extraembryonic cells or stem cells towards STBs. In some instances, the cells become mature STBs within 6-8 days and show cellular changes that match the normal mammalian aging process, such as differential expression of certain aging marker genes. The STB differentiation process from the early extraembryonic cells or stem cells therefore mimics normal mammalian aging process. In some instances, the early extraembryonic cells or stem cells comprise a reporter molecule, which signals the differentiation of an STB and/or senescence of the cells. By monitoring the cellular changes and/or the reporter molecule, the present invention can be used to evaluate the effects of an agent on cellular aging. The present invention can also be used to screen for agents that have anti-aging effects from a library of candidate agents. Additionally, the present invention can also be used to identify target genes that are involved in aging. For instance, the cells can be modified such that the expression of a reporter molecule signals the expression of the target gene during STB differentiation.

One aspect of the invention relates to a mammalian cellular aging evaluation system comprising a totipotent or pluripotent stem cell or an early extraembryonic cell, wherein the totipotent or pluripotent stem cell or early extraembryonic cell optionally comprises a reporter molecule or a heterologous nucleic acid encoding a reporter molecule that is indicative of senescence or differentiation of the totipotent or pluripotent stem cell or early extraembryonic cell towards a trophoblast or syncytiotrophoblast (“STB”).

Another aspect of the invention relates to a method of assessing an aging process of a mammalian cell, comprising subjecting a totipotent or pluripotent stem cell or an early extraembryonic cell to a condition that allows differentiation of the totipotent or pluripotent stem cell or extraembryonic cell towards a trophoblast or STB, and determining one or more characteristics of differentiation of the totipotent or pluripotent stem cell or the early extraembryonic cell.

Another aspect of the invention relates to a method of evaluating anti-aging function of a candidate agent, comprising: 1) subjecting a totipotent or pluripotent stem cell or an early extraembryonic cell to a condition that allows differentiation of the totipotent or pluripotent stem cell or early extraembryonic cell towards a trophoblast, specifically STB; 2) contacting the totipotent or pluripotent stem cell or early extraembryonic cell with the candidate agent before, during, or after subjecting the totipotent or pluripotent stem cell or early extraembryonic cell to a condition that allows the differentiation; and 3) assessing change of one or more characteristics of differentiation of the totipotent or pluripotent stem cell or early extraembryonic cell relative to a totipotent or pluripotent stem cell or an early extraembryonic cell without contacting with the candidate agent.

Another aspect of the invention relates to a method of screening for a candidate agent having an anti-aging function, comprising: 1) evaluating the anti-aging function of a plurality of candidate agents; and 2) identifying the candidate agent having an anti-aging function based on the ability of the candidate agent to cause change of one or more characteristics of differentiation of the totipotent or pluripotent stem cell or early extraembryonic cell to a trophoblast or STB relative to a totipotent or pluripotent stem cell or early extraembryonic cell without the candidate agent.

Yet another aspect of the invention relates to a method of identifying a candidate gene involved in an aging process, comprising: 1) subjecting a plurality of totipotent or pluripotent stem cells or early extraembryonic cells to a condition that allows differentiation of the plurality of totipotent or pluripotent stem cells or early extraembryonic cells towards trophoblasts or STBs, wherein each of the plurality of totipotent or pluripotent stem cells or early extraembryonic cells comprises an alteration in a candidate gene relative to a wildtype stem cell or early extraembryonic cell, wherein at least two of the plurality of totipotent or pluripotent stem cells or early extraembryonic cells contain different alterations; 2) determining one or more characteristics of differentiation of the plurality of totipotent or pluripotent stem cells or early extraembryonic cells; and 3) identifying the candidate gene involved in an aging process based on the ability of the alteration in the candidate gene to cause change of one or more characteristics of differentiation of the plurality of totipotent or pluripotent stem cell or early extraembryonic cell relative to a totipotent or pluripotent stem cell or an early extraembryonic cell without the corresponding alternation.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

A number of terms and concepts are discussed below. They are intended to facilitate the understanding of various embodiments of the invention in conjunction with the rest of the present disclosure and the accompanying figures. These terms and concepts may be further clarified and understood based on the accepted conventions in the fields of the present invention and the description provided throughout the present disclosure and/or the accompanying figures. Some other terms can be explicitly or implicitly defined in other sections of this disclosure and in the accompanying figures and may be used and understood based on the accepted conventions in the fields of the present invention, the description provided throughout the present disclosure and/or the accompanying figures. The terms not explicitly defined can also be defined and understood based on the accepted conventions in the fields of the present invention and interpreted in the context of the present disclosure and/or the accompanying figures.

As used herein, the terms “a,” “an,” and “the” can refer to “one,” “one or more” or “at least one,” unless specifically noted otherwise.

The terms “about” or “approximately” are used herein to indicate that a value includes the inherent variation of error for the device, the method being employed to determine the value, or simply error-tolerance of a value. For example, the terms “about” or “approximately” may mean±1%, ±5%, ±10%, ±15% or ±20% variation from a predetermined value.

As used herein, the terms “isolate,” “separate” or “purify” and the related terms are not used necessarily to refer to the removal of all materials other than the components of interest from a sample. Instead, in some embodiments, the terms are used to refer to a procedure that enriches the amount of one or more components of interest relative to one or more other components present in the sample. In some embodiments, “isolation,” “separation” or “purification” may be used to remove or decrease the amount of one or more components from a sample. For example, the expression “an isolated cell” can refer to a cell that has been substantially separated or purified away from other cells of a cell culture or an organism.

The term “derived” and the related expressions referring to cells or a biological sample indicate that the cell or sample was obtained from the stated source at some point in time. For example, a cell derived from an organism can represent a primary cell obtained directly from the individual (that is, unmodified), or it can be modified, for example, by introduction of a recombinant vector, by exposure to or culturing under particular conditions, or immortalization. In some cases, a cell derived from a given source will undergo cell division and/or differentiation such that the original cell no longer exists, but the continuing cells will be understood to derive from the same source. The term “derive,” “derivation” and the related terms and expressions can also be used in this disclosure to refer to creation of a cell population, cell, or culture from a different starting or preceding cell population, cell, or culture. For example, a trophoblast stem cell (TSC) described in the present disclosure can be described as being derived from Expanded Potential Stem Cells (EPSCs).

The term “comprising” and the related terms (“comprise,” “comprises,” etc.), when used in this disclosure to describe various embodiments of the invention, are open-ended, meaning that they do not exclude additional elements and synonymous with terms “including,” “containing” or “having.” When an embodiment of the invention is described using the term “comprising,” it is intended to include the embodiments, in which the term comprising is replaced with the terms “consisting of” or “consisting essentially of” In other words, the description of the embodiments of the invention described in this disclosure using the term “comprising” and the related terms also provides the description of the related embodiments that use “consisting of” or “consisting essentially of” instead of “comprising”. The term “consisting of” excludes any elements (steps, ingredient etc.) not specified in the description. The term “consisting essentially of” is intended to exclude only those elements not specified in the description that do not materially affect the basic and novel characteristics of the embodiment.

The term “lineage,” when used in reference to cells, encompasses all of the stages of the development of a cell type, from the earliest precursor cell to a completely mature cell (a specialized cell).

In the context of cell culture, the term “dissociating” can refer to a process of isolating cells from other cells or from a surface, such as a culture plate surface. For example, cells can be dissociated from an organ or a tissue by mechanical or enzymatic methods. In another example, cells that aggregate in vitro can be dissociated from each other. In yet another example, adherent cells are dissociated from a culture plate or other surface. Dissociation can involve breaking cell interactions with extracellular matrix (ECM) and substrates (for example, culture surfaces) or breaking the ECM between cells.

“Cell potency” describes a cell's ability to differentiate into other cell types. A cell can be designated as a pluripotent cell, a multipotent cell (which can differentiate into several but not all cell types, for example, umbilical cord blood stem cells and mesenchymal stem cells) or an oligopotent cell (having the ability to differentiate into a few cell types, for example, lymphoid cells or vascular cells). Under current understanding, potency exists on a continuum. Thusly, the boundaries between the divisions of cells based on potency may be fluid and are not necessarily limiting.

The expression “induced pluripotent stem cell” (iPSC) refers to a pluripotent stem cell artificially derived from a non-pluripotent cell. For example, human iPSCs are artificially derived from a human non-pluripotent cell. iPSCs can be derived by introducing products of specific sets of pluripotency-associated genes, or “reprogramming factors,” into a given cell type and/or exposing non-pluripotent cells to particular conditions. The reprogramming factors are usually active only transiently until the cells acquire pluripotent characteristics.

An “adult stem cell,” which can also be termed “somatic stem cell,” is a stem cell found, in an organism, among differentiated cells in a tissue or organ and can differentiate to yield some or all of the specialized cell times in the tissue or organ. Somatic stem cells can be grown in culture. When differentiating into specialized cells, they typically generate intermediate cells called “precursor” or “progenitor” cells. Somatic stem cells and progenitor cells can be described as “multipotent” or “oligopotent,” depending on their degree of potency. Some examples of somatic stem cells are: hematopoietic stem cells that give rise to all the types of blood cells (red blood cells, B lymphocytes, T lymphocytes, natural killer cells, neutrophils, basophils, eosinophils, monocytes and macrophages); mesenchymal stem cells that include bone marrow stromal stem cells and skeletal stem cells and can give rise to bone cells (osteoblasts and osteocytes), cartilage cells (chondrocytes), fat cells (adipocytes), and stromal cells that support blood formation; neural stem cells that can give rise to nerve cells (neurons), astrocytes and oligodendrocytes; epithelial stem cells in the lining of the digestive tract that can give rise to absorptive cells, goblet cells, Paneth cells, and enteroendocrine cells; skin stem cells that occur in the basal layer of the epidermis (and can give rise to keratinocytes) and at the base of hair follicles (and can give rise to both the hair follicle and to the epidermis). A tissue-specific progenitor cell is a cell devoid of self-renewal potential that is committed to differentiate into cells of a specific organ or tissue. Certain somatic stem cell types can differentiate into cell types seen in organs or tissues other than those expected from the somatic stem cell's origin. This phenomenon is called “transdifferentiation.”

The terms “progenitor cell” or “precursor cell,” as used herein, refers to the cells that can typically differentiate to form one or more kinds of cells. A “precursor cell” or “progenitor cell” can be any cell in a cell differentiation pathway that is capable of differentiating into a more mature cell. Progenitor cells can be primary cells obtained from an organism, cells proliferated in culture or cells derived from stem cells.

The term “placenta” and related terms refer to a temporary vascular organ in mammals, which connects the umbilical cord of the developing fetus to the wall of maternal uterus, and mediates its metabolic exchange between the fetus and the maternal blood supply through association of placental tissues with uterine mucosa.

The term “trophoblast” and related terms refer to all the cells of the trophoblast lineage, which includes a group of the extraembryonic lineages (cytotrophoblast, syncytiotrophoblast, intermediate trophoblast), and hence does not contribute directly to the cells of the fetal body. The extraembryonic lineages consist of chorion (the combination of trophoblast plus underlying extraembryonic mesoderm), amnion, yolk sac, and allantois. In some contexts, the term “trophoblast” is also used to encompass trophectoderm.

The term “trophoblast stem cell” and related terms refer to a cell of a subpopulation of trophoblast cells with stem cell properties and the ability to differentiate into either syncytiotrophoblast cells by fusion or extravillous trophoblast cells.

The terms “cytotrophoblast,” “cytotrophoblast cell” and related terms refer to a population of mononucleated cells within placental villi with stem cell and epithelial properties localized just below the syncytiotrophoblast. These trophoblast progenitors have the capacity to create either syncytiotrophoblast by fusion or extravillous trophoblast cells that invade the endometrium and remodel the maternal spiral arteries. In some contexts, the term is used interchangeably with “trophoblast stem cell” and “early extraembryonic cell”. In some instances, early extraembryonic cell is used to refer to cytotrophoblast cells cultured in vitro.

The term “syncytiotrophoblast” and related terms refer to multi-nucleated cells (which can be also be described as multinucleated structures) covering the surface of placental villi. Syncytiotrophoblasts are created by fusion of the underlying cytotrophoblast cells and represent the fetal side of the maternal-fetal interface. A distinct, early form of syncytiotrophoblast forms by fusion of trophectodermal cells in the blastocyst and facilitates implantation of the embryo into the maternal endometrium.

“Differentiation” is the process by which a less specialized cell becomes a more specialized cell type. For example, early development of a multicellular animal is characterized by the rapid proliferation of embryonic cells, which then differentiate to produce the many specialized types of cells that make up the tissues and organs of the multicellular animal. As cells differentiate, their rate of proliferation usually decreases. Some types of differentiated cells never divide again, but many differentiated cells are able to resume proliferation as required to replace cells that have been lost as a result of injury or cell death. Some cells divide continuously throughout life to replace cells that have a high rate of turnover in adult multicellular animals. Examples of differentiated cells are fibroblasts, hepatocytes, cardiomyocytes, myoblasts, neurons, osteoclasts, and lymphocytes.

The expression “modified cells” and the related terms and expressions encompass all cells that have been or are derived from the cells that have been artificially modified, by any methods, as compared to the original or cells from which they are derived. Modified cells can be produced from primary cells, secondary cells, stem cells, cultured cells and/or other modified cells. Modifications include, but are not limited to, genetic modification or engineering, in which case modified cells can be referred to as “genetically modified” or “genetically engineered.” Genetic modification can be accomplished by various methods that result in incorporation of foreign or heterologous nucleic acids into the cells being modified. Some examples of such methods are transduction by a virus or a viral vector, or transfection of isolated nucleic acids into cells through transient pores in the cell membrane. Other modifications include exposing the source cells to biological and non-biological molecules or factors or culture conditions. Some examples of modified cells are iPSCs, genetically modified cells, including those used for gene therapies, one example being gene-edited cells, such as those modified using CRISPR/Cas9, TALENs or ZFNs.

The term “passage,” “passaging” and the related terms and expressions used in the context of cell culture refer to subculturing, which typically involves transfer of cells from a previous culture into a fresh growth medium. Passaging is performed to ensure propagation of cells in culture. Cell proliferation in culture reduced or ceases when the cells reduce the capacity of the culture vessels and/or media to support further cell growth. For example, cells in adherent cultures may occupy all the available substrate and have no room left for expansion, while cells in suspension cultures exceed the capacity of the medium to support further growth. To keep cells in a culture at an optimal density for continued growth and to stimulate further proliferation, the culture must be expanded and fresh medium supplied. To divide the culture of adherent cells, for example, a monolayer culture of cells, such as cultures of differentiating EPSCs described on the present disclosure, the cells are first dissociated, for example, by enzymatic dissociation. Enzymatic dissociation can be performed by removing the incubation medium from the plates, adding to the plates a buffer, such as PBS and an enzymatic dissociation reagent, such as Accutase, TrypLE or Trypsin (available, for example, from Thermo Fisher Scientific), incubating the cells with the buffer and dissociation reagent under appropriate conditions, and harvesting the resulting dissociated cells by centrifugation, sedimentation, filtering or other appropriate methods. The dissociated cells are transferred into similar or equivalent reaction vessels, such as flasks, with fresh media, to result in a lower cell density.

As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule (for example, molecules having a molecular weight of less than 10,000 AMU). When a presence, absence of amount of a marker can be experimentally observed or detected, such a marker or its amount can be described as “observable” or “detectable.” The presence or absence of the markers, as applied to the embodiments of the preset invention, means detectable presence or absence of the markers as detected by applicable methods for detecting such markers, and may mean certain detectable or undetectable levels of such markers. In other words, the presence may mean the presence above a certain detectable level, while the absence may mean the absence below a certain detectable level and not necessarily zero detectable level. For most markers described herein, the symbols provided are those developed and/or recognized by HUGO Gene Nomenclature Committee of European Bioinformatics Institute.

In the context of observable or detectable markers, such as markers of cell development or differentiation, “expression” refers to the production of a gene product (which can be a nucleic acid, such as RNA, or a protein) as well as the level or amount of production of a gene product. Thus, determining the expression of a specific marker refers to detecting either the relative or absolute amount of the marker (which can mean detecting expression of RNA or protein) that is expressed or simply detecting (which can mean detecting expression of RNA or protein) the presence or absence of the marker. If expression of RNA or protein corresponding to the marker is detected, the marker can be said to be “detectably expressed.” Expression of certain markers can be determined by detecting the presence or absence of the marker in cells, cell culture or cell population. Expression of certain markers can also be determined by measuring the level at which the marker is present in cells, cell culture or cell population. Quantitative, qualitative or semi-quantitative techniques can be used to measure marker expression. For example, marker expression can be detected and/or quantitated through the use of techniques detecting nucleic acids, such as PCR-based detection or RNA (for example, real-time reverse-transcriptase PCR), RNA sequencing (RNA-seq), or RNA detection by nucleic acid array-based techniques. In another example, immunochemistry can be used to detect and/or quantitate marker proteins. For example, the expression of a marker gene product can be detected by using antibodies specific for the marker gene product of interest using Western blotting, immunofluorescence, flow cytometry analysis, etc. Various techniques of marker detection can be used in in conjunction to effectively and accurately characterize and identify cell types and determine both the amount and relative proportions of such markers in a subject cell type. The expression of certain markers can be determined by measuring the level at which the marker is present in the cells of the cell culture or cell population as compared to a standardized or normalized control marker. Identification and characterization of cells, cell cultures or cell population can be based on expression of a certain marker or different expression levels and patterns of more than one marker (including the presence or absence, the high or low expression, of one or more the markers). Also, certain markers can have transient expression, when the marker exhibits higher expression during one or more stages of the processes described in this disclosure and lower expression during other stage or stages.

### II. Mammalian Cellular Aging Evaluation System

The present application in one aspect provides a mammalian cellular aging evaluation system comprising a stem cell (such as a totipotent or a pluripotent stem cell) or an early extraembryonic cell, wherein the stem cell or early extraembryonic cell optionally comprises a reporter molecule or a heterologous nucleic acid encoding a reporter molecule that is indicative of senescence or differentiation of the totipotent or pluripotent stem cell or early extraembryonic cell towards a trophoblast or syncytiotrophoblast (“STB”). In some embodiments, the totipotent or pluripotent stem cell or early extraembryonic cell is naturally occurring. In some embodiments, the totipotent or pluripotent stem cell or early extraembryonic cell is genetically modified. In some embodiments, the stem cell or early extraembryonic cell comprises a reporter molecule or a heterologous nucleic acid encoding a reporter molecule. In some embodiments, the heterologous nucleic acid encoding the reporter molecule is under the control of a promoter of an endogenous biomarker gene encoding a biomarker indicative of senescence or differentiation of the stem cell or early extraembryonic cell towards a STB. In some embodiments, the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. In some embodiments, the stem cell or early extraembryonic cell is derived from a human, a pig, a cow, a mouse, a rat, a bat, a rabbit, a dog, a cat, or a sheep.

The present application in one aspect provides a mammalian cellular aging evaluation system comprising a stem cell (such as a totipotent or a pluripotent stem cell) or an early extraembryonic cell, wherein the stem cell or early extraembryonic cell comprises a reporter molecule that is indicative of senescence or differentiation of the totipotent or pluripotent stem cell or early extraembryonic cell towards a trophoblast or a syncytiotrophoblast (“STB”). In some embodiments, the stem cell or early extraembryonic cell is derived from a human, a pig, a cow, a mouse, a rat, a bat, a rabbit, a dog, a cat, or a sheep.

The present application in one aspect provides a mammalian cellular aging evaluation system comprising a stem cell (such as a totipotent or a pluripotent stem cell) or an early extraembryonic cell, wherein the stem cell or early extraembryonic cell comprises a heterologous nucleic acid encoding a reporter molecule that is indicative of senescence or differentiation of the totipotent or pluripotent stem cell or early extraembryonic cell towards a trophoblast or syncytiotrophoblast (“STB”). In some embodiments, the heterologous nucleic acid encoding the reporter molecule is under the control of a promoter of an endogenous biomarker gene encoding a biomarker indicative of senescence or differentiation of the stem cell or early extraembryonic cell towards a trophoblast or STB. In some embodiments, the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. In some embodiments, the stem cell or early extraembryonic cell is derived from a human, a pig, a cow, a mouse, a rat, a bat, a rabbit, a dog, a cat, or a sheep.

The present application in one aspect provides a mammalian cellular aging evaluation system comprising a pluripotent stem cell, wherein the pluripotent stem cell optionally comprises a reporter molecule or a heterologous nucleic acid encoding a reporter molecule that is indicative of senescence or differentiation of the pluripotent stem cell towards a syncytiotrophoblast (“STB”). In some embodiments, the pluripotent stem cell comprises a reporter molecule or a heterologous nucleic acid encoding a reporter molecule. In some embodiments, the heterologous nucleic acid encoding the reporter molecule is under the control of a promoter of an endogenous biomarker gene encoding a biomarker indicative of differentiation of the pluripotent stem cell towards a STB. In some embodiments, the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. In some embodiments, the pluripotent stem cell is derived from a human, a pig, a cow, a mouse, a rat, a bat, a rabbit, a dog, a cat, or a sheep.

The present application in one aspect provides a mammalian cellular aging evaluation system comprising a trophoblast stem cell (“TSC”), wherein the TSC optionally comprises a reporter molecule or a heterologous nucleic acid encoding a reporter molecule that is indicative of differentiation of the TSC towards a syncytiotrophoblast (“STB”). In some embodiments, the TSC comprises a reporter molecule or a heterologous nucleic acid encoding a reporter molecule. In some embodiments, the heterologous nucleic acid encoding the reporter molecule is under the control of a promoter of an endogenous biomarker gene encoding a biomarker indicative of differentiation of the TSC towards a STB. In some embodiments, the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. In some embodiments, the TSC is derived from a human, a pig, a cow, a mouse, a rat, a bat, a rabbit, a dog, a cat, or a sheep.

### 1. Reporter Molecules and Heterologous Nucleic Acid Encoding a Reporter Molecule

In some embodiments, the stem cell or early extraembryonic cell comprises a reporter molecule or a heterologous nucleic acid encoding a reporter molecule that is indicative of differentiation of the engineered extraembryonic cell towards a syncytiotrophoblast (“STB”).

In some embodiments, the stem cell or early extraembryonic cell comprises a reporter molecule. Examples of reporter molecules include physically activated molecules and chemically activated molecules. The reporter molecule could be fluorescent or non-fluorescent. Examples of physically activated molecules include but are not limited to light activated sensing molecules, such as GFP, RFP, mCherry, photoconvertible fluorescent proteins, etc. Examples of chemically activated molecules include but are not limited to: 1) enzyme-activated sensing molecules, such as bioluminescence (e.g., luciferase) and enzyme assays (e.g., β-galactosidase, β-glucuronidase, and β-lactamase); 2) antibody-based assays, such as IF antibody assays; 3) chloramphenicol acetyltransferase; and 4) biosensors (e.g., probes).

In some embodiments, the stem cell or early extraembryonic cell comprises a heterologous nucleic acid encoding a reporter molecule. The heterologous nucleic acid may be integrated into the genome of the stem cell or early extraembryonic cell by techniques such as gene editing as described in Section II.2. In some embodiments, the heterologous nucleic acid is under the control of a promoter of an endogenous biomarker gene encoding a biomarker indicative of senescence or differentiation of the stem cell or early extraembryonic cell towards a trophoblast or STB. As a result, if the promoter is being actively expressed within the cell, the reporter gene will also be expressed, which can be detected/measured. Reporter genes can produce a protein that has little obvious or immediate effect on the cell culture or organism. They are ideally not present in the native genome to be able to isolate reporter gene expression as a result of the gene of interest's expression. Reporter genes can be incorporated genetically into the host DNA of individual cells.

Reporter genes replace the stop codon of the gene of interest to create a gene fusion, so that they can be expressed with the gene of interest. Also, in building the reporter gene system, a segment of DNA coding for a flexible polypeptide linker region such as T2A and IRES is usually inserted right in front of the reporter genes. This method is an example of using cis-acting elements where the two genes are under the same promoter elements and are transcribed into a single messenger RNA molecule. The mRNA is then translated into protein, and linker region like T2A or IRES mediates co-translational cleavage In this way, both proteins be able to properly fold into their active conformations instead of becoming a fusion protein. The reporter and the product of the gene of interest will only minimally interfere with one another.

Reporter genes can also be under the control of a transcriptional regulatory complex (e.g. a promoter) that is inducible, with the transcriptional regulatory elements responding to endogenous cell signals (e.g., transcription factors and transcription-regulating complexes) or exogenous chemical or physical conditions that can initiate and regulate reporter gene expression.

A reporter system typically includes 2 components: a specific gene and regulatory complex, and a specific substrate that interacts with the gene product. The reporter gene product is a protein-either an enzyme that catalyzes a chemical reaction or a protein that fluoresces on exposure to light. Examples of commonly used reporter system pairs include radionuclide-based pairs (e.g., HSV1-tk [herpes simplex virus type 1 thymidine kinase] and 124/131I-FIAU [5-iodo-2′-fluoro-2′deoxy-1-β-d-arabinofuranosyluracil] or 18F-FEAU [2′-deoxy-2′-18F-fluoro-5-ethyl-1-β-d-arabinofuranosyluracil]), bioluminescent pairs (e.g., firefly luciferase [FLuc] and d-luciferin), and fluorescent pairs (e.g., green fluorescent protein [GFP] and activating blue light), plus sensors exploiting fluorescence resonance energy transfer between 2 mutant GFP molecules.

In some embodiments, the reporter molecule is selected from the group consisting of Green Fluorescent Protein (GFP), Red Fluorescent Protein (RFP), Yellow Fluorescent Protein (YFP), mCherry, tdTomato and photoconvertible fluorescent proteins. When expressed, the reporter molecule can be detected using methods such as fluorescent microscopy and flow cytometry (See, e.g., Kremers et al., J Cell Sci. 2011, 124 (2): 157-160; Chudakov et al., Physiol Rev. 2010, 90 (3): 1103-63.)

In some embodiments of the system disclosed herein, the stem cell or early extraembryonic cell may comprise more than one reporter molecule or more than one heterologous nucleic acid encoding a reporter molecule, or a combination thereof. For example, an early extraembryonic cell used in the system disclosed herein may comprise a first heterologous nucleic acid encoding a Green Fluorescent Protein under the control of a promoter of gene A and a second heterologous nucleic acid encoding a Red Fluorescent Protein under the control of a promoter of gene B. The differentiation of the cell can be assessed by the intensity of the Green Fluorescent Protein and the Red Fluorescent Protein, indicating the expression level of gene A and gene B, respectively.

### 2. Cells for Evaluating Mammalian Cellular Aging

One embodiment of the present disclosure is a mammalian cellular aging evaluation system comprising a stem cell (such as a totipotent or a pluripotent stem cell) or an early extraembryonic cell. In some embodiments, the stem cell or early extraembryonic cell is naturally occurring.

The mammalian cellular aging evaluation system may comprise a stem cell that is capable of producing cells of the trophoblast lineage. The mammalian cellular aging evaluation system may comprise a totipotent stem cell or a pluripotent stem cell. Totipotent stem cells are cells that have the capacity to self-renew by dividing and to develop into the three primary germ cell layers of the early embryo and into extraembryonic tissues such as the placenta. Totipotency exists transiently in zygote and 2-cell embryo stages during early development, which subsequently commit to two distinct lineages, i.e., the embryonic cell lineage (inner cell mass, ICM) that forms embryo proper and the extraembryonic cell lineage (trophectoderm, TE) that forms the placental tissue. Pluripotent stem cells including embryonic stem cells (ESCs) and induced pluripotent stem cells (iPSCs) have the potential to differentiate into all somatic cell types. Recent studies have shown that pluripotent stem cells could be programmed to produce extraembryonic cells such as trophoblast stem cells (Dong et al., 2020, eLife 9:e52504).

Methods for obtaining totipotent stem cells and induced pluripotent stem cells are known in the art. Totipotent stem cells could be obtained by methods such as transferring a somatic nucleus into an enucleated oocyte, which is also known as the somatic cell nuclear transfer (SCNT) technique. The SCNT procedure involves three major steps: enucleation, injection/fusion, and activation. After removing the oocyte nucleus, the donor cell nucleus is injected or fused with the enucleated oocytes before the reconstructed embryos are activated. Successful cloning of more than 20 mammalian species using SCNT has been reported (Matoba et al., Cell Stem Cell. 2018 Oct. 4; 23 (4): 471-485). In addition to animal cloning, SCNT technology holds great potential for stem cell biology and human therapeutics. Similar to the derivation of embryonic stem cells (ESCs) from blastocysts of fertilized eggs, SCNT-generated blastocysts could be used to derive pluripotent stem cells.

Induced pluripotent stem cells (iPSCs) are typically derived by introducing a specific set of pluripotency-associated genes, or “reprogramming factors,” into an adult cell type. The original set of reprogramming factors (also called Yamanaka factors) are the genes Oct4 (Pou5f1), Sox2, cMyc, and Klf4. There are multiple methods to generate iPSCs, including retrovirus or lentivirus-mediated gene transduction and chemical induction. To generate the iPSCs, each of the pluripotency factors can be also replaced by related transcription factors, miRNAs or small molecules (Ghaedi et al., Methods Mol Biol. 2019; 1576:55-92).

In some embodiments, the mammalian cellular aging evaluation system comprises an early extraembryonic cell. In some embodiments, the early extraembryonic cell is a trophoblast stem cell (“TSC”) or a trophoblast progenitor cell (“TPC”). In some embodiments, the early extraembryonic cell is a TSC. In some embodiments, the early extraembryonic cell is derived from a totipotent stem cell, a pluripotent stem cell, an embryo tissue, or a placenta tissue. In some embodiments, the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

In one particular embodiment, the TSC is derived from an expanded potential stem cell (EPSC). EPSCs derived from cleavage-stage preimplantation embryos retain developmental potential for both extraembryonic and embryonic cell lineages (Yang et al., 2017, Cell 169, 243-257.e25; Yang et al., 2017, Nature 550, 393-397; Ruan et al., 2022, Cell Reports Medicine 3, 100849; Gao et al., 2019, Nat. Cell Biol. 21, 687-699). Methods for inducing EPSCs to differentiate into TSCs are known in the art. An exemplary protocol is described in Okae et al., 2018, Cell stem cell, 22 (1), 50-63, the content of which is incorporated herein in its entirety. In some embodiments, methods for inducing EPSCs to differentiate into TSCs comprises culturing the cells in a cell culture medium comprising DMEM/F12, β-mercaptoethanol, FBS, Penicillin-Streptomycin, bovine albumin fraction V (BSA), Insulin-Transferrin-Selenium-Ethanolamine (ITS-X) supplement, 2-phospho-L-ascorbic-acid (Vc), EGF, CHIR99021, A83-01, SB431542, Valproic acid (VPA), and Y27632. In some embodiments, the cell culture medium comprises DMEM/F12 supplemented with 110 μM β-mercaptoethanol, 0.2% FBS, 0.5% Penicillin-Streptomycin, 0.3% BSA, 1×ITS-X supplement, 50.0 μg/mL Vc, 50.0 ng/mL EGF, 2.0 μM CHIR99021, 0.5 μM A83-01, 1.0 μM SB431542, 0.8 μM VPA, and 5.0 μM Y27632.

In some embodiments, the TSC is derived from a naive pluripotent stem cell (e.g., a naïve embryonic stem cell). Naive pluripotent stem cells differ from primed pluripotent stem cells in that primed pluripotent stem cells are poised for lineage commitment. One type of naive pluripotent stem cells, naïve embryonic stem cells (ESCs) readily differentiate to somatic or germ lineages but have impaired ability to form extra-embryonic lineages such as placenta or yolk sac. Recent studies have shown that human naïve ESCs can be transdifferentiated to cells that exhibit the cellular and molecular phenotypes of human trophoblast stem cells (hTSCs) derived from human placenta or blastocyst. An exemplary protocol is described in Cinkornpumin et al., 2020, Stem Cell Rep, 15, 198-213, the content of which is incorporated herein in its entirety.

In some embodiments, the stem cell or early extraembryonic cell is genetically modified. For instance, in some embodiments, the stem cell or early extraembryonic cell comprises a heterologous nucleic acid and the heterologous nucleic acid is introduced into the stem cell or early extraembryonic cell by a gene editing tool. Examples of gene editing tools include but are not limited to (1) clustered regularly interspaced short palindromic repeats (CRISPR)-CRISPR-associated protein (Cas), (2) transcription activator-like effector nucleases (TALENs), (3) zinc-finger nucleases (ZFNs), and (4) homing endonucleases or meganucleases.

### 3. Endogenous Biomarkers

Many endogenous biomarkers may be suitable for the system disclosed herein. In some embodiments, the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, senescence-associated secretory phenotype, and stem cell exhaustion.

In some embodiments, the endogenous biomarker is selected from the group consisting of PEG3, DDX3Y, RPS4Y1, TXLNGY, KDM5D, USP9Y, THEM6, CAT, ZFY, LYNX1, EIF1AY, PLAC4, LAIR2, WIPF1, APOC3, LY6K, ZNF558, OAS3, LINC02055, LGALS14, ZNF826P, TTTY14, NLGN4Y, TMSB4Y, SEC14L4, LINC01446, ZFP41, LGALS13, ZNF257, STEAP4, GH2, MAG, LINC00278, TPTEP1, CD22, AIRE, CNN1, APOA5, ZFY-AS1, OAS2, ANOS2P, GNG4, CMKLR2, OXGR1, HOXB7, RLN2, WNT9B, LINC01949, COX20P1, SPANXN5, NBPF6, PRKY, IGHV3-74, CSH2, UNC13A, BACE2, TMC1, LINC01090, CYP26A1, PSCA, PAEP, UTY, MIR4482, ZNF835, RN7SL708P, ZNF717, WFDC21P, FLT4, NOC2LP1, NBPF4, LY6D, ZNF736P9Y, OR2C3, FAM167A, GABRE, GYG2P1, ZNF630, HLF, KCNS3, MTNR1B, NPR, HOXC9, LINC02701, ZNF726, WSCD1, KRT78, ETV7, ZNF658, GPC4, NKAIN4, FLJ16779, RERG, BGN, DLX5, HTRA4, SCUBE1, LINC00323, LY6E, NCF1C, GJA3.

In some embodiments, the endogenous biomarker is selected from the group consisting of DDX3Y, PEG3, RPS4Y1, PLAC4, TXLNGY, KDM5D, CSH2, LGALS14, OAS3, OAS2, ZFY, GH2, WIPF1, HLF, LAIR2, USP9Y, STEAP4, THEM6, CAT, LYNX1, LY6K, APOC3, EIF1AY, ADAM12, CSH1, ZNF558, IFNL3, LGALS13, SCUBE1, SPDYE13, WNT9B, FLT4, LINC02055, CD22, TTTY14, ZFP41, IFIT2, MMEL1, XAF1, TMSB4Y, CMKLR2, LINC01446, KCNS3, GPC3, IFNL2, LAMA4, MAG, LINC00278, ABCB1, LINC01949, MUCL1, TPTEP1, SPANXN5, KCNJ2, HTRA4, ZNF826P, APOA5, AIRE, OASL, SPTA1, NLGN4Y, PDK4, PTPN22, GUCY2F, PAEP, PSCA, HTRA1, OXGR1, ACVRL1, MAN1C1, ARNT2, H2BC8, SEC14L4, BGN, UNC5A, MIR100HG, NBPF6, IFIT3, GBP1, ZFY-AS1, NPR 1.00, RLN2, BACE2, ZNF257, CGB2, IGHV3-74, NBPF4, LINC02701, IFNL1, GNG4, ZNF835, CNN1, FAM167A, GPR143P, KRT78, RN7SL708P, CGB1, LY6D, LINC02533, TMEM225B.

In some embodiments, the endogenous biomarker is selected from the group consisting of OAS2, PEG3, DDX3Y, OAS3, CSH2, RPS4Y1, GH2, CSH1, LAIR2, PLAC4, LGALS14, KDM5D, TXLNGY, THEM6, WIPF1, LYNX1, XAF1, IFNL3, ZFY, LAMA4, APOC3, ADAM12, USP9Y, STEAP4, HLF, IFNL2, SPDYE13, ZNF558, CAT, LY6K, EIF1AY, CD22, IFIT2, IFIT3, OAS1, SCUBE1, CSHL1, GPC3, LINC02055, WNT9B, CNN1, FLT4, BST2, SAMD9L, ZFP41, GBP1, CMKLR2, MUCL1, MX1, IFNL1, OASL, LINC01446, SPANXN5, KCNJ2, ETV7, NLGN4Y, KCNS3, IFITM1, MAG, TPTEP1, TTTY14, LGALS13, MX2, KCNK3, SEMA3B, PDK4, HTRA1, APOA5, FLJ16779, IGHA1, TAC3, ZNF826P, GNG4, HTRA4, CYP26A1, SHFL, GPC4, TCAM1P, WFDC21P, ITIH3, KRT78, SPDYE9, EPHB2, ANGPTL4, SPDYE11, PSAPL1, PSCA, IFIT1, IFI27, MYO1A, PAEP, LINC01949, LINC02533, FGF21, ESAM, SLC16A2, FN1, SAMD9, NBPF6, GTF2I-AS1.

In some embodiments, the endogenous biomarker is selected from the group consisting of OAS2, OAS3, PEG3, DDX3Y, LGALS14, RPS4Y1, PLAC4, CSH2, STEAP4, IFNL3, GH2, IFNL2, CSH1, KDM5D, HLF, USP9Y, LYNX1, LAIR2, TXLNGY, THEM6, ZFY, XAF1, IFIT2, ADAM12, WIPF1, APOC3, CAT, CNN1, ZNF558, EIF1AY, PDK4, IFIT3, IFNL1, MYO1A, SAMD9L, LY6K, SPANXN5, GPC3, CSHL1, OASL, OAS1, SPDYE13, FLJ16779, TAC3, PAPPA, ZFP41, WNT9B, NLGN4Y, IGHA1, GBP1, TRIM40, SPTA1, MX1, LINC01949, FLT4, KCNJ2, LINC02055, TTTY14, LINC02533, TPTEP1, NKAIN4, CMKLR2, KRT17P2, CCL22, LINC01446, ETV7, SEMA3B, LAMA4, MT-TH, MMEL1, BST2, LGALS13, WFDC21P, GUCY2F, MUCL1, CXCL10, FAM167A, ACVRL1, LINC00278, IFI27, SAMD9, IFITM1, SCEL, ZNF826P, CD22, AIRE, NCF1C, FN1, MAN1C1, CYP26A1, SHFL, DLGAP1, SLC16A2, KRT78, PSCA, UNC13A, APOA5, ANGPTL4, NBPF6, CPE.

In some embodiments, the endogenous biomarker is selected from the group consisting of MAGEA2B, MAGEA2, MAGEA12, CSAG1, MAGEB2, SH3BGRL, CSAG3, LINC02413, TSSC2, PTCHD1, HMGA2, ARHGEF9, GALNT14, FAM86GP, CSAG2, AJAP1, LPAR1, TLR4, DDX11L2, OR7E12P, CDCA7L, LINC01405, PI15, LINC02474, DCLK1, SNHG14, NDN, PRKCQ, PINCR, CDX1, IMPDH1P4, RPE65, UCP1, MSN, CHMP1B2P, SNTB1, BMP5, DPYSL4, FBXW12, STAMBPL1, A2M, KCNJ5, E2F6P4, ALOX12P2, NETO1, LRRTM4, KRT72, ATP8A1, PRKCQ-AS1, PCDHB2, CSAG4, AMER1, LINC01291, LINC01807, LINC02412, MIR2052HG, NCAM1, PCDHA4, MYEOV, CDH13, ALCAM, ARTN, PCDHA10, TRIM61, TEX41, PCDHA12, PTPRB, CYP2C8, PROM1, PCDHA11, FAM110C, TGFA, KRTAP2-3, ZNF57, NACAP8, IGSF1, MPV17L, SIMC1, BUD13P1, NRROS, UGT1A6, STK32B, ROR2, HMGN5, LINC02617, LINC00379, WDR90, PLA2G4A, CASC9, CYP4F26P, TEX35, LINC02154, GSTT2B, IFFO1, CELF2, CHRM3, C1RL-AS1, RARRES1, RGPD2, HOMER2.

In some embodiments, the endogenous biomarker is selected from the group consisting of MAGEA12, CSAG1, MAGEA2B, MAGEA2, MAGEB2, SH3BGRL, LINC02413, TSSC2, CDX1, ARHGEF9, CHRM3, GALNT14, AJAP1, CSAG3, ALCAM, TLR4, TGFA, DDX11L2, OR7E12P, PTCHD1, CDCA7L, HMGA2, PI15, MYEOV, LINC02474, PRKCQ-AS1, LINC02617, PINCR, HMGN5, FAM162B, NDN, EYA1, PRKCQ, LINC01405, PCDHA10, PCDHA12, RPE65, ZNF57, CLDN10, SFTA1P, CHMP1B2P, SNTB1, C10orf82, DPYSL4, MSN, FBXW12, STAMBPL1, E2F6P4, FAM83A, LINC01127, NETO1, CSAG2, KRT72, ATP8A1, SNX18P7, CSAG4, COL19A1, LINC01807, LINC02458, LINC02412, MIR2052HG, NCAM1, LPAR1, NECTIN1-DT, SLC7A11-AS1, KCTD15, LINC00379, ITGB1BP2, PROM1, UGT1A1, C1orf220, CA8, CDH13, ZSCAN16-AS1, RARRES1, TRIM61, ANKRD18B, EML5, PRKG2, PRPH2, FHL1, FAM86GP, RBP5, UGT1A6, BMP5, STK32B, CLEC20A, TEX35, GLUD2, CERS6, SNHG14, KLHL4, C1RL, PTPRB, LINC00668, CHST6, ITPRIPL1, GSTT2B, TERT, DCLK1.

In some embodiments, the endogenous biomarker is selected from the group consisting of MAGEA2B, MAGEA2, MAGEA12, TSSC2, CSAG1, MAGEB2, CSAG3, LINC02413, HMGA2, ARHGEF9, FAM86GP, CSAG2, AJAP1, GALNT14, OR7E12P, PTCHD1, LINC02474, DCLK1, PRKCQ-AS1, PINCR, SNHG14, FAM162B, PRKCQ, CYP4F26P, MSN, PCDHA10, EML5, LINC00379, PCDHA12, IMPDH1P4, CDX1, RPE65, ZNF57, CHMP1B2P, TLR4, PK1A, DSG2-AS1, UGT1A6, DDX11L2, LINC01405, BMP5, C10orf82, DPYSL4, STAMBPL1, WHAMMP2, HAPLN1, PTPRB, PLA2G4A, NETO1, KRT72, ST13P20, PCDHB2, SNX18P7, HMGN5, MAP10, CSAG4, LINC02617, MIR3176, UCP1, LINC01807, NDN, LINC02412, MIR2052HG, NCAM1, CYP1A1, SH3BGRL, CELF2, ARMC3, UGT1A1, ZSCAN16-AS1, NR0B1, TRIM61, DIAPH2-AS1, SAPCD1, CYP2C8, PCDHA11, MRPS18AP1, PCDHB5, KRTAP2-3, NACAP8, CDKL2, FAM83A, DOC2GP, LPAR1, SNTB1, RARRES1, RBP5, LINC02575, DEPDC1, MECOM, KCNE3, ALCAM, ABHD12B, SSBP3-AS1, GLUD2, A2M, CHST6, HPDL, AK4, FGFBP1.

In some embodiments, the endogenous biomarker is selected from the group consisting of MAGEA2B, MAGEA2, MAGEA12, GALNT14, CSAG1, MAGEB2, LINC01405, CDX1, PTCHD1, ARHGEF9, AJAP1, LINC02413, TLR4, DDX11L2, OR7E12P, LINC02474, DCLK1, LINC02575, PRKCQ-AS1, CERS6, CHRM3, NDN, PRKCQ, CYP4F26P, TSSC2, PCDHA10, PIMREG, IMPDH1P4, LINC00379, RPE65, ZNF57, SH3BGRL, PINCR, SNTB1, UGT1A6, BMP5, C10orf82, DPYSL4, STAMBPL1, E2F6P4, PI15, PNPO, NETO1, LRRTM4, KRT72, ATP8A1, PCDHB2, SNX18P7, HMGN5, CSAG4, COL19A1, PROM1, LINC01807, LINC02412, MIR2052HG, NCAM1, NECTIN1-DT, TEX35, DMC1, LINC02617, FAM86GP, TERT, TRIM61, EML5, TEX41, PCDHA12, PCDHA11, CSAG3, TSPEAR-AS2, RARRES1, LINC00668, ZNF785, MPV17L, EVX1, FAM83A, CHMP1B2P, ABHD12B, PK1A, C1RL-AS1, STK32B, LINC02582, CTTNBP2, TGFA, CTAGE3P, SNORD99, AMER1, LINC01291, ITPRIPL1, FAM162B, MCM10, AK4, MSN, KRT3, CHST6, HHLA3-AS1, CSAG2, NR0B1, EMB, KCNE3, UCP1.

In some embodiments, the endogenous biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, and SIRT family members.

In some embodiments of the present invention, the endogenous biomarker used is a gene that increases expression during normal mammalian cellular aging. In some embodiments, the endogenous biomarker used also shows increasing expression in the mammalian cellular aging evaluation system. For example, the expression of the endogenous biomarker increases as the stem cell or early extraembryonic cell ages or differentiates towards a STB. As the expression of the endogenous biomarker increases, the expression of the reporter molecule also increases. The increase in expression can be detected as described above, therefore signaling the senescence or differentiation of STB.

In some embodiments, the endogenous biomarker used is a gene that decreases expression during normal mammalian cellular aging and in the mammalian cellular aging evaluation system. For example, the expression of the endogenous biomarker decreases as the stem cell or early extraembryonic cell differentiates towards a STB. As the expression of the endogenous biomarker decreases, the expression of the reporter molecule also decreases. Examples of such suitable endogenous biomarkers include HP1gamma, Lamins and SIRT family members.

In some embodiments, the endogenous biomarker is a marker of genomic instability. In some embodiments, the endogenous biomarker is HP1gamma, HP1 alpha, LaminA/C, LaminB1, or SETDB1/KAP1 complex.

In some embodiments, the endogenous biomarker is a marker of DNA damage. Suitable markers include but are not limited to γH2AX and P53. H2AX is a histone mammalian variant that belongs to the H2A family. Histones are proteins that construct the nucleosomes, the basic unit of chromatin. When double-strand breaks are generated into DNA, H2AX becomes rapidly phosphorylated at serine 139. This specific phosphorylation is denoted as “γ-phosphorylation”, and the term “γH2AX” indicates the specific phosphorylation at serine 139 of the histone H2AX. γH2AX is an early sign of DNA damage induced by replication stalling. p53 plays a prominent role as a facilitator of DNA repair by halting the cell cycle to allow time for the repair machineries to restore genome stability. It has also been directly implicated in DNA-repair pathways (e.g., remodeling chromatin during nucleotide excision repair).

Additionally, the biomarker could be a marker for DNA repair. DNA damage can be caused by a multitude of factors, but ultimately takes the form of either chemical modification to a DNA base, the presence of single-stranded DNA (ssDNA) or ssDNA break or of a more severe DNA-DSB. The repair of lesions generated by chemical modifications to DNA bases, such as oxidative lesions or bulky adducts, are rapidly processed by the nucleotide excision repair (NER) and base excision repair (BER) pathways. DNA can be damaged when cells are exposed to mutagens, which could distort the double helix structure or prevent correct transcription. Damaged DNA can be detected and repaired by the process of nucleotide excision repair (NER). Studies have found that all cells have different types of glycosylase that can recognize damaged nucleotides. Glycosylases can specifically cut N-β-glycosidic bonds on damaged bases, generating abasic (AP) sites. Once AP sites are generated in the DNA molecule, an AP-endonuclease cleaves the AP site, generating 3′ OH and 5′ deoxyribose phosphate (dRP) terminus, and removing a small fragment of DNA including the AP site. The DNA Polymerase I then synthesizes new fragments, and finally DNA ligase connects the two into a new repaired DNA strand. This process is called base-excision repair (BER). It represents the DNA repair mechanism for damage. The markers relevant to these cellular processes are shown in Table 2.

The biomarker can also be selected from other pathways for DNA repair including 1) DNA mismatch repair (MMR), which can correct mismatches that occur occasionally during DNA replication and recombination, and the mismatched bases can be recognized and repaired by mismatch repair enzymes; 2) the Fanconi anemia signaling pathway which is considered necessary for effective repair of damaged DNA; 3) homologous recombination (HR) which provides high-fidelity, template-dependent repair or tolerance of complex DNA damages including DNA gaps, DNA double-stranded breaks, and DNA interstrand crosslinks; and 4) nonhomologous end joining (NHEJ) which repairs double-strand breaks in DNA by directly ligating DNA without the need for a homologous template.

TABLE 2
DNA Damage Markers

| DNA | |
|---|---|
| damage | |
| marker | Damage type/Repair mechanism |
| ATM | DSB |
| ATR | SSB |
| ATRIP | SSB |
| CDC25A/B/C | SSB, DSB |
| CHK1 | SSB |
| CHK2 | DSB |
| DDB1 | SSB, NER |
| DDB2 | SSB, NER |
| H2AX | DSB |
| KU70, KU80 | DSB, NHEJ |
| MRE11 | DSB, HR |
| NBS1 | DSB, HR |
| PCNA | SSB, NER |
| POLD1/2/3/4 | SSB |
| POLE3 | SSB |
| POLK | SSB |
| RAD1 | SSB |
| RAD51 | DSB, HR |
| FEN1 | DSB, MMR |
| RFC2/3/4/5 | SSB, NER |
| RPA1/2/3/4 | SSB, DSB, HR |

In some embodiments, the endogenous biomarker is a marker of epigenetic alternations. During the process of aging, extensive epigenetic alterations are made in response to both exogenous and endogenous stimuli. Examples of epigenetic-related markers include but are not limited to H3K9me3, H3K4me3, H3K9ac3, and linker histone H1. In some embodiments, the endogenous biomarker is H3K9me3.

In some embodiments, the endogenous biomarker is a differentiation marker. Examples of differentiation markers include but are not limited to CDX2, KRT7, HLA-G, ID2, CGA, CGB, ERVW family members, CSH1, SDC1, and PSG1. CGA and CGB are involved in hormonal processes and have been shown to increase in expression during STB differentiation (Marchand et al., Biology of Reproduction, Volume 84, Issue 6, 1 Jun. 2011, Pages 1258-1271; Ruan et al., 2022, Cell Reports Medicine 3, 100849). The differentiation of TSC towards STB is primarily driven by human endogenous retroviruses (HERV), specifically Syncytin-1 (a product of the ERVW-1 gene) and Syncytin-2 (ERVFRD-1), which are critical drivers of CTB fusion. CSH1 (Chorionic somatomammotropin hormone 1), SDC1 (Syndecan1), and PSG1 (pregnancy specific beta-1-glycoprotein 1) are highly enriched in STBs. The transcription factor CDX2 acts early during the blastocyst formation, playing an instructive role in the formation of trophoblast. KRT7, HLA-G, and ID2 are also markers for STB differentiation. Other suitable differentiation markers include DLX3, GATA3, DAB2, TEAD3, and TFAP2C. In some embodiments, the endogenous biomarker is CGA.

In some embodiments, the endogenous biomarker is a endogenous transposon element. Examples of endogenous transposon elements markers include but are not limited to HERVK (human endogenous retrovirus K), pTBK1, and LINE1. In some embodiments, the endogenous biomarker is HERVK. As reported in Liu et al., 2023, Cell 186, 287-304, upregulation of HERVK triggers young cells to enter cellular senescence. HERVK is shown to be highly expressed in STB differentiation around day 2, which belongs to an initiation part of TSC-STB ageing process.

In some embodiments, the endogenous biomarker is a cell cycle-related marker. Examples of cell cycle-related markers include but are not limited to p27 and p38. P27 binds and inhibits cyclin-CDK to arrest the cell cycle, and was shown to be expressed in the differentiated, non-dividing STBs. P38 is essential to mediate initiation of STB differentiation, and is expressed highly early in TSC-STB differentiation.

In some embodiments, the endogenous biomarker is a marker of telomere attrition. Examples of cell cycle-related markers include but are not limited to TRF1. TRF1 encodes a telomere specific protein which is a component of the telomere nucleoprotein complex. This protein is present at telomeres throughout the cell cycle and functions as an inhibitor of telomerase, acting in cis to limit the elongation of individual chromosome ends.

In some embodiments, the endogenous biomarker is a marker of cellular senescence. Cellular senescence is a process that globally regulates cell fate and can be considered a hallmark of aging. It is associated with multiple cellular, molecular changes and distinct phenotypic alterations including a stable and generally irreversible proliferation arrest unresponsive to mitogenic stimuli. One characteristic feature of senescent cells is increased lysosomal activity, macromolecular damage, and a temporal cascade in the development of the complex senescence-associated secretory phenotype (SASP). Examples of markers of senescence include but are not limited to CDKN1A, CDKN2A, CDKN2B, BCL2 family members, NF-κB, FOXO3, SMAD3, which play important roles in regulating senescence. For example, CDKN1A encodes p21^{WAF1/CIP1}, a protein that is capable of inactivating all CDKs, thereby inhibiting cell cycle progression. NF-κB is generally recognized as an important regulator of ageing, through its roles in cellular senescence and inflammatory pathways. In some embodiments, the marker of senescence is senescence-associated β-galactosidase (SA-β-gal) activity,

In some embodiments, the endogenous biomarker is a marker of senescence-associated secretory phenotype (SASP). SASP is a phenotype associated with senescent cells wherein those cells secrete high levels of inflammatory cytokines, immune modulators, growth factors, and proteases. SASP may also consist of exosomes and ectosomes containing enzymes, microRNA, DNA fragments, chemokines, and other bioactive factors. SASP is one of the three main features of senescent cells, the other two features being arrested cell growth, and resistance to apoptosis. SASP expression is induced by a number of transcription factors, including C/EBPβ, of which the most important is NF-κB. SASP factors cause non-senescent cells to become senescent, induce insulin resistance, and disrupt normal tissue function by producing chronic inflammation, induction of fibrosis and inhibition of stem cells. Cell fusion, an essential physiological process to establish and expand the STB, has recently been recognized to be a further trigger of cell senescence. Senescence as a response to fusion may have evolved to halt the proliferation of cells infected with fusogenic viruses (e.g. the measles virus) so it is of note that cytotrophoblast fusion requires a retroviral fusogen Syncytin-1 (a product of the ERVW-1 gene). Examples of markers of SASP include but are not limited to IL-6, IL-8, IL-1alpha, IL-1beta, CCL2, TIMP1, TIMP2, MCP1, MIP3.

In some embodiments, the endogenous biomarker is a marker of deregulated nutrient-sensing such as insulin resistance and calorie restriction. Examples of deregulated nutrient-sensing markers include but are not limited to IGF-1, PI3K/AKT/mTOR pathway, AMPK, PGC1alpha, NAD+/NADH, SIRT and FOXO.

In some embodiments, the endogenous biomarker is a marker of chronic inflammation. Inflammation is known to be important in aging and age-related diseases, and is sometimes suggested as a principal aging mechanism. Examples of inflammation markers include but are not limited to TNF, IL-6, IL-8, IL-10, IL-12, IL-18, hsCRP, IFN-γ, IL-1β, IL-IRA, MCP, MIP, SGP130, STNF-RI, STNF-RII, TGF-β1, and TRAIL. In some embodiments, the marker of inflammation is a pro-inflammatory cytokine, such as IL-6, IL-8, IL-10, IL-12, and IL-18.

The biomarker can also be a marker of cell cycle. The cell cycle is a sequence of coordinated events which lead to cell division, critical for both development and viability of multicellular organisms. A stable cell cycle arrest which marks an inability of the cell to continue dividing is an indispensable and one of the defining features of senescent cells. One example of cell cycle markers is Ki67, a nuclear protein associated with cellular proliferation and ribosomal RNA transcription, and is routinely used as a marker of proliferating cells.

The biomarker can also be a marker of loss of proteostasis. Aging cells accumulate damaged and misfolded proteins through a functional decline in their protein homeostasis (proteostasis) machinery, leading to reduced cellular viability and the development of protein misfolding diseases.

The biomarker can also be a marker of organelle dysfunction, such as mitochondria dysfunction, proteosome dysfunction, endoplasmic reticulum dysfunction, Golgi apparatus dysfunction, and nuclear envelope dysfunction.

The biomarker can also be a marker of disabled macroautophagy. Macroautophagy is a catabolic process in which portions of the cytoplasm are sequestered within double- or multimembraned vesicles termed autophagosomes and then delivered to lysosomes for bulk degradation. Macroautophagy has been shown to decrease with age.

The biomarker can also be a marker of altered intercellular communication. Intercellular communication refers to the various ways and structures that biological cells use to communicate with each other directly or through their environment. Different types of cells use different proteins and mechanisms to communicate with one another using extracellular signaling molecules. Altered intercellular communication is considered a hallmark of aging, and might affect 1) the canonical senescence associated secretory phenotype, 2) direct cell-cell communication through gap junctions or tubule-like structures and 3) long distance communication, involving extracellular vesicles and paracrine communication mediated by Connexin-containing hemichannels.

The biomarker can also be a marker of stem cell exhaustion. The functional stem cell pool is usually depleted in aged animals. Depletion of the stem cell pool with age may occur because these cells lose self-renewal activity and terminally differentiate, thereby exiting the stem cell pool, or because they undergo apoptosis or senescence induced by exposure to cellular stress,

Indicators of the mTOR/AMPK pathway might also be suitable biomarkers to be used in the method disclosed herein. The mammalian/mechanistic target of rapamycin (mTOR) is a key component of cellular metabolism that integrates nutrient sensing with cellular processes that fuel cell growth and proliferation. mTOR receives and integrates inputs from its upstream regulators, one of which is the energy status sensitive AMPK pathway.

### III. Method of Assessing Aging

The present application in one aspect provides a method of assessing an aging process of a mammalian cell, comprising subjecting a stem cell (such as a totipotent or a pluripotent stem cell) or an early extraembryonic cell to a condition that allows differentiation of the stem cell or extraembryonic cell towards a STB, and determining one or more characteristics of differentiation of stem cell or the early extraembryonic cell.

In some embodiments, the method of assessing an aging process comprises subjecting a stem cell to a condition that allows differentiation of the stem cell towards a STB. In some embodiments, the method of assessing an aging process comprises subjecting an early extraembryonic cell to a condition that allows differentiation of the early extraembryonic cell towards a STB. In some embodiments, the stem cell is a totipotent stem cell or a pluripotent stem cell. In some embodiments, the early extraembryonic cell is a trophoblast stem cell (“TSC”) or a trophoblast progenitor cell (“TPC”). In some embodiments, the early extraembryonic cell is a TSC. In some embodiments, the early extraembryonic cell is derived from a totipotent stem cell, a pluripotent stem cell, an embryo tissue, or a placenta tissue. In some embodiments, the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

1. Culturing Conditions that Allow STB Differentiation

Cultured EPSCs can be induced to differentiate towards STBs. In some embodiments, EPSCs are contacted with Y27632 and SB431542. Y27632 is a cell-permeable, highly potent and selective inhibitor of Rho-associated, coiled-coil containing protein kinase. SB431542 is TGF-beta inhibitor. In one particular embodiment, human EPSCs are dissociated with TrypLE and seeded in 100× Geltrex coated six-well plates at a density of 1×10⁵ cells per well. Cells are cultured (pre-treatment) in 20% KSR media supplemented with 10 μM Y27632 for one day. From the second day, 10 μM SB431542 is added into 20% KSR media to start the differentiation. STB features can be detected around day 7 or day 8.

EPSCs and other stem cells can be induced to differentiate into TSCs, and the TSCs can then be induced to differentiate into STBs. Methods for inducing EPSCs to differentiate towards TSCs are described in Section II. 2. Methods for inducing TSCs to differentiate towards STBs are known in the art (Sec, e.g., US20230220334A1). In some embodiments, the condition for STB differentiation comprises a cell culture medium comprising DMEM/F12, β-mercaptoethanol, Penicillin-Streptomycin, 7.5% BSA, ITS-X, Y27632, Forskolin, and KnockOut Serum Replacement. In some embodiments, TSCs are cultured in syncytiotrophoblasts medium (STBM) for around 6 days. The STBM comprises: DMEM/F12 supplemented with 110 μM β-mercaptoethanol, 0.5% Penicillin-Streptomycin, 0.3% BSA, 1×ITS-X, 2.5 μM Y27632, 2 μM Forskolin, and 4% KnockOut Serum Replacement. The cells become early syncytiotrophoblasts on day 2-3, and mature syncytiotrophoblasts around day 6.

### 2. Characteristics of Differentiation

In some embodiments, the method comprises determining one or more characteristics of differentiation of stem cell or the early extraembryonic cell. Characteristics of differentiation include but are not limited to: 1) presence or absence of a biomarker associated with differentiation of the early extraembryonic cell; 2) level of a biomarker associated with differentiation of the early extraembryonic cell; 3) secretion of a biomarker associated with differentiation of the early extraembryonic cell; 4) cell morphology; 5) rate of change to a differentiated state; 6) property of a cellular organelle; and 7) number of nuclei in the cell; and 8) the presence or absence of a reporter molecule.

In some embodiments, the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9mc3, H3K4mc3, H3K27mc3, H3K9ac3, SETDB1, KAP1, LaminA, LaminB1, LaminC, HP1γ, HP1α, LTR5, pTBK1, LINE1, HERVK, SIRT family members and combinations thereof and combinations thereof. Without wishing to be bound by any theory, genes such as OCT4, NANOG, SSEA-4, SOX2, REX1, and SALL4 might be considered as markers for Expanded Potential Stem Cells (EPSCs); genes such as SDC1, KLF5, TP63, TEAD4, TBX3, KRT7, GATA2, and GATA3 might be considered as markers for TSCs; and genes such as CGB, CSH1, CGA, and ERVW1 might be considered as markers for STBs.

In some embodiments, the characteristics of differentiation comprises presence or absence of a biomarker associated with differentiation of the early extraembryonic cell. In some embodiments, the biomarker is selected from the group consisting of OCT4, NANOG, SSEA-4, SOX2, REX1, and SALL4. In some embodiments, the characteristics of differentiation comprises level of a biomarker associated with differentiation of the early extraembryonic cell. In some embodiments, the biomarker is selected from the group consisting of SDC1, KLF5, TP63, TEAD4, TBX3, KRT7, GATA2, GATA3, CGB, CSH1, CGA, and ERVW1.

In some embodiments, the biomarker is a hormone. Syncytiotrophoblasts secrets large amounts of hormones including metabolic proteins (leptin, adiponectin), peptide hormones (hCG, hPL, PGH) and steroid hormones (progesterone, estrogens). Human chorionic gonadotropin hormone (hCG) is comprised of α- and β-subunits, and β-hCG can be measured by ELISA. In some embodiments, the biomarker is Human chorionic gonadotropin (hCG). In some embodiments, the biomarker is β-hCG.

The biomarkers can be detected or measured at the mRNA level or the protein level. Various methods are available for mRNA level detection/measurement, including RNA sequencing, RT-qPCR, and in situ hybridization. Methods for protein level detection/measurement include western blot, ELISA, proteomics, and immunofluorescence.

Aside from presence/absence, level, and secretion of a biomarker, the characteristic of differentiation also can be cell morphology. Cell morphological properties such as cell volume, area, and thickness can be determined using microscopy. TSC cells are characterized as mononucleated epithelial cells with well-demarcated cell borders and large nuclei, while STB cells are featured with villous multinucleated entities.

In some embodiments, the characteristic of differentiation is assessed as the rate of change to a differentiated state. In one particular embodiment, the cells become early syncytiotrophoblasts around day 2-3, and mature syncytiotrophoblasts around day 6. The rate of change to a differentiated state can be determined as the amount of time it takes for the cells to become early syncytiotrophoblasts and/or mature syncytiotrophoblasts in comparison to 2-3 days and 6 days, respectively. The rate of change can be represented as percent change, such as about 10% faster/slower, about 20% faster/slower, about 30% faster/slower, about 40% faster/slower, about 50% faster/slower, about 60% faster/slower, about 70% faster/slower, about 80% faster/slower, about 90% faster/slower, about 100% faster/slower, etc.

In some embodiments, the characteristics of differentiation include a property of a cellular organelle. In some embodiments, the cellular organelle is selected from the group consisting of: mitochondrion, proteosome, endoplasmic reticulum, Golgi apparatus, and nuclear envelope. In some embodiments, the cellular organelle is mitochondrion. As the stem cell or the early extraembryonic cell differentiate towards STB, properties (including the number, morphology, and function) of mitochondria are expected to change. Mitophagy is expected to increase. The properties can be studied using methods such as mitochondrial labeling and/or electric microscopy imaging. Accumulated damaged and misfolded proteins in senescence cells result from a functional decline in the proteosomes, which can be marked by E3 ubiquitin ligases and chaperone family heat shock proteins (HSP) dysfunction. The endoplasmic reticulum (ER) and Golgi apparatus are both part of the membranous tubular network that plays a major role in ions homeostasis, lipid, and protein biosynthesis. Together with proteosomes, they maintain the protein homeostasis (also called proteostasis). Due to different kind of stress such as nutrient deprivation, viral infection and hypoxia, unfolded or misfolded proteins can accumulate and aggregate in these organelles, and hence become detrimental to cell survival. ER stress activate ER transmembrane proteins PKR-like ER kinase (PERK), IRE1α, and ATF6α. Golgi apparatus is important for the bidirectional vesicular trafficking among the ER, proteosomes and mitochondria. Molecules related to Golgi apparatus include Nir2, CERT, and oxysterol-binding protein (OSBP).

In some embodiments, the characteristics of differentiation include number of nuclei in the cell. STBs are multinucleated, and the number of nuclei is not consistent in all the STB cells. The number of nuclei can be determined by DAPI or Hoechst staining and quantified with confocal microscope.

In some embodiments, the characteristics of differentiation include the presence or absence of a reporter molecule, as described in Section II. 1.

### IV. Method of Evaluating and Screening for a Candidate Agent

One aspect of the disclosed invention provides a method of evaluating anti-aging function of a candidate agent, comprising: 1) subjecting a stem cell (such as a totipotent or a pluripotent stem cell) or an early extraembryonic cell to a condition that allows differentiation of the stem cell or extraembryonic cell towards a trophoblast, specifically STB; 2) contacting the stem cell or early extraembryonic cell with the candidate agent before, during, or after subjecting the stem cell or early extraembryonic cell to a condition that allows the differentiation; and 3) assessing change of one or more characteristics of differentiation of the stem cell or early extraembryonic cell relative to a stem cell or an early extraembryonic cell without contacting with the candidate agent. In some embodiments, the candidate agent is selected from the group consisting of: an antibody, a virus, a virus-like, a small molecule, a peptide, a polypeptide, a DNA, an mRNA, a guide RNA, a microRNA, an RNAi, a LncRNA, an siRNA molecule, and an antisense RNA. In some embodiments, the candidate agent is a naturally occurring substance. In some embodiments, the candidate agent is a nutraceutical.

One aspect of the disclosed invention provides a method of screening for a candidate agent having an anti-aging function, comprising: 1) evaluating the anti-aging function of a plurality of candidate agents; and 2) identifying the candidate agent having an anti-aging function based on the ability of the candidate agent to cause change of one or more characteristics of differentiation of the stem cell or early extraembryonic cell relative to a stem cell or early extraembryonic cell without the candidate agent. In some embodiments, the candidate agent is selected from the group consisting of: an antibody, a virus, a virus-like, a small molecule, a peptide, a polypeptide, a DNA, an mRNA, a guide RNA, a microRNA, an RNAi, a LncRNA, an siRNA molecule, and an antisense RNA. In some embodiments, the candidate agent is a naturally occurring substance. In some embodiments, the candidate agent is a nutraceutical.

### 1. Evaluation of Anti-Aging Functions

In some embodiments, the method of evaluating anti-aging function of a candidate agent comprises subjecting a stem cell (such as a totipotent or a pluripotent stem cell) or an early extraembryonic cell to a condition that allows differentiation of the stem cell or extraembryonic cell towards a trophoblast, specifically STB as described in Section III.1. In some embodiments, the stem cell or early extraembryonic cell is contacted with the candidate agent before being subjected to the condition that allows the differentiation. In some embodiments, the stem cell or early extraembryonic cell is contacted with the candidate agent simultaneously with being subjected to the condition that allows the differentiation. In some embodiments, the stem cell or early extraembryonic cell is contacted with the candidate agent after being subjected to the condition that allows the differentiation. For example, in some embodiments, the stem cell or early extraembryonic cell is subjected to the condition that allows the differentiation on day 0, and is contacted with the candidate agent simultaneously. In some embodiments, the stem cell or early extraembryonic cell is subjected to the condition that allows the differentiation on day 0, and is contacted with the candidate agent on day 2. In some embodiments, the stem cell or early extraembryonic cell is contacted with the candidate agent on day 0, and is subjected to the condition that allows the differentiation on day 2.

In some embodiments, the method of evaluating anti-aging function of a candidate agent comprises assessing change of one or more characteristics of differentiation of the stem cell or early extraembryonic cell relative to a stem cell or an early extraembryonic cell without contacting with the candidate agent. Characteristics of differentiation and methods of detection and/or measurement are described in Section III. 2. In some embodiments, the characteristics of differentiation are evaluated at least one day after subjecting the stem cell or early extraembryonic cell to a condition for differentiation. In some embodiments, the characteristics of differentiation are evaluated 2 to 8 days after subjecting the stem cell or early extraembryonic cell to a condition for differentiation.

In some embodiments, the characteristics of differentiation are evaluated at least one day after subjecting the stem cell or early extraembryonic cell to a condition for differentiation. In some embodiments, the characteristics of differentiation are evaluated 2 to 8 days after subjecting the stem cell or early extraembryonic cell to a condition for differentiation. An agent that increase or decrease a differentiation or senescence related marker signal compared to a stem cell or early extraembryonic cell that has not been in contact the agent by for about or more than 20% can be considered as having anti-ageing effect. The agent can then be subject to further validation and mechanisms investigation.

In some embodiments, evaluating anti-aging function of the candidate agent comprises assessing whether the candidate agent has anti-aging function. In some embodiments, evaluating anti-aging function of the candidate agent comprises determining an effective concentration of the candidate agent having an anti-aging function. In some embodiments, the method might further comprise contacting the stem cell or early extraembryonic cell with the candidate agent at different concentrations.

In one embodiment, the method of evaluating anti-aging function of a candidate agent comprises 1) subjecting a TSC to a condition that allows differentiation of the TSC towards a STB; 2) contacting the TSC with the candidate agent simultaneously with subjecting the TSC to a condition that allows the differentiation; and 3) assessing change of expression of hCG and/or differentiation/senescence markers of the STB relative to a STB without contacting with the candidate agent. If the cells that have contacted the candidate agent secrets less hCG and/or differentiation/senescence markers compared to a cell without contacting with the candidate agent, it might be inferred that the candidate agent has anti-aging function.

In one embodiment, the method of evaluating anti-aging function of a candidate agent comprises 1) subjecting TSC to a condition that allows differentiation of the TSC towards a STB; 2) contacting the TSC with the candidate agent at different concentrations simultaneously with subjecting the TSC to a condition that allows the differentiation; and 3) assessing change of expression of hCG and/or differentiation/senescence markers of the STB with and without contacting with the candidate agent. The effective concentration of the candidate agent might be determined as the lowest concentration at which expression of hCG and/or differentiation/senescence markers is decreased by at least 20% compared to the STB without contacting with the candidate agent.

### 2. Evaluation of Effects on Viability

In some embodiments, the method further comprises determining the effect of the candidate agent on the viability of the stem cell or early extraembryonic cell, or cells differentiated therefrom. Methods for assaying cell viability are known in the art, including MTT assay, tetrazolium reduction, resazurin reduction, protease markers, and ATP detection. Cell viability can also be assessed through intensity/cell count quantification with confocal microscope imaging of Hoechst stained live cells or fixed attached cells

### 3. Exemplary Agent Screening Workflow

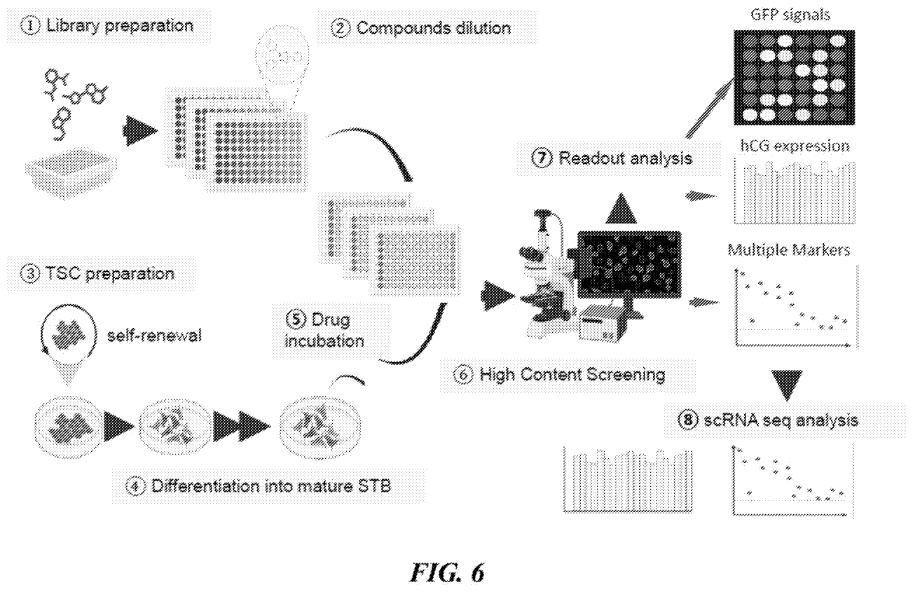

The present invention provides a method for agent screening for potential anti-aging effects. In an exemplary workflow laid out in FIG. 6, the method comprises: 1) agent library preparation; 2) compound dilution; 3) TSC preparation; 4) TSC-STB differentiation; 5) high content microscopy screening; 7) readout analyses such as signals (fluorescence and/or non-fluorescence) of reporter molecules, hCG expression, biomarker analysis, and 9) single cell RNA sequencing (scRNA seq).

### V. Method of Identifying a Candidate Gene

### 1. Modified Cells

In one aspect, disclosed herein is a method of identifying a candidate gene involved in an aging process, comprising: 1) subjecting a plurality of stem cells (such as totipotent or pluripotent stem cells) or early extraembryonic cells to a condition that allows differentiation of the plurality of stem cells or early extraembryonic cells towards STBs, wherein each of the plurality of the stem cells or early extraembryonic cells comprises an alteration in a candidate gene relative to a wildtype stem cell or early extraembryonic cell, wherein at least two of the plurality of stem cells or early extraembryonic cells contain different alterations; 2) determining one or more characteristics of differentiation of the stem cells or early extraembryonic cells; and 3) identifying the candidate gene involved in an aging process based on the ability of the alteration in the candidate gene to cause change of one or more characteristics of differentiation of the stem cell or early extraembryonic cell relative to a stem cell or an early extraembryonic cell without the corresponding alternation. In some embodiments, the stem cell or early extraembryonic cell is genetically modified.

Suitable methods for genetically modifying the stem cell or early extraembryonic cell are known in the art, including clustered regularly interspaced short palindromic repeats (CRISPR)-CRISPR-associated protein (Cas), transcription activator-like effector nucleases (TALENs), zinc-finger nucleases (ZFNs), homing endonucleases or meganucleases, and base editing. The outcomes of the genetic modification might be gene knock-out/knock-in, gene mutations, or gene inversion.

CRISPR is a family of DNA sequences found in the genomes of prokaryotic organisms such as bacteria and archaea. These sequences are derived from DNA fragments of bacteriophages that had previously infected the prokaryote. They are used to detect and destroy DNA from similar bacteriophages during subsequent infections. CRISPR-Cas systems are composed of CRISPR repeat-spacer arrays, which can be further transcribed into CRISPR RNA (crRNA) and trans-activating CRISPR RNA (tracrRNA), and a set of CRISPR-associated (cas) genes which encode Cas proteins with endonuclease activity. CRISPR-Cas systems can be classified into 2 classes (Class 1 and Class 2), 6 types (I to VI) and several subtypes, with multi-Cas protein effector complexes in Class 1 systems (Type I, III, and IV) and a single effector protein in Class 2 systems (Type II, V, and VI). Type II CRISPR-Cas9 system derived from Streptococcus pyogenes (SpCas9) is one of the best characterized and most commonly used category in numerous CRISPR-Cas systems. The main components of CRISPR-Cas9 system are RNA-guided Cas9 endonuclease and a single-guide RNA (sgRNA). The Cas9 protein possesses two nuclease domains, named HNH and RuvC, and each cleaves one strand of the target double-stranded DNA. A single-guide RNA (sgRNA) is a simplified combination of crRNA and tracrRNA. The Cas9 nuclease and sgRNA form a Cas9 ribonucleoprotein (RNP), which can bind and cleave the specific DNA target. Furthermore, a protospacer adjacent motif (PAM) sequence is required for Cas9 protein's binding to the target DNA.

ZFNs are fusions between a custom-designed Cys2-His2 zinc-finger protein and the cleavage domain of the FokI restriction endonuclease. ZFNs function as dimers, with each monomer recognizing a specific “half site” sequence—typically nine to 18 base pairs (bps) of DNA—via the zinc-finger DNA-binding domain.

TALENs are structurally similar to ZFNs. Both methods use the Fokl nuclease to cut DNA and require dimerization to function, however, the DNA binding domains differ. TALENs use transcription activator-like effectors (TALEs), tandem arrays of 33-35 amino acid repeats. The amino acid repeats possess single-nucleotide recognition, thereby increasing targeting capabilities and specificity compared to ZFNs.

Homing endonucleases, also known as meganucleases are a collection of naturally occurring enzymes that recognize and cleave long DNA sequences (14-40 bps). These enzymes make extensive sequence-specific contacts with their DNA substrate and thus typically show exquisite specificity.

Base editing is a relatively new method of genome editing derived from CRISPR-Cas9. Unlike traditional CRISPR systems, base editors (BEs) do not induce double-stranded breaks in the genome. Base editing systems, use a ‘catalytically dead’ Cas9 (dCas9), which cannot cleave DNA, fused to bacterial enzymes called DNA deaminases. Cytidine deaminases, which induce C to T substitutions, are naturally occurring in bacteria, while adenine deaminases, which induce A to G substitutions, were engineered from bacterial enzymes specifically for base editing purposes. Fusing dCas9 to either a cytidine deaminase (CBEs) or an adenine deaminase (ABEs) and providing a sgRNA to direct it to the target sequence, allows researchers to introduce substitutions in DNA.

The invention will now be described by reference to the following examples which are meant to be illustrative of embodiments of the invention and are not to be construed as limiting the invention.

### Exemplary Embodiments

Embodiment 1. A mammalian cellular aging evaluation system comprising a totipotent or pluripotent stem cell or an early extraembryonic cell, wherein the totipotent or pluripotent stem cell or early extraembryonic cell optionally comprises a reporter molecule or a heterologous nucleic acid encoding a reporter molecule that is indicative of senescence or differentiation of the totipotent or pluripotent stem cell or early extraembryonic cell towards a trophoblast or syncytiotrophoblast (“STB”).

Embodiment 2. The mammalian cellular aging evaluation system of Embodiment 1, wherein the totipotent or pluripotent stem cell or early extraembryonic cell is naturally occurring.

Embodiment 3. The mammalian cellular aging evaluation system of Embodiment 1, wherein the totipotent or pluripotent stem cell or early extraembryonic cell is genetically modified.

Embodiment 4. The mammalian cellular aging evaluation system of any one of Embodiments 1-3, wherein the totipotent or pluripotent stem cell or early extraembryonic cell comprises a reporter molecule.

Embodiment 5. The mammalian cellular aging evaluation system of Embodiment 4, wherein the reporter molecule is selected from the group consisting of: a physically activated molecule and a chemically activated molecule.

Embodiment 6. The mammalian cellular aging evaluation system of any one of Embodiments 1-3, wherein the totipotent or pluripotent stem cell or early extraembryonic cell comprises the heterologous nucleic acid.

Embodiment 7. The mammalian cellular aging evaluation system of Embodiment 6, wherein the heterologous nucleic acid is integrated into the genome of the totipotent or pluripotent stem cell or early extraembryonic cell.

Embodiment 8. The mammalian cellular aging evaluation system of Embodiment 7, wherein the heterologous nucleic acid is under the control of a promoter of an endogenous biomarker gene encoding a biomarker indicative of senescence or differentiation of the totipotent or pluripotent stem cell or early extraembryonic cell towards a trophoblast or STB.

Embodiment 9. The mammalian cellular aging evaluation system of Embodiment 8, wherein the heterologous nucleic acid is introduced into the totipotent or pluripotent stem cell or early extraembryonic cell by a gene editing tool.

Embodiment 10. The mammalian cellular aging evaluation system of any one of Embodiments 6-9, wherein the reporter molecule is selected from the group consisting of Green Fluorescent Protein (GFP), Red Fluorescent Protein (RFP), Yellow Fluorescent Protein (YFP), mCherry, tdTomato, photoconvertible fluorescent proteins, bioluminescence, enzyme assay, antibody-based assays, chloramphenicol acetyltransferase, and biosensors.

Embodiment 11. The mammalian cellular aging evaluation system of any one of Embodiments 1-10, wherein the system comprises an early extraembryonic cell.

Embodiment 12. The mammalian cellular aging evaluation system of Embodiment 11, wherein the early extraembryonic cell is a trophoblast stem cell (“TSC”) or a trophoblast progenitor cell (“TPC”).

Embodiment 13. The mammalian cellular aging evaluation system of Embodiment 12, wherein the early extraembryonic cell is a TSC.

Embodiment 14. The mammalian cellular aging evaluation system of any one of Embodiments 11-13, wherein the early extraembryonic cell is derived from a totipotent stem cell, a pluripotent stem cell, an embryo tissue, or a placenta tissue.

Embodiment 15. The mammalian cellular aging evaluation system of any one of Embodiments 11-14, wherein the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

Embodiment 16. The mammalian cellular aging evaluation system of any one of Embodiments 8-15, wherein the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. and stem cell exhaustion.

Embodiment 17. The mammalian cellular aging evaluation system of Embodiment 16, wherein the endogenous biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9mc3, H3K4mc3, H3K27me3, H3K9ac3, SETDB1, KAP1, LaminA, LaminB1, LaminC, HP1γ, HP1α, LTR5, pTBK1, LINE1, HERVK, and SIRT family members.

Embodiment 18. The mammalian cellular aging evaluation system of any one of Embodiments 1-10, wherein the system comprises a totipotent or pluripotent stem cell.

Embodiment 19. The mammalian cellular aging evaluation system of Embodiment 18, wherein the system comprises a totipotent stem cell.

Embodiment 20. The mammalian cellular aging evaluation system of Embodiment 18, wherein the system comprises a pluripotent stem cell.

Embodiment 21. The mammalian cellular aging evaluation system of any one of Embodiments 1-20, wherein the totipotent or pluripotent stem cell or early extraembryonic cell is derived from a human, a pig, a cow, a mouse, a rat, a bat, a rabbit, a dog, a cat, and a sheep.

Embodiment 22. A method of assessing an aging process of a mammalian cell, comprising subjecting a totipotent or pluripotent stem cell or an early extraembryonic cell to a condition that allows differentiation of the totipotent or pluripotent stem cell or extraembryonic cell towards a trophoblast or STB, and determining one or more characteristics of differentiation of the totipotent or pluripotent stem cell or the early extraembryonic cell.

Embodiment 23. A method of evaluating anti-aging function of a candidate agent, comprising: 1) subjecting a totipotent or pluripotent stem cell or an early extraembryonic cell to a condition that allows differentiation of the totipotent or pluripotent stem cell or early extraembryonic cell towards a trophoblast, specifically STB; 2) contacting the totipotent or pluripotent stem cell or early extraembryonic cell with the candidate agent before, during, or after subjecting the totipotent or pluripotent stem cell or early extraembryonic cell to a condition that allows the differentiation; and 3) assessing change of one or more characteristics of differentiation of the totipotent or pluripotent stem cell or early extraembryonic cell relative to a totipotent or pluripotent stem cell or an early extraembryonic cell without contacting with the candidate agent.

Embodiment 24. The method of Embodiment 23, wherein evaluating anti-aging function of the candidate agent comprises assessing whether the candidate agent has anti-aging function.

Embodiment 25. The method of Embodiment 23, wherein evaluating anti-aging function of the candidate agent comprises determining an effective concentration of the candidate agent having an anti-aging function.

Embodiment 26. A method of screening for a candidate agent having an anti-aging function, comprising: 1) evaluating the anti-aging function of a plurality of candidate agents according to the method of any one of Embodiments 23-25; and 2) identifying the candidate agent having an anti-aging function based on the ability of the candidate agent to cause change of one or more characteristics of differentiation of the totipotent or pluripotent stem cell or early extraembryonic cell to a trophoblast or STB relative to a totipotent or pluripotent stem cell or early extraembryonic cell without the candidate agent.

Embodiment 27. The method of any one of Embodiments 23-26, wherein the candidate agent is selected from the group consisting of: an antibody, a virus, a virus-like, a small molecule, a peptide, a polypeptide, a DNA, an mRNA, a guide RNA, a microRNA, an RNAi, a LncRNA, an siRNA molecule, and an antisense RNA.

Embodiment 28. The method of any one of Embodiments 23-26, wherein the candidate agent is a naturally occurring substance.

Embodiment 29. The method of any one of Embodiments 23-26, wherein the candidate agent is a nutraceutical.

Embodiment 30. A method of identifying a candidate gene involved in an aging process, comprising: 1) subjecting a plurality of totipotent or pluripotent stem cells or early extraembryonic cells to a condition that allows differentiation of the plurality of totipotent or pluripotent stem cells or early extraembryonic cells towards trophoblasts or STBs, wherein each of the plurality of totipotent or pluripotent stem cells or early extraembryonic cells comprises an alteration in a candidate gene relative to a wildtype totipotent or pluripotent stem cell or early extraembryonic cell, wherein at least two of the plurality of totipotent or pluripotent stem cells or early extraembryonic cells contain different alterations; 2) determining one or more characteristics of differentiation of the plurality of totipotent or pluripotent stem cells or early extraembryonic cells; and 3) identifying the candidate gene involved in an aging process based on the ability of the alteration in the candidate gene to cause change of one or more characteristics of differentiation of the plurality of totipotent or pluripotent stem cell or early extraembryonic cell relative to a totipotent or pluripotent stem cell or an early extraembryonic cell without the corresponding alternation.

Embodiment 31. The method of any one of Embodiments 22-30, wherein the totipotent or pluripotent stem cell or early extraembryonic cell is naturally occurring.

Embodiment 32. The method of any one of Embodiments 22-30, wherein the totipotent or pluripotent stem cell or early extraembryonic cell is genetically modified.

Embodiment 33. The method of any one of Embodiments 22-32, wherein the totipotent or pluripotent stem cell or early extraembryonic cell comprises a reporter molecule that is indicative of differentiation of the totipotent or pluripotent stem cell or early extraembryonic cell towards an STB.

Embodiment 34. The method of Embodiment 33, wherein the reporter molecule is selected from the group consisting of: a physically activated molecule and a chemically activated molecule.

Embodiment 35. The method of any one of Embodiments 22-32, wherein the totipotent or pluripotent stem cell or early extraembryonic cell comprises a heterologous nucleic acid encoding a reporter molecule that is indicative of differentiation of the totipotent or pluripotent stem cell or early extraembryonic cell towards a trophoblast or STB.

Embodiment 36. The method of Embodiment 35, wherein the heterologous nucleic acid is integrated into the genome of the totipotent or pluripotent stem cell or early extraembryonic cell.

Embodiment 37. The method of Embodiment 36, wherein the heterologous nucleic acid is under the control of a promoter of an endogenous biomarker gene encoding a biomarker indicative of differentiation of the totipotent or pluripotent stem cell or early extraembryonic cell towards an STB.

Embodiment 38. The method of any one of Embodiments 35-37, wherein the heterologous nucleic acid is introduced into the totipotent or pluripotent stem cell or early extraembryonic cell by a gene editing tool.

Embodiment 39. The method of any one of Embodiments 35-38, wherein the reporter molecule is selected from the group consisting of Green Fluorescent Protein (GFP), Red Fluorescent Protein (RFP), Yellow Fluorescent Protein (YFP), mCherry, and tdTomato.

Embodiment 40. The method of any one of Embodiments 22-39, wherein the STB is an early STB, a late STB, or a mature and aged STB.

Embodiment 41. The method of any one of Embodiments 22-40, wherein the one or more characteristics of differentiation comprises: 1) presence or absence of a biomarker associated with differentiation of the totipotent or pluripotent stem cell or early extraembryonic cell; 2) level of a biomarker associated with differentiation of the totipotent or pluripotent stem cell or early extraembryonic cell; 3) secretion of a biomarker associated with differentiation of the totipotent or pluripotent stem cell or early extraembryonic cell; 4) cell morphology; 5) rate of change to a differentiated state; 6) property of a cellular organelle; and 7) number of nuclei in the cell; and 8) the presence or absence of a reporter molecule.

Embodiment 42. The method of Embodiment 41, wherein the one or more characteristics of differentiation comprises property of a cellular organelle.

Embodiment 43. The method of Embodiment 42, wherein the cellular organelle is selected from the group consisting of: mitochondria, proteosome, endoplasmic reticulum, Golgi apparatus, and nuclear envelope.

Embodiment 44. The method of Embodiment 43, wherein the property of the cellular organelle comprises number, morphology, and function of the organelle.

Embodiment 45. The method of any one of Embodiments 22-44, wherein the totipotent or pluripotent stem cell or early extraembryonic cell is an early extraembryonic cell.

Embodiment 46. The method of Embodiment 45, wherein the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

Embodiment 47. The method of Embodiment 41, wherein the one or more characteristics of differentiation comprises presence or absence of a biomarker associated with differentiation of the early extraembryonic cell.

Embodiment 48. The method of Embodiment 47, wherein the biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. and stem cell exhaustion.

Embodiment 49. The method of Embodiment 48, wherein the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, SIRT family members and combinations thereof.

Embodiment 50. The method of any one of Embodiments 47-49, wherein the one or more characteristics of differentiation comprises molecular indicators of nucleotide excision repair (NER), base-excision repair (BER), DNA mismatch repair (MMR), Fanconi anemia pathway, homologous recombination (HR), nonhomologous end joining (NHEJ), variant histones, insulin resistance, pre-inflammation factors, the mTOR/AMPK pathway, mitophagy, senescence-associated secretory phenotype, and/or senescence.

Embodiment 51. The method of any one of Embodiments 22-44, wherein the totipotent or pluripotent stem cell or early extraembryonic cell is a totipotent or pluripotent stem cell.

Embodiment 52. The method of Embodiment 51, wherein the totipotent or pluripotent stem cell is a totipotent stem cell.

Embodiment 53. The method of Embodiment 51, wherein the totipotent or pluripotent stem cell is a pluripotent stem cell.

Embodiment 54. The method of any one of Embodiments 51-53, wherein the one or more characteristics of differentiation comprises presence or absence of a biomarker associated with differentiation of the totipotent or pluripotent stem cell.

Embodiment 55. The method of Embodiment 54, wherein the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, SIRT family members and combinations thereof.

Embodiment 56. The method of any one of Embodiments 47-50 and 54-55, wherein the biomarker is a RNA molecule.

Embodiment 57. The method of Embodiment 56, wherein assessing change of one or more characteristics of differentiation comprises RNA sequencing, RT-qPCR, and/or in situ hybridization.

Embodiment 58. The method of any one of Embodiments 47-50 and 54-55, wherein the biomarker is a protein molecule.

Embodiment 59. The method of Embodiment 58, wherein assessing change of one or more characteristics of differentiation comprises western blot, ELISA, proteomics, and/or immunofluorescence.

Embodiment 60. The method of any one of Embodiments 23-29 and 31-59, wherein the totipotent or pluripotent stem cell or early extraembryonic cell is contacted with the candidate agent prior to being subjected to a condition for differentiation.

Embodiment 61. The method of any one of Embodiments 23-29 and 31-59, wherein the totipotent or pluripotent stem cell or early extraembryonic cell is contacted with the candidate agent simultaneously with being subjected to a condition for differentiation.

Embodiment 62. The method of any one of Embodiments 23-29 and 31-59, wherein the totipotent or pluripotent stem cell or early extraembryonic cell is contacted with the candidate agent after being subjected to a condition for differentiation.

Embodiment 63. The method of any one of Embodiments 22-62, wherein the condition for differentiation comprises a cell culture medium comprising DMEM/F12, β-mercaptoethanol, Penicillin-Streptomycin-Glutamine, BSA, ITS-X, Y27632, Forskolin, and KnockOut Serum Replacement.

Embodiment 64. The method of any one of Embodiments 22-63, wherein the characteristics of differentiation are evaluated at least one day after subjecting the totipotent or pluripotent stem cell or early extraembryonic cell to a condition for differentiation.

Embodiment 65. The method of Embodiment 64, wherein the characteristics of differentiation are evaluated 2 to 8 days after subjecting the totipotent or pluripotent stem cell or early extraembryonic cell to a condition for differentiation.

Embodiment 66. The method of any one of Embodiments 22-65, wherein the totipotent or pluripotent stem cell or early extraembryonic cell is derived from a human, a pig, a cow, a mouse, a rat, a bat, a rabbit, a dog, a cat, and a sheep.

Embodiment 67. The method of any one of Embodiments 23-29 and 31-66, further comprising determining the effect of the candidate agent on the viability of the totipotent or pluripotent stem cell or early extraembryonic cell, or cells differentiated therefrom.

### EXAMPLES

### Example 1: Methods

### 1.1 Cell Lines

Expanded Potential Stem Cells (EPSCs) were derived from somatic cell lines, embryonic stem cells or other stem cell lines. SNL 76/7 was established by Dr. Allan Bradley, clonally derived from a mouse fibroblast STO cell line transformed with neomycin resistance and murine LIF genes. The cells were proliferated and treated with γ-irradiation to be used as feeder cells for EPSC culturing. Reporter cell lines were built based on genome editing on normal EPSCby certain constructs transfection and purification. TSC cell lines were generated from different EPSC cell lines via differentiating with TSC medium (TSCM) and single colony picking. Reporter TSC cell lines were differentiated from reporter EPSCs. STB cells were differentiated from TSC cell lines with STB medium (STBM). All cells were incubated at 37° C. in a humidified incubator with 5% CO₂, and routinely tested for mycoplasma every week using PlasmoTest (InvivoGen).

### 1.2 Cell Culture

Human EPSC cells were maintained on SNL 76/7 feeder layers with hEPSC Medium (EPSCM), and passaged at a ratio 1:5-1:10 with TrypLE™ Express Enzyme (1×). The hEPSCM was a N2B27-based media supplemented with 4 small molecules: 2.5 μM XAV939, 2.5 μM Endo-IWR-1, 1 μM CHIR99021 and 0.1 μM A419259. The components of N2B27-based medium is published in Gao et al., (2019) (Nat. Cell Biol. 21, 687-699).

1.3 Human Trophoblast Stem Cell Line (hTSCs) Establishment and STB Differentiation

To generate the hTSC cell lines, the single cell suspension of hEPSCs were plated on a 6-well plate (1-2×10³/well) pre-coated with 10 mg/mL Geltrex™ LDEV-Free Reduced Growth Factor Basement Membrane Matrix or Matrigel and cultured in human trophoblast stem cell medium (hTSCM) for 7-10 days. Then single colonies of the differentiated cells on the 6-well plate were picked and expanded. The expanded cell lines were validated with trophoblast markers and RNA-Seq analysis. The established TSC cell lines were maintained in the hTSCM medium and passaged at ratio 1:4-1:5 every 3-5 days. The hTSCM was DMEM/F12 based medium supported with: 110 μM β-mercaptoethanol, 0.2% FBS, 0.5% Penicillin-Streptomycin-Glutamine, 0.3% BSA, 1×ITS-X supplement, 50.0 μg/mL Vc, 50.0 ng/ml EGF, 2.0 μM CHIR99021, 0.5 μM A83-01, 1.0 μM SB431542, 0.8 μM VPA, and 5.0 μM Y27632. Established TSC cells were seeded at 1.0×10⁵-2×10⁵ cells per well in a six-well plate pre-coated with 10 mg/mL Geltrex™ LDEV-Free Reduced Growth Factor Basement Membrane Matrix or Matrigel, and were induced to differentiate into STBs after culturing in syncytiotrophoblasts medium (STBM) for around 6 days. The STBM comprises: DMEM/F12 supplemented with 110 μM β-mercaptoethanol, 0.5% Penicillin-Streptomycin, 0.3% BSA, 1×ITS-X, 2.5 μM Y27632, 2 μM Forskolin, and 4% KnockOut Serum Replacement. The cells became early syncytiotrophoblasts on day 2-3, and mature syncytiotrophoblasts around day 6.

### 1.4 Protocols for Mechanism Study During TSC-STB Process

ELISA: On day 3 and/or day 6 of STB differentiation, the culture medium of each well was collected, and the supernatant was obtained by centrifugation. Sterile STBM was used as blank control. ELISA was carried out according to manufacturer specification (Human-hCG-ELISA-Kit-protocol-book-v4-ab100533 (website).pdf (abcam.com)) to measure secreted β-hCG. In particular, 100 μl TMB was added to the supernatant and blank control for incubation for 30 min, after which stop solution was added and measurements were taken using a spectrophotometer at 450 nM.

RT-qPCR: Total RNA was extracted using RNeasy Mini Kit (Qiagen) per manufacturer specification. Extracted RNA was then reverse transcribed to cDNA using Fastking gDNA diselling RT SuperMix (Tiangen). Gene expression was measured using PowerUp™ SYBR™ Green Master Mix (Applied Biosystems) and StepOnePlus™ Real-Time PCR (Applied Biosystems) with the primers listed in Table E1A. Raw gene expression data was normalized to GAPDH by using the ΔCt method. Statistical analysis was carried out using either one/two-tailed student's t-test in Prism 8 (GraphPad).

TABLE E1A
Primers used in RT-qPCR (5′ to 3′)

| Gene | | SEQ | | SEQ |
|---|---|---|---|---|
| Name | Forward Primer | ID NO | Reverse Primer | ID NO |
| NANOG | TGAACCTCAGCTACAAAC | 3 | TGGTGGTAGGAAGAGTAAAG | 25 |
| | AG | | | |
| OCT4 | CCTCACTTCACTGCACTGT | 4 | CAGGTTTTCTTTCCCTAGCT | 26 |
| | A | | | |
| SOX2 | TTCACATGTCCCAGCACTA | 5 | TCACATGTGTGAGAGGGGCAG | 27 |
| | CCAGA | | TGTGC | |
| CDX2 | TTCACTACAGTCGCTACAT | 6 | TTGATTTTCCTCTCCTTTGCTC | 28 |
| | CACC | | | |
| GATA3 | ACATCTCGCCCTTCAGCCA | 7 | CATGGCGGTGACCATGCTGGA | 29 |
| | C | | | |
| KRT7 | AGGATGTGGATGCTGCCT | 8 | CACCACAGATGTGTCGGAGA | 30 |
| | AC | | | |
| TEAD4 | CAGGTGGTGGAGAAAGTT | 9 | GTGCTTGAGCTTGTGGATGAA | 31 |
| | GAGA | | G | |
| TFAP2C | ACAGGATCCATGTTGTGG | 10 | ATACTCGAGTTTCCTGTGTTTC | 32 |
| | AAAATAACCGAT | | TCCATTTT | |
| TP63 | AGAAACGAAGATCCCCAG | 11 | CTGTTGCTGTTGCCTGTACGTT | 33 |
| | ATGA | | | |
| CGA | TCCATTCCGCTCCTGATGT | 12 | CGTCTTCTTGGACCTTAGTGG | 34 |
| | GCA | | AG | |
| CGB | ACCCTGGCTGTGGAGAAG | 13 | ATGGACTCGAAGCGCACA | 35 |
| | G | | | |
| ERVW1 | GTTAATGACATCAAAGGC | 14 | CCCCATCTCAACAGGAAAACC | 36 |
| | ACCC | | | |
| SDC1 | GCTGACCTTCACACTCCCC | 15 | CAAAGGTGAAGTCCTGCTCCC | 37 |
| | A | | | |
| HLA-G | CAGATACCTGGAGAACGG | 16 | CAGTATGATCTCCGCAGGGT | 38 |
| | GA | | | |
| MMP2 | TGGCACCCATTTACACCTA | 17 | ATGTCAGGAGAGGCCCCATAG | 39 |
| | CAC | | A | |
| ITGB6 | CTCAACACAATAAAGGAG | 18 | AAAGGGGATACAGGTTTTTCC | 40 |
| | CTGGG | | AC | |
| GABRP | TTTCTCAGGCCCAATTTTG | 19 | GCTGTCGGAGGTATATGGTGG | 41 |
| | GT | | | |
| MUC16 | GGAGCACACGCTAGTTCA | 20 | GGTCTCTATTGAGGGGAAGGT | 42 |
| | GAA | | | |
| IL6 | GTCAGGGGTGGTTATTGC | 21 | AGTGAGGAACAAGCCAGAGC | 43 |
| | AT | | | |
| IL28A | TCCAGTCACGGTCAGCA | 22 | CAGCCTCAGAGTGTTTCTTCT | 44 |
| TNF | CTCTTCTGCCTGCTGCACT | 23 | ATGGGCTACAGGCTTGTCACT | 45 |
| | TTG | | C | |
| GAPDH | CAAATTCCATGGCACCGTC | 24 | ATCGCCCCACTTGATTTTGG | 46 |
| | A | | | |

Immunofluorescence staining (IF): Samples were fixed in 4% paraformaldehyde (Sigma Cat. P6148) at room temperature for 15 min, permeabilized with 0.3% Triton X-100 (Sigma. Cat. T8787) for 10 min, and blocked for 0.5-1 h with 5% donkey serum (Sigma. Cat. D9663) as well as 1% BSA (Sigma. Cat. A2153) in PBS. This was followed by incubation with primary antibodies in a 4° C. cold room overnight. After aspirating the primary antibodies and washing in PBS for three times (10 min/time), the cells were incubated with fluorophore-conjugated secondary antibodies at room temperature for 1 h. After another 3 rounds of washing in PBS, the cells were counterstained with 10 g/mL DAPI (Thermo Fisher Scientific. Cat. 62248) for 10 min to mark nuclei and were imaged under a confocal microscope.

Western blot: The proteins were extracted with RIPA lysis (thermofisher, Cat. 89901) and Pierce Protease Inhibitor Mini Tablets (thermofisher, Cat. A32953). Protein concentrations were measured with Pierce™ BCA Protein Assay Kits (thermofisher, Cat. 23227). Then samples with certain amount of NuPAGE™ LDS Sample Buffer (4×) (thermofisher, Cat. NP0008) were boiled at 70 degree for 10 min. Boiled samples were loaded with the same amount and separated in 12% polyacrylamide gels (Thermofisher Cat. NP0343BOX) and transferred to PVDF membranes using the Bio-Rad transblot turbo system according to the manufacturer's guidance. Images were developed by the ChemiDoc Imaging System and processed with ImageJ 1.47v. The following primary antibodies were used: anti-GAPDH (thermosfisher, Cat. AM4300, 1:10000 for western blot), anti-Ki67 (thermofisher, Cat. MA5-14520, 1:1000 for western blot, 1:200 for IF), anti-HERVK-gag (Austral Biologicals, Cat. HERM-1811-5, 1:200 for IF), anti-TRF1 (Abcam, Cat. ab 10579, 1:100 for IF), anti-ZO-1 (CST, Cat. 13663, 1:200 for IF), anti-CGB3 (Abcam, Cat. Ab131170, 1:200 for IF), anti-H3 (CST, Cat. 4499 1:2000 for western blot), anti-H3K9mc3 (Abcam, Cat. ab8898, 1:1000 for western blot), anti-E-cadherin (CST, Cat. 14472, 1:200 for IF), anti-p38 MAPK (thermofisher, Cat. 338700, 1:1000 for western blot), anti-LaminB1 (thermofisher, Cat. 702972, 1:1000 for western blot, 1:200 for IF), anti-p27 Kip1 (thermofisher, Cat. PA527188, 1:1000 for western blot), anti-Hp1 gamma (thermofisher, Cat. MA3054, 1:1000 for western blot, 1:300 for IF), anti-SIRT1 (CST, Cat. 8469S, 1:1000 for western blot), anti-SIRT6 (CST, Cat. 12486S, 1:1000 for western blot), anti-SIRT3 (thermofisher, Cat. MA514910, 1:1000 for western blot), anti-p53 (thermofisher, Cat. MA512557, 1:1000 for western blot, 1:200 for IF), anti-gammaH2AX (CST, Cat. 2577s, 1:1000 for western blot, 1:200 for IF), anti-IL-6 (thermofisher, Cat. MA523698, 1:50 for IF), anti-hCG (thermofisher, Cat. 14-6508-82, 1:1000 for western blot, 1:200 for IF). Secondary antibodies used: Goat anti Mouse IgG (H+L) HRP (thermofisher, Cat. 31430, 1:2000 for western blot), Goat anti-Rabbit IgG (H+L) HRP (thermo fisher, Cat. 31460, 1:2000 for western blot), Goat anti-Rabbit IgG (H+L) Alexa Fluor 647 (thermofisher, Cat. A21244, 1:500 for IF), Rabbit anti Mouse IgG (H+L) Alexa Fluor 647 (thermofisher, Cat. A21239, 1:500 for IF), Donkey anti Rabbit IgG (H+L) Alexa Fluor 488 (thermofisher, Cat. A21206, 1:500 for IF), Donkey anti Mouse IgG (H+L) Alexa Fluor 488 (thermofisher, Cat. A21202, 1:500 for IF), Donkey anti Mouse IgG (H+L) Alexa Fluor 594 (thermofisher, Cat. A21203, 1:500 for IF), Donkey anti Rabbit IgG (H+L) Alexa Fluor 594 (thermofisher, Cat. A21207, 1:500 for IF).

Flow cytometry: Cells were digested with 0.25% trypsin/EDTA for 2-3 min at 37° C. and dissociated to single cells by pipetting. The dissociated cells were filtered through a 40-mm nylon mesh (Kangning cat. 352235) to remove cell clumps. After centrifugation, the cells were fixed using Fixation Medium (BD Cytofix, Cat. 554655) according to the manufacturer's protocol and the washed cells were stored at 4° C. in PBS supplemented with 2% FBS (Gibco. Cat. 10270) before analysis with flow cytometry. All the samples were assayed by ACEA NovoCyte Quantcon. 488 nm (530/30 bandpass filter) and 561 nm (610/20 bandpass filter) channels were used to detect FITC and exclude autofluorescence. 405 nm (445/45 bandpass filter) channel was used to detect DAPI positive cells. FACS data was analyzed by the Flowjo software.

RNA sequencing: RNA sequencing was carried out at various time points. Adapter sequences and low-quality 3′ end sequences were removed using Cutadapt. Processed reads were mapped to the human hg38 genome assembly by hisat2. Gene annotation form Ensembl was used. FeatureCounts was used to quantify gene expression. Genes with mean count number <5 were filtered out. Transposable element annotations were obtained from UCSC Genome Browser (RepeatMasker). SQUIRE with “total” mode was used to quantify TE expression. DEScq2 was used to analyze differentially expressed genes and TEs. R package clusterprofiler was used for gene ontology (GO) and KEGG analysis. GSEA (Gene Set Enrichment Analysis) was performed by GSEApy, and the gene sets were downloaded directly from https://www.gsca-msigdb.org. Bigwig files for RNA-seq signal were generated by bamCoverage from Deeptools and IGV was used for visualization. For the RNA-seq signal from endogenous retroviruses HERV-W, HERV-FRD, and HERV-K, GenBank: AY101582.1, BC068585.1, and N675077.1 were used and the reads were mapped by hisat2 and quantified. For data deposited in E-MTAB-10429, the processed count table was used directly. The difference in expression in TSC and STB (on day 2, day 4, day 6, or day 8) was calculated as log 2 fold change for each gene (with adjusted p-value <0.01). The top 100 up-regulated and top 100 down-regulated genes in TSC and STB were selected after ranking the genes by absolute log 2 fold change.

### 1.5 Agent Screening Protocol and Cell Viability Validation

Same number of TSCs in robust growing state were seeded in STBM with/without a candidate agent in a black Polystyrene TC-treated 96-well Microplate pre-coated with 10 mg/ml Geltrex™ LDEV-Free Reduced Growth Factor Basement Membrane Matrix or Matrigel (1.5×10⁴-2×10⁴ cells per well). On each screening microplate, 3 wells of control (cells only with 0.5% DMSO in 100 μL STB growth medium) and 3 wells of positive control (some well-known anti-ageing agents in certain concentration) were included. The cells were cultured at 37° C. and 5% CO₂ with saturating humidity and differentiated into mature STB in the medium for 6 days. Cell morphology was monitored daily under a microscope. The signal readouts of reporter molecules was assessed on day 2, day 4, and day 6. On day 6, cells in each well were fixed and stained with DAPI or Hoechst, and cell count and intensity (both reporter and nuclei) were assessed using high content microscopy to assess the agent's effects and cell viability. The agents, especially those that affect cell viability will also be tested at different concentrations and at different day during the STB differentiation process.

### 1.6 Reporter Cell Lines

Based on template plasmids pCD31-5HA-T2A-H2B-EGFP-loxp-EF1a-puro-loxp-3HA (SEQ ID NO: 1) and hSpCas9-2A-Puro (PX459) (addgene #62988; SEQ ID NO: 2), plasmids for CGA, CGB3, and ERVW1 were constructed for reporter cell lines. The full sequences for human CGA, CGB3, and ERVW1 were obtained from Ensembl genome browser 110 (https://asia.ensembl.org/index.html). The gRNA of each gene was at 3′ of the stop codon (less than 100 bp away from stop codon) and inserted into the U6-gRNA-Cas9 construct by digestion and T4 ligation. Homologous arms (5HA and 3HA parts of each gene) (1000-1500 bp) were inserted 5′ of the stop codon and 3′ of the gRNA targeted site, respectively. Using homologous recombination (CloneExpress II One Step Cloning Kit), 5HA and 3HA were inserted into the pCD31-5HA-T2A-H2B-EGFP-loxp-EF1a-puro-loxp-3HA to get the CGA-T2A-H2B-GFP, CGB3-T2A-H2B-GFP and ERVW1-T2A-H2B-GFP reporter constructs. The reporter plasmid of each gene together and their corresponding gRNA construct were transfected into EPSCs. With puromycin selection for 7 days, survived EPSC colonies were confirmed through genotyping for the reporter insertion in the genome. The confirmed EPSC colonies were then expanded and induced to differentiate to TSC cell lines for further studies.

### Example 2: STB Differentiation from EPSC-Derived TSC

This example shows that M1-EPSCs can be efficiently induced to differentiate into TSCs, and the TSCs can differentiate into STBs that express the relevant markers.

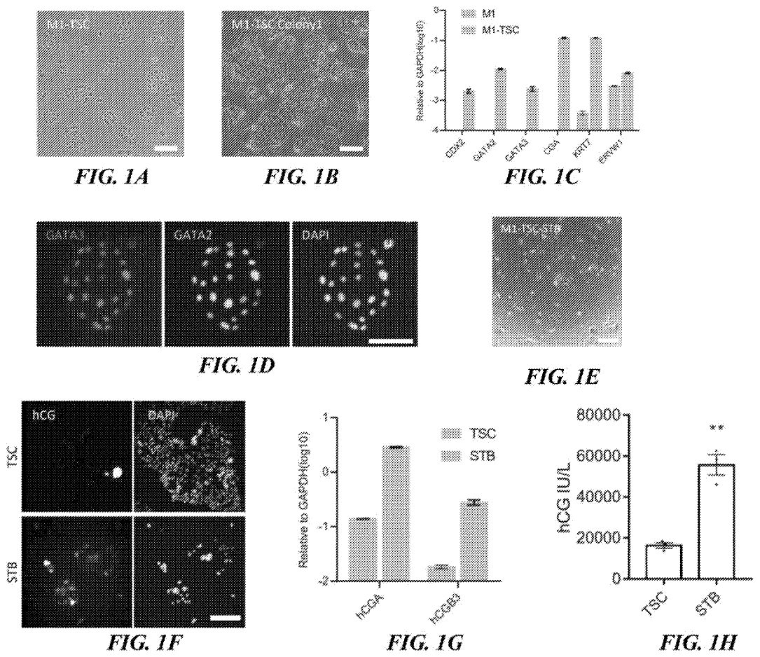

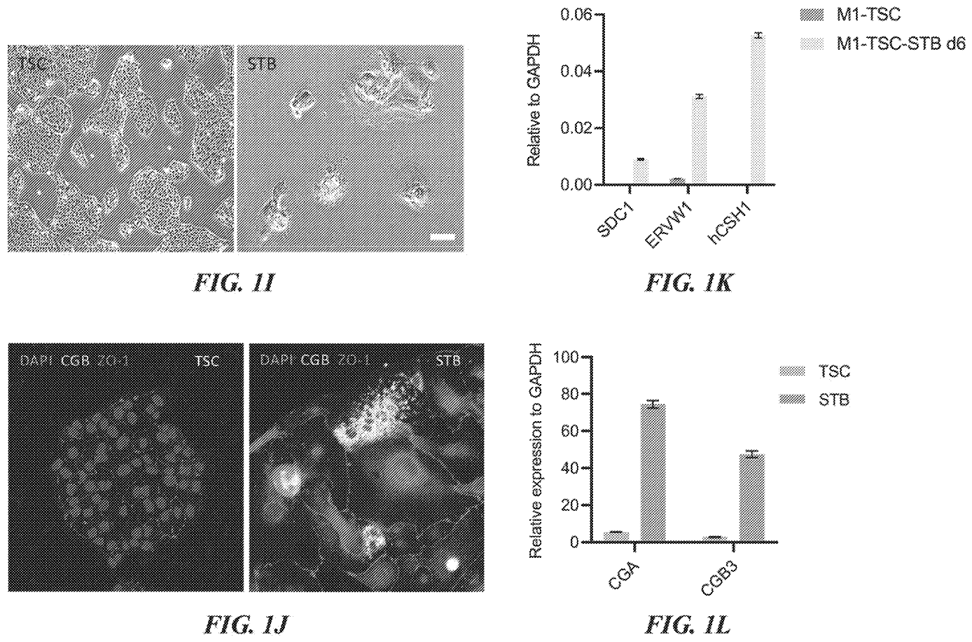

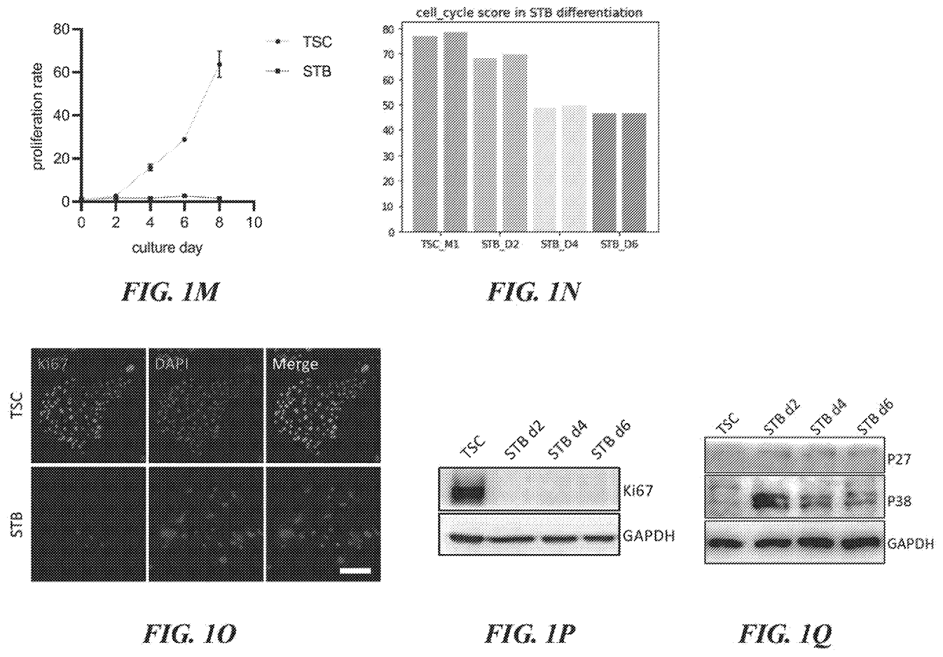

As shown in FIG. 1A, M1-EPSCs cultured in human trophoblast stem cell medium as described in Example 1 were induced to differentiate into multiple TSC single cell clones. The morphology of the established cell lines after single-clonal M1-TSC expansion was stable (FIG. 1B). Differentiation markers (CDX2, GATA2, GATA3, CGA, KRT7, and ERVW1) were expressed at significantly higher levels in TSCs compared to M1-EPSCs (FIG. 1C). Immunofluorescence also shows that GATA2 and GATA3 were highly expressed in TSCs (FIG. 1D).

M1-TSCs were then induced to differentiate into STBs in STBM as described in Example 1. Differentiated cells showed the expected morphology for STBs (FIGS. 1E, 1I, and 1J). hCG was expressed in STBs at a higher level than in TSCs, as shown by immunofluorescence (FIG. 1F) and ELISA (FIG. 1H). CGA and CGB3 levels were higher in STBs than in TSCs, as indicated by RT-qPCR and immunofluorescence (FIGS. 1G, 1J, and 1L). STB markers SDC1, ERVW1, and CSH1 were expressed significantly higher in STBs on day 6 (FIG. 1K). In addition, the STBs were in a proliferation arrest state as demonstrated by the low proliferation rate (FIG. 1M), low cell cycle scores from the RNA sequencing data (FIG. 1N), low expression of the proliferation marker Ki67 (FIGS. 1O and 1P), and increased expression of cell cycle inhibitors p27 and p38 (FIG. 1Q).

### Example 3: Aging-Related Characteristics During TSC-STB Differentiation

This example shows that the various markers related to aging show changing patterns during TSC-STB differentiation (RNA-seq data) that mimic the normal aging process in human (Table E2A).

TABLE E2A
Aging-related characteristics

| | | Change in | Change in |
|---|---|---|---|
| Aging | Aging | normal aging | TSC-STB |
| Hallmark | Mechanism | process | differentiation |
| DNA repair | NER | Decrease | Decrease |
| | BER | Decrease | Decrease |
| | MMR | Decrease | Decrease |
| | Fanconi anemia pathway | Decrease | Decrease |
| | HR | Decrease | Decrease |
| | NHEJ | Decrease | Decrease |
| Epigenetic | Linker histone | Increase | Increase |
| alteration | | | |
| Cell cycle | Cell cycle | Decrease | Decrease |
| Faulty | Insulin | Decrease | Decrease |
| nutrient | Insulin resistance | Increase | Increase |
| sensing | AMPK | Decrease | Decrease |
| | SIRT family | Decrease | Increase * |
| Mitophagy | Mitophagy | Increase | Increase |
| Chronic | Pro-inflammation | Increase | Increase |
| inflammation | cytokines | | |
| Senescence | Senescence pathways | Increase | Increase |
| | Senescence inhibitors | Decrease | Decrease |
| | Epigenetics of senescence | Increase | Increase |

* SIRT family members increased in STB cells, at RNA level, which was not consistent with normal ageing, but western blot showed they decreased in STB cells comparing to TSC cells, which was consistent with normal ageing.

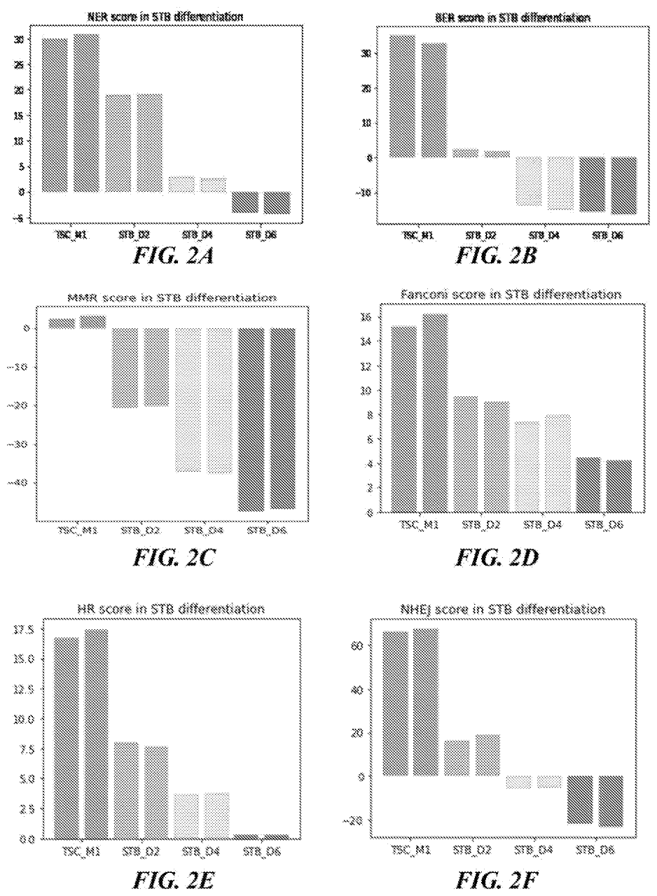

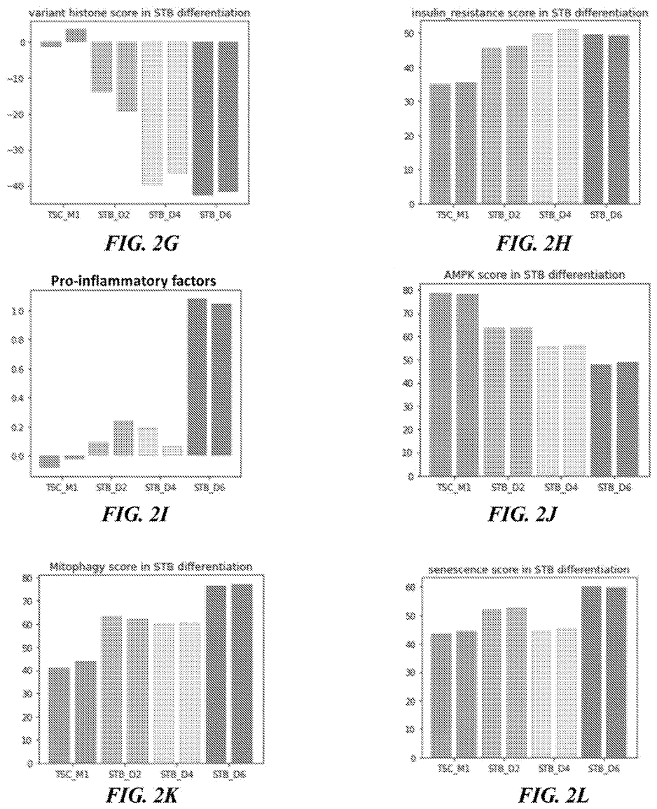

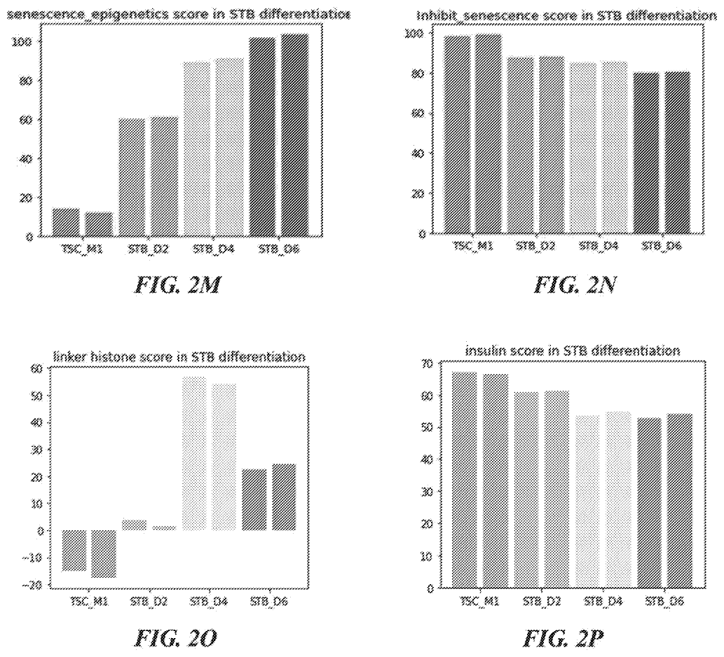

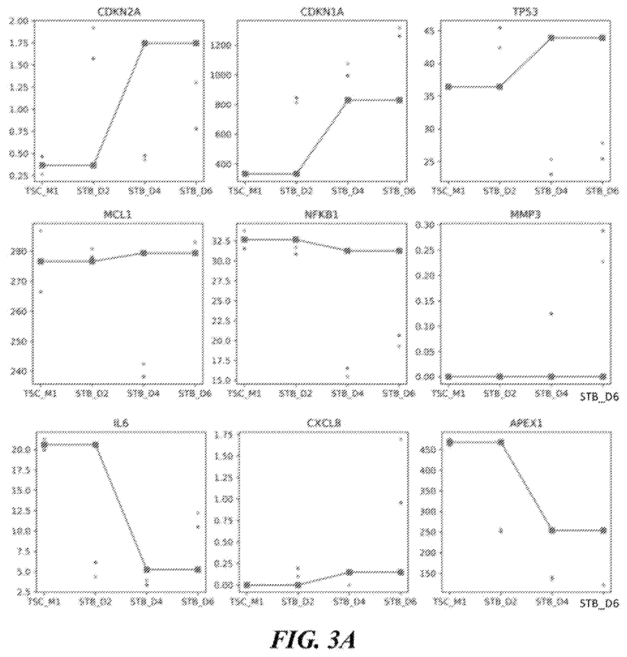

The level of NER gradually decreased from TSC to STB, suggesting that with the maturation and aging of STB, the repair ability gradually decreased (FIG. 2A). BER also decreased during the process of differentiation and maturation from TSC to STB, suggesting a decline in repair ability (FIG. 2B). MMR score also decreased as TSCs differentiate to STBs, suggesting aging (FIG. 2C). As shown in FIG. 2D, the Fanconi anemia signaling pathway gradually decreased with the differentiation from TSC to STB, suggesting a decline in repair capacity. As shown in FIG. 2E, homologous recombination decreased as with the differentiation from TSC to STB, suggesting aging. As shown in FIG. 2F, nonhomologous end joining (NHEJ) decreased with the differentiation from TSC to STB, suggesting aging.

As shown in FIG. 2G, Variant histone score decreased with the differentiation from TSC to STB, suggesting aging.

Another indicator of cellular aging, insulin resistance in insulin metabolism increased with the differentiation from TSC to STB (FIG. 2H), while insulin score increased (FIG. 2P).

The expression of pro-inflammatory factors gradually increased with the differentiation from TSC to STB, and reached the highest level day 6, suggesting that aging-induced pro-inflammatory factors increased (FIG. 2I).

The mTOR/AMPK pathway can sense the energy demand of the microenvironment and regulate the cellular metabolic pathway. It can be seen that with the differentiation and aging from TSC to STB, the mTOR/AMPK score decreased, indicating that the metabolic capacity and level of the cells are reduced (FIG. 2J).

Mitophagy is the selective removal of damaged mitochondria by autophagosomes, followed by their catabolism by lysosomes. As shown in FIG. 2K, mitophagy gradually increased with STB differentiation, suggesting increased senescent organelles.

Both overall senescence score and senescence related epigenetic score (including linker histone score) increased, while the score related to senescence inhibition decreased (FIGS. 2L-20).

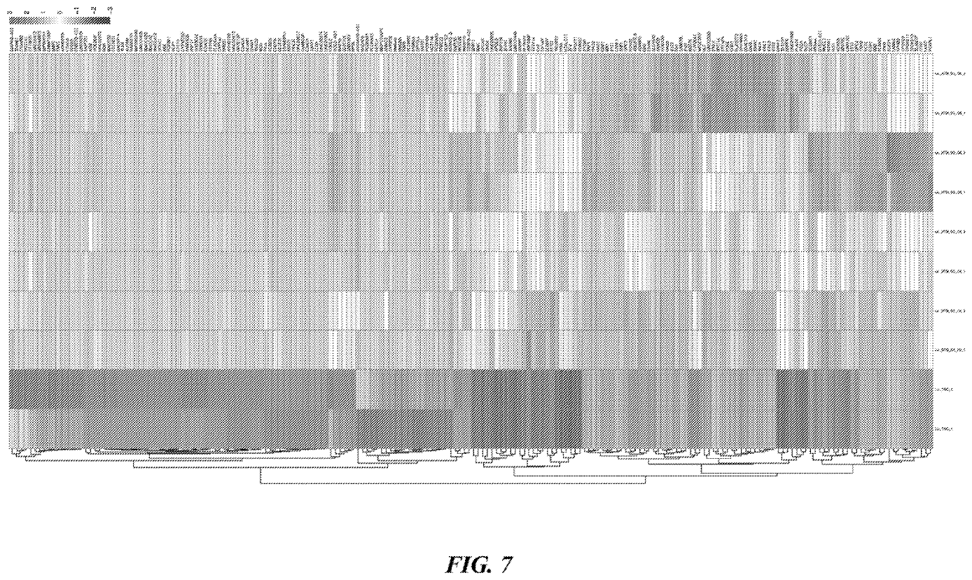

The cells were further tested for senescence-associated secretory phenotype (SASP) and aging-related proteins. TSCs were derived from M1-EPSCs, and induced to differentiate into STBs. During the differentiation into STBs, cells were collected for transcriptome sequencing on day 2, day 4, and day 6. The sequencing data was analyzed as described in Example 1. RT-qPCR was carried out at the beginning and end of differentiation as described in Example 1. Differentially expressed genes are shown in FIG. 7. The top 100 upregulated and downregulated genes at STB day 2 vs. TSC, STB day 4 vs. TSC, STB day 6 vs. TSC, and STB day 8 vs. TSC are shown in Table E2B.

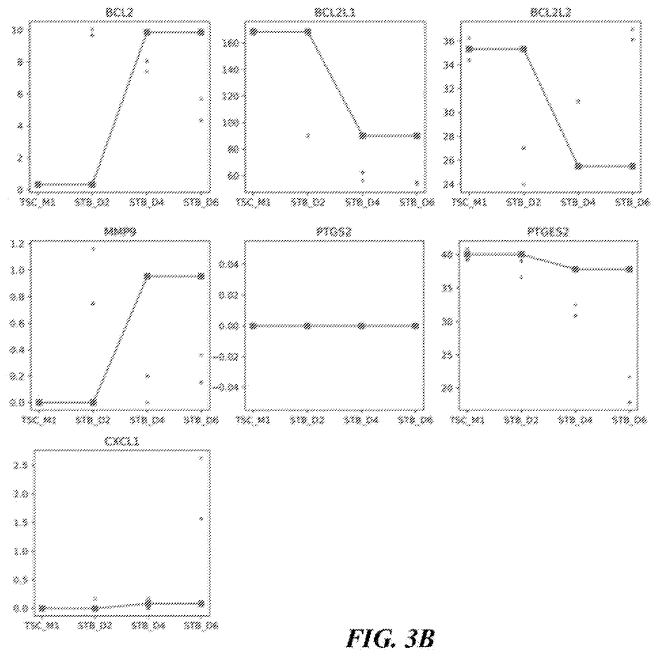

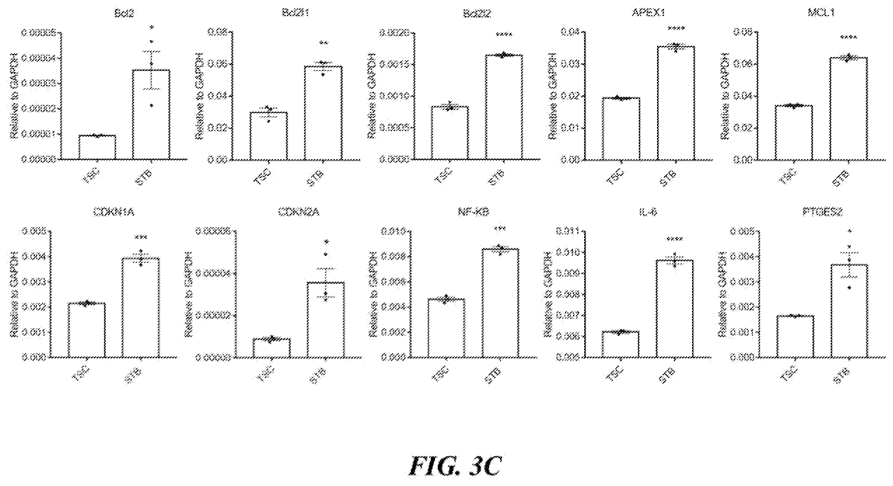

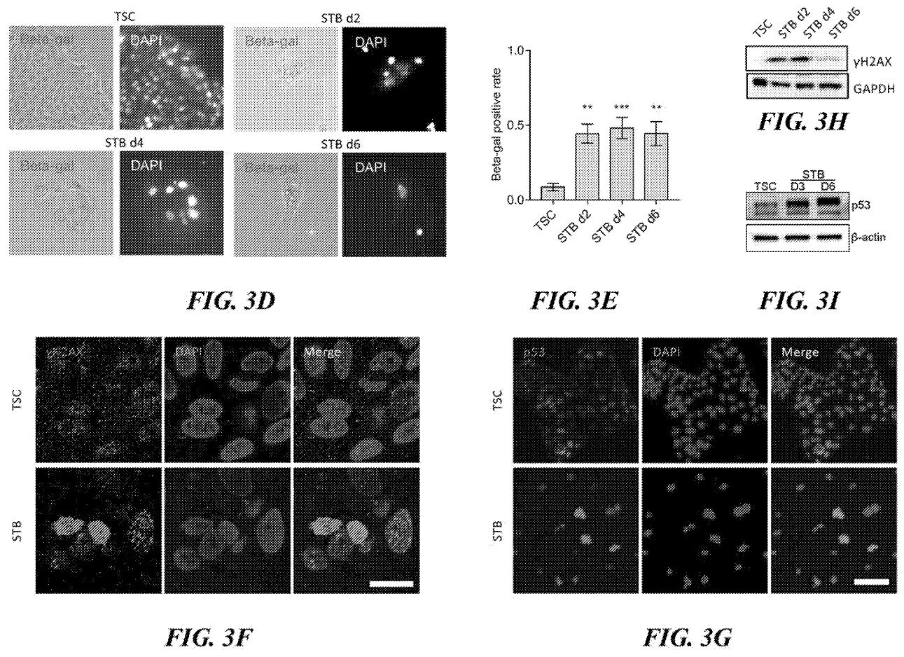

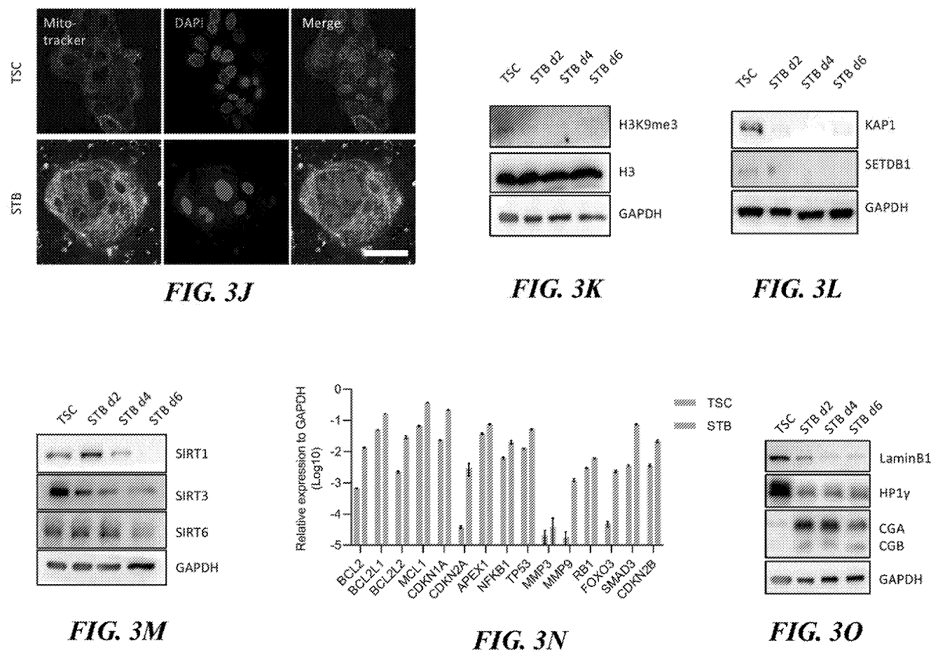

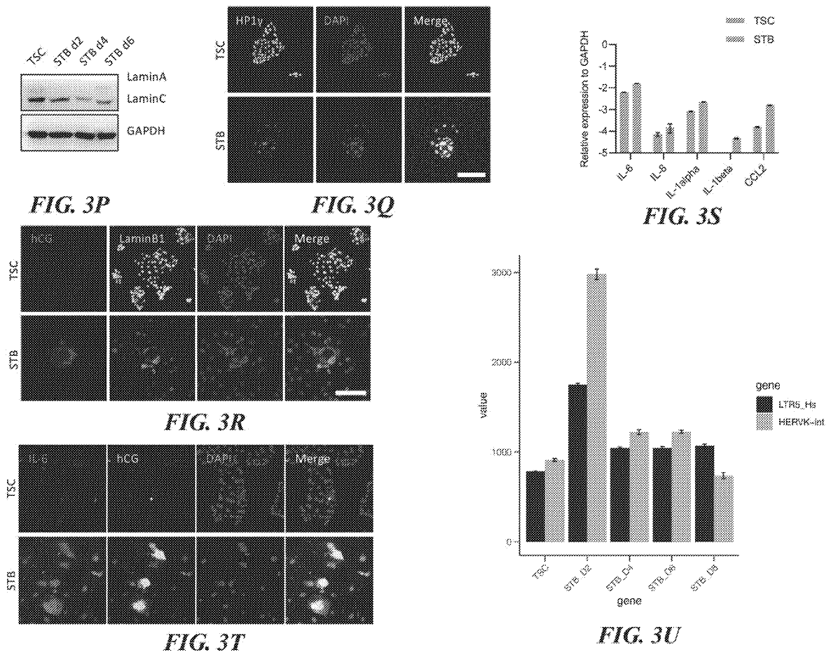

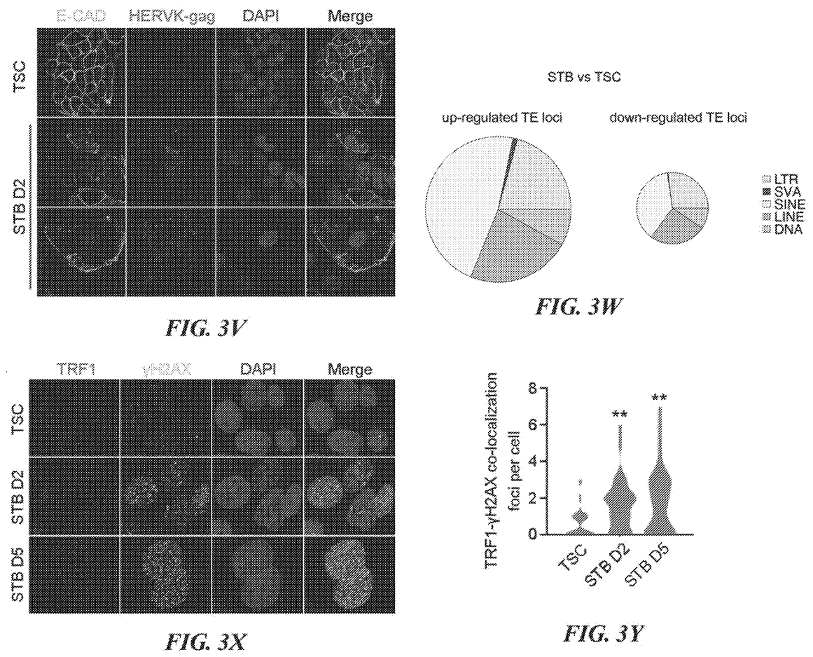

As shown in FIGS. 3A-3C, 3N, and 3S, senescence-associated secretory phenotype (SASP) and aging-related proteins showed different expression levels in TSCs and STBs. In particular, BCL2, MCL1, CDKN1A, CDKN2A, APEX1, NFKB1, TP53, MMP3, MMP9, RB1, FOXO3, SMAD3 and CDKN2B are significantly higher expressed in STB cells than TSC cells. IL-6, IL-8, IL-1α, IL-1β and CCL2 also had higher expression in STBs than in TSCs (FIGS. 3S-3T). Beta-galactosidase, γH2AX and p53 expression also increased as TSCs differentiated towards STBs (FIGS. 3D-3I). STBs were also shown to have increased mitochondria mass as shown by immunostaining of Mito-tracker (FIG. 3J). Other biomarkers, such as H3K9me3, SETDB1/KAP1 complex, HP1γ, nuclear lamin proteins (LaminA, LaminB1, LaminC) showed decreased expression as TSCs differentiate towards STBs (FIGS. 3K, 3O-3R) which indicated that the genomic instability disorder in STB cells increased compared to TSC cells. Classical sirtuin family members (SIRT1, SIRT3, SIRT6) which relate closely to longevity decreased in STB cells (FIG. 3M) even though they increased at the RNA level (Table E2B). In addition to increasing or decreasing trends, some biomarkers (such as LTR5 and HERVK) had a more complicated pattern where expression was increased on day 2 during TSC-STB differentiation and decreased thereafter (FIGS. 3U-3V). Pie charts were generated to show the classes of up-regulated and down-regulated transposable elements (TE) loci after TSC differentiation. The size of each pie chart area is proportional to the number of TE loci (FIG. 3W). One of telomere attrition related molecule TRF1 increased during STB differentiation, and there was more colocalization foci with gamma H2AX in STB than in TSC cells (FIGS. 3X-3Y).

TABLE E2B
Top 100 upregulated and downregulated genes.

| STB_D2_vs_{—} | STB_D2_vs_{—} | STB_D4_vs_{—} | STB_D4_vs_{—} | STB_D6_vs_{—} | STB_D6_vs_{—} | STB_D8_vs_{—} | STB_D8_vs_{—} |
|---|---|---|---|---|---|---|---|
| TSC_up | TSC_down | TSC_up | TSC_down | TSC_up | TSC_down | TSC_up | TSC_down |
| PEG3 | MAGEA2B | DDX3Y | MAGEA12 | OAS2 | MAGEA2B | OAS2 | MAGEA2B |
| DDX3Y | MAGEA2 | PEG3 | CSAG1 | PEG3 | MAGEA2 | OAS3 | MAGEA2 |
| RPS4Y1 | MAGEA12 | RPS4Y1 | MAGEA2B | DDX3Y | MAGEA12 | PEG3 | MAGEA12 |
| TXLNGY | CSAG1 | PLAC4 | MAGEA2 | OAS3 | TSSC2 | DDX3Y | GALNT14 |
| KDM5D | MAGEB2 | TXLNGY | MAGEB2 | CSH2 | CSAG1 | LGALS14 | CSAG1 |
| USP9Y | SH3BGRL | KDM5D | SH3BGRL | RPS4Y1 | MAGEB2 | RPS4Y1 | MAGEB2 |
| THEM6 | CSAG3 | CSH2 | LINC02413 | GH2 | CSAG3 | PLAC4 | LINC01405 |
| CAT | LINC02413 | LGALS14 | TSSC2 | CSH1 | LINC02413 | CSH2 | CDX1 |
| ZFY | TSSC2 | OAS3 | CDX1 | LAIR2 | HMGA2 | STEAP4 | PTCHD1 |
| LYNX1 | PTCHD1 | OAS2 | ARHGEF9 | PLAC4 | ARHGEF9 | IFNL3 | ARHGEF9 |
| EIF1AY | HMGA2 | ZFY | CHRM3 | LGALS14 | FAM86GP | GH2 | AJAP1 |
| PLAC4 | ARHGEF9 | GH2 | GALNT14 | KDM5D | CSAG2 | IFNL2 | LINC02413 |
| LAIR2 | GALNT14 | WIPF1 | AJAP1 | TXLNGY | AJAP1 | CSH1 | TLR4 |
| WIPF1 | FAM86GP | HLF | CSAG3 | THEM6 | GALNT14 | KDM5D | DDX11L2 |
| APOC3 | CSAG2 | LAIR2 | ALCAM | WIPF1 | OR7E12P | HLF | OR7E12P |
| LY6K | AJAP1 | USP9Y | TLR4 | LYNX1 | PTCHD1 | USP9Y | LINC02474 |
| ZNF558 | LPAR1 | STEAP4 | TGFA | XAF1 | LINC02474 | LYNX1 | DCLK1 |
| OAS3 | TLR4 | THEM6 | DDX11L2 | IFNL3 | DCLK1 | LAIR2 | LINC02575 |
| LINC02055 | DDX11L2 | CAT | OR7E12P | ZFY | PRKCQ-AS1 | TXLNGY | PRKCQ-AS1 |
| LGALS14 | OR7E12P | LYNX1 | PTCHD1 | LAMA4 | PINCR | THEM6 | CERS6 |
| ZNF826P | CDCA7L | LY6K | CDCA7L | APOC3 | SNHG14 | ZFY | CHRM3 |
| TTTY14 | LINC01405 | APOC3 | HMGA2 | ADAM12 | FAM162B | XAF1 | NDN |
| NLGN4Y | PI15 | EIF1AY | PI15 | USP9Y | PRKCQ | IFIT2 | PRKCQ |
| TMSB4Y | LINC02474 | ADAM12 | MYEOV | STEAP4 | CYP4F26P | ADAM12 | CYP4F26P |
| SEC14L4 | DCLK1 | CSH1 | LINC02474 | HLF | MSN | WIPF1 | TSSC2 |
| LINC01446 | SNHG14 | ZNF558 | PRKCQ-AS1 | IFNL2 | PCDHA10 | APOC3 | PCDHA10 |
| ZFP41 | NDN | IFNL3 | LINC02617 | SPDYE13 | EML5 | CAT | PIMREG |
| LGALS13 | PRKCQ | LGALS13 | PINCR | ZNF558 | LINC00379 | CNN1 | IMPDH1P4 |
| ZNF257 | PINCR | SCUBE1 | HMGN5 | CAT | PCDHA12 | ZNF558 | LINC00379 |
| STEAP4 | CDX1 | SPDYE13 | FAM162B | LY6K | IMPDH1P4 | EIF1AY | RPE65 |
| GH2 | IMPDH1P4 | WNT9B | NDN | EIF1AY | CDX1 | PDK4 | ZNF57 |
| MAG | RPE65 | FLT4 | EYA1 | CD22 | RPE65 | IFIT3 | SH3BGRL |
| LINC00278 | UCP1 | LINC02055 | PRKCQ | IFIT2 | ZNF57 | IFNL1 | PINCR |
| TPTEP1 | MSN | CD22 | LINC01405 | IFIT3 | CHMP1B2P | MYO1A | SNTB1 |
| CD22 | CHMP1B2P | TTTY14 | PCDHA10 | OAS1 | TLR4 | SAMD9L | UGT1A6 |
| AIRE | SNTB1 | ZFP41 | PCDHA12 | SCUBE1 | PKIA | LY6K | BMP5 |
| CNN1 | BMP5 | IFIT2 | RPE65 | CSHL1 | DSG2-AS1 | SPANXN5 | C10orf82 |
| APOA5 | DPYSL4 | MMEL1 | ZNF57 | GPC3 | UGT1A6 | GPC3 | DPYSL4 |
| ZFY-AS1 | FBXW12 | XAF1 | CLDN10 | LINC02055 | DDX11L2 | CSHL1 | STAMBPL1 |
| OAS2 | STAMBPL1 | TMSB4Y | SFTA1P | WNT9B | LINC01405 | OASL | E2F6P4 |
| ANOS2P | A2M | CMKLR2 | CHMP1B2P | CNN1 | BMP5 | OAS1 | PI15 |
| GNG4 | KCNJ5 | LINC01446 | SNTB1 | FLT4 | C10orf82 | SPDYE13 | PNPO |
| CMKLR2 | E2F6P4 | KCNS3 | C10orf82 | BST2 | DPYSL4 | FLJ16779 | NETO1 |
| OXGR1 | ALOX12P2 | GPC3 | DPYSL4 | SAMD9L | STAMBPL1 | TAC3 | LRRTM4 |
| HOXB7 | NETO1 | IFNL2 | MSN | ZFP41 | WHAMMP2 | PAPPA | KRT72 |
| RLN2 | LRRTM4 | LAMA4 | FBXW12 | GBP1 | HAPLN1 | ZFP41 | ATP8A1 |
| WNT9B | KRT72 | MAG | STAMBPL1 | CMKLR2 | PTPRB | WNT9B | PCDHB2 |
| LINC01949 | ATP8A1 | LINC00278 | E2F6P4 | MUCL1 | PLA2G4A | NLGN4Y | SNX18P7 |
| COX20P1 | PRKCQ-AS1 | ABCB1 | FAM83A | MX1 | NETO1 | IGHA1 | HMGN5 |
| SPANXN5 | PCDHB2 | LINC01949 | LINC01127 | IFNL1 | KRT72 | GBP1 | CSAG4 |
| NBPF6 | CSAG4 | MUCL1 | NETO1 | OASL | ST13P20 | TRIM40 | COL19A1 |
| PRKY | AMER1 | TPTEP1 | CSAG2 | LINC01446 | PCDHB2 | SPTA1 | PROM1 |
| IGHV3-74 | LINC01291 | SPANXN5 | KRT72 | SPANXN5 | SNX18P7 | MX1 | LINC01807 |
| CSH2 | LINC01807 | KCNJ2 | ATP8A1 | KCNJ2 | HMGN5 | LINC01949 | LINC02412 |
| UNC13A | LINC02412 | HTRA4 | SNX18P7 | ETV7 | MAP10 | FLT4 | MIR2052HG |
| BACE2 | MIR2052HG | ZNF826P | CSAG4 | NLGN4Y | CSAG4 | KCNJ2 | NCAM1 |
| TMC1 | NCAM1 | APOA5 | COL19A1 | KCNS3 | LINC02617 | LINC02055 | NECTIN1-DT |
| LINC01090 | PCDHA4 | AIRE | LINC01807 | IFITM1 | MIR3176 | TTTY14 | TEX35 |
| CYP26A1 | MYEOV | OASL | LINC02458 | MAG | UCP1 | LINC02533 | DMC1 |
| PSCA | CDH13 | SPTA1 | LINC02412 | TPTEP1 | LINC01807 | TPTEP1 | LINC02617 |
| PAEP | ALCAM | NLGN4Y | MIR2052HG | TTTY14 | NDN | NKAIN4 | FAM86GP |
| UTY | ARTN | PDK4 | NCAM1 | LGALS13 | LINC02412 | CMKLR2 | TERT |
| MIR4482 | PCDHA10 | PTPN22 | LPAR1 | MX2 | MIR2052HG | KRT17P2 | TRIM61 |
| ZNF835 | TRIM61 | GUCY2F | NECTIN1-DT | KCNK3 | NCAM1 | CCL22 | EML5 |
| RN7SL708P | TEX41 | PAEP | SLC7A11-AS1 | SEMA3B | CYP1A1 | LINC01446 | TEX41 |
| ZNF717 | PCDHA12 | PSCA | KCTD15 | PDK4 | SH3BGRL | ETV7 | PCDHA12 |
| WFDC21P | PTPRB | HTRA1 | LINC00379 | HTRA1 | CELF2 | SEMA3B | PCDHA11 |
| FLT4 | CYP2C8 | OXGR1 | ITGB1BP2 | APOA5 | ARMC3 | LAMA4 | CSAG3 |
| NOC2LP1 | PROM1 | ACVRL1 | PROM1 | FLJ16779 | UGT1A1 | MT-TH | TSPEAR-AS2 |
| NBPF4 | PCDHA11 | MAN1C1 | UGT1A1 | IGHA1 | ZSCAN16- | MMEL1 | RARRES1 |
| | | | | | AS1 | | |
| LY6D | FAM110C | ARNT2 | C1orf220 | TAC3 | NR0B1 | BST2 | LINC00668 |
| ZNF736P9Y | TGFA | H2BC8 | CA8 | ZNF826P | TRIM61 | LGALS13 | ZNF785 |
| OR2C3 | KRTAP2-3 | SEC14L4 | CDH13 | GNG4 | DIAPH2-AS1 | WFDC21P | MPV17L |
| FAM167A | ZNF57 | BGN | ZSCAN16- | HTRA4 | SAPCD1 | GUCY2F | EVX1 |
| | | | AS1 | | | | |
| GABRE | NACAP8 | UNC5A | RARRES1 | CYP26A1 | CYP2C8 | MUCL1 | FAM83A |
| GYG2P1 | IGSF1 | MIR100HG | TRIM61 | SHFL | PCDHA11 | CXCL10 | CHMP1B2P |
| ZNF630 | MPV17L | NBPF6 | ANKRD18B | GPC4 | MRPS18AP1 | FAM167A | ABHD12B |
| HLF | SIMC1 | IFIT3 | EML5 | TCAM1P | PCDHB5 | ACVRL1 | PKIA |
| KCNS3 | BUD13P1 | GBP1 | PRKG2 | WFDC21P | KRTAP2-3 | LINC00278 | C1RL-AS1 |
| MTNR1B | NRROS | ZFY-AS1 | PRPH2 | ITIH3 | NACAP8 | IFI27 | STK32B |
| NPR 1.00 | UGT1A6 | NPR 1.00 | FHL1 | KRT78 | CDKL2 | SAMD9 | LINC02582 |
| HOXC9 | STK32B | RLN2 | FAM86GP | SPDYE9 | FAM83A | IFITM1 | CTTNBP2 |
| LINC02701 | ROR2 | BACE2 | RBP5 | EPHB2 | DOC2GP | SCEL | TGFA |
| ZNF726 | HMGN5 | ZNF257 | UGT1A6 | ANGPTL4 | LPAR1 | ZNF826P | CTAGE3P |
| WSCD1 | LINC02617 | CGB2 | BMP5 | SPDYE11 | SNTB1 | CD22 | SNORD99 |
| KRT78 | LINC00379 | IGHV3-74 | STK32B | PSAPL1 | RARRES1 | AIRE | AMER1 |
| ETV7 | WDR90 | NBPF4 | CLEC20A | PSCA | RBP5 | NCF1C | LINC01291 |
| ZNF658 | PLA2G4A | LINC02701 | TEX35 | IFIT1 | LINC02575 | FN1 | ITPRIPL1 |
| GPC4 | CASC9 | IFNL1 | GLUD2 | IFI27 | DEPDC1 | MAN1C1 | FAM162B |
| NKAIN4 | CYP4F26P | GNG4 | CERS6 | MYO1A | MECOM | CYP26A1 | MCM10 |
| FLJ16779 | TEX35 | ZNF835 | SNHG14 | PAEP | KCNE3 | SHFL | AK4 |
| RERG | LINC02154 | CNN1 | KLHL4 | LINC01949 | ALCAM | DLGAP1 | MSN |
| BGN | GSTT2B | FAM167A | C1RL | LINC02533 | ABHD12B | SLC16A2 | KRT3 |
| DLX5 | IFFO1 | GPR143P | PTPRB | FGF21 | SSBP3-AS1 | KRT78 | CHST6 |
| HTRA4 | CELF2 | KRT78 | LINC00668 | ESAM | GLUD2 | PSCA | HHLA3-AS1 |
| SCUBE1 | CHRM3 | RN7SL708P | CHST6 | SLC16A2 | A2M | UNC13A | CSAG2 |
| LINC00323 | C1RL-AS1 | CGB1 | ITPRIPL1 | FN1 | CHST6 | APOA5 | NR0B1 |
| LY6E | RARRES1 | LY6D | GSTT2B | SAMD9 | HPDL | ANGPTL4 | EMB |
| NCF1C | RGPD2 | LINC02533 | TERT | NBPF6 | AK4 | NBPF6 | KCNE3 |
| GJA3 | HOMER2 | TMEM225B | DCLK1 | GTF2I-AS1 | FGFBP1 | CPE | UCP1 |

### Example 4: Effects of Anti-Aging Agents on TSC-STB Differentiation

First, a known anti-aging drug Rapamycin was tested. TSCs in good growing state were seeded at 1.0×10⁴-2×10⁴ cells per well in a 24-well plate pre-coated with 10 mg/mL Geltrex™ LDEV-Free Reduced Growth Factor Basement Membrane Matrix or Matrigel. After 24 hours, 3 wells continued in TSCM; 3 wells switched to STBM+DMSO; 3 wells switched to STBM+Rapamycin (500 nM). The media was changed every 2-3 days. On day 6, β-hCG was measured by ELISA as described in Example 1. Expression of aging-related marker genes (e.g., CGA and CGB3) was measured by RT-qPCR as described in Example 1.

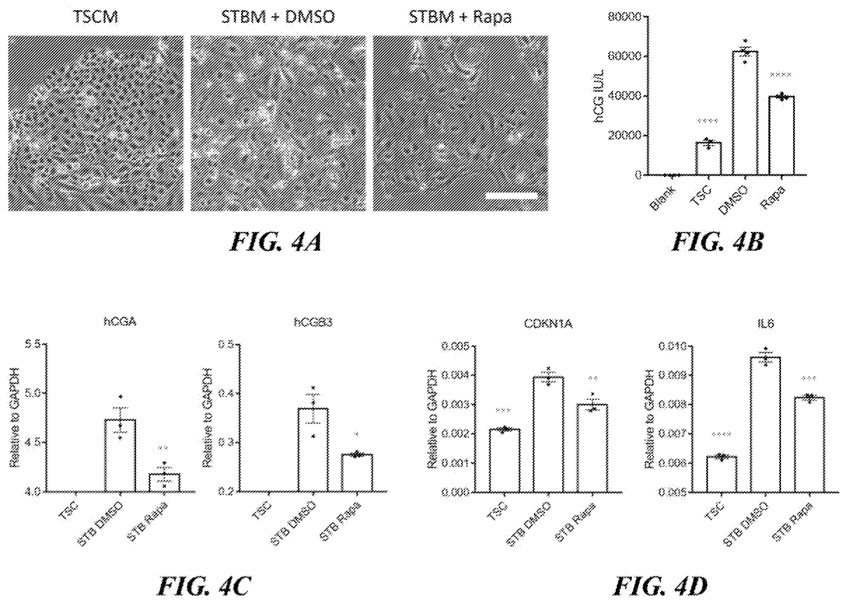

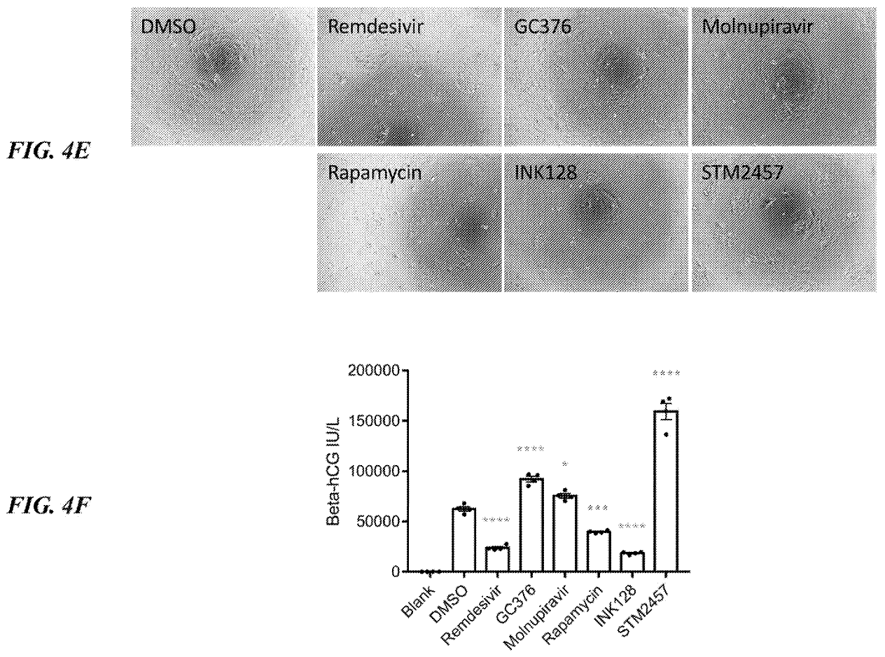

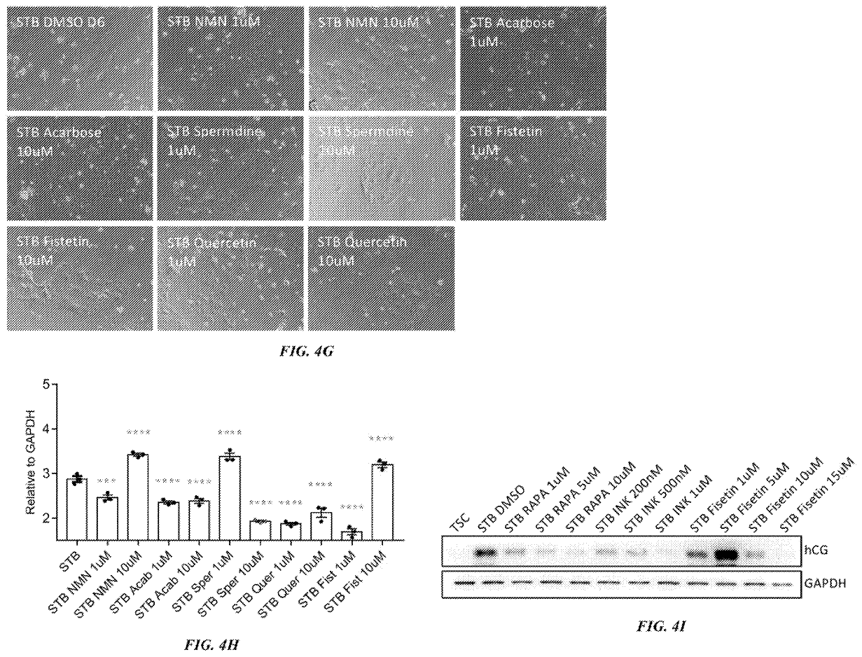

Known anti-ageing drug rapamycin decreased the STB features, SASP IL-6 and DNA-damage related gene CDKN1A expression level. Rapamycin at 500 nM did not affect cell viability or proliferation (FIG. 4A). Cells treated with Rapamycin showed significantly lower β-hCG, CGA, CGB3, CDKN1A and IL6 compared to the group in STBM+DMSO (FIGS. 4B-4D).

In addition to Rapamycin, several other drugs were also tested for potential anti-aging effects. FIG. 4E shows morphology of Remdesivir, GC376, Molnupiravir, Rapamycin, INK128, and STM2457 treated cells during TSC-STB differentiation, where delayed appearance of multinucleated STB cells was observed. ELISA result in FIG. 4F showed that cells treated with Remdesivir, Rapamycin, and INK128 which are known to have anti-aging effects secreted significantly lower level of β-hCG, suggesting that TSC-STB differentiation was inhibited or delayed. Cells treated with antiviral drugs Molnupiravir and GC376, and Mettle3 inhibitor STM2457 showed similar or increased levels of β-hCG compared to the DMSO control group.

The system was then used to test other agents. Nicotinamide mononucleotide (NMN), Acarbose, Spermdine, Fisetin, and Quercetin have been reported to have anti-aging effects. FIG. 4G shows morphology of Nicotinamide mononucleotide (NMN), Acarbose, Spermdine, Fisctin, and Quercetin treated cells during TSC-STB differentiation. NMN, Acarbose, and Fistetin reduced CGA expression at a lower concentration (1 μM) while increasing CGA expression at a higher concentration (10 μM), suggesting the further screening could be conducted at lower concentrations. On the other hand, Spermdine and Quercetin were shown to decrease CGA expression at both 1 μM and 10 μM, suggesting a need for further tests of more concentrations and that their anti-aging mechanism might be different from NMN, Acarbose, and Fistetin (FIG. 4H). Western blot showed that Rapamycin, INK128 and Fisctin at certain concentrations could significantly reduce β-hCG expression (FIG. 4I), further indicating the anti-aging effects of these agents.

### Example 5: Green Fluorescent Protein (GFP) Reporter Cell Line

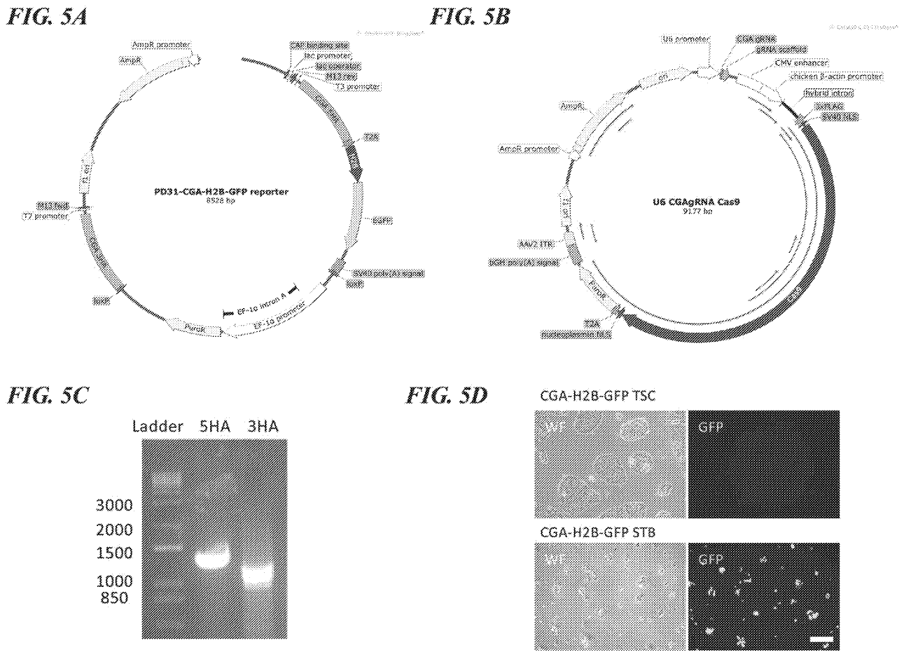

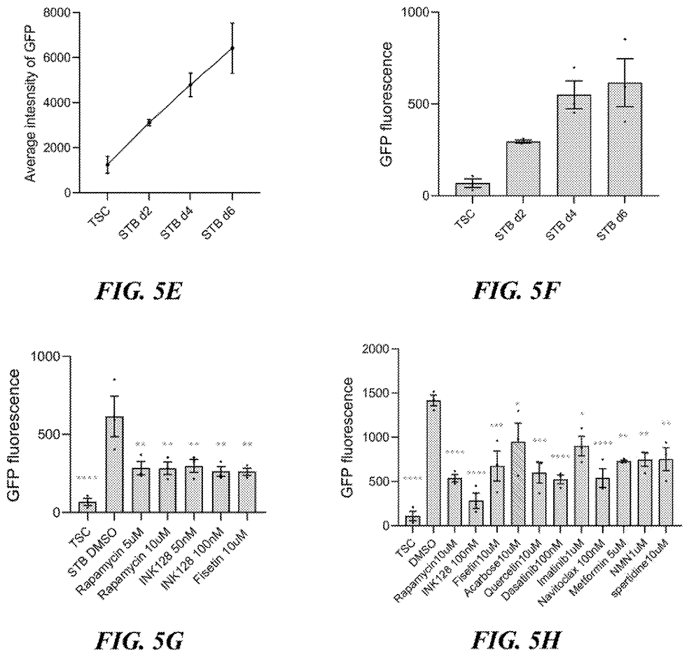

PD31-CGA-H2B-GFP and hSp-CGAgRNA-Cas9-2A-Puro (PX459) plasmids were constructed as described in Example 1 (FIGS. 5A-5B) and transfected into EPSC cells. After puromycin selection, colonies were picked and expanded and confirmed the insertion part of PD31-CGA-H2B-GFP at both 5HA and 3HA homology regions flanking through genotyping and sequencing. One colony example showed the right band size of both 5HA and 3HA homology regions flankings by electrophoresis (FIG. 5C). The EPSC colony was differentiated to TSC for a stable cell line which was negative for GFP signals under light microscopy, while after induction of STB differentiation, strong GFP signals were detected, accompanied by significantly elevated CGA expression as shown in FIGS. 5D-5F.

The reporter cells were then used to test various agents. The GFP signals decreased when treated with agents known to have anti-aging effects, confirming that GFP readout of the reporter cells can be used as an evaluation criterion for testing potential anti-aging agents (FIGS. 5G-5H).

In summary, the present invention established stable trophoblast stem/progenitor cells, which were successfully induced to differentiate into STBs. The present invention also characterized changes during TSC-STB differentiation using cell morphology, biomarkers, secretion functions (e.g., β-hCG), and bioinformatics, which can be used for evaluation of aging. The usefulness of TSC-STB differentiation as a system for evaluating aging was validated using known an anti-aging drug Rapamycin, where cells treated with Rapamycin showed slowed maturation and aging in both cell morphology and functions (e.g. β-hCG secretion), and aging-related biomarkers also showed significant changes. Other anti-aging compounds (e.g. metformin, NMN, Fisctin, and Quercetin) also showed anti-aging effects as expected in the TSC-STB system.

It will be appreciated that while the STBs in the above examples were derived from stem cells, they could also be derived from placental tissues.

It will be appreciated that while the cells in the above examples were human cells, mammal cells could also be used.

It will also be appreciated while the cells in the above examples were in a 2D culture. the cells could also be in a 3D culture.

SEQUENCES

| SEQ | | |
|---|---|---|
| ID | | |
| NO: | Annotation | Sequence (5′-3′) |
| SEQ | pCD31-5HA- | CTGACGCGCCCTGTAGCGGCGCATTAAGCGCGGCGGGTGTGGTGGGTATC |
| ID | T2A-H2B- | CGGTAAGCGGCAGGGTCGGAACAGGAGAGCGCACGAGGGAGCTTCCAGGG |
| NO: 1 | EGFP-loxp- | GGAAACGCCTGGTATCTTTATAGTCCTGTCGGGTTTCGCCACCTCTGACT |
| | EF1a-puro- | TGAGCGTCGATTTTTGTGATGCTCGTCAGGGGGGCGGAGCCTATGGAAAA |
| | loxp-3HA | ACGCCAGCAACGCGGCCTTTTTACGGTTCCTGGCCTTTTGCTGGCCTTTT |
| | | GCTCACATGTTCTTTCCTGCGTTATCCCCTGATTCTGTGGATAACCGTAT |
| | | TACCGCCTTTGAGTGAGCTGATACCGCTCGCCGCAGCCGAACGACCGAGC |
| | | GCAGCGAGTCAGTGAGCGAGGAAGCGGAAGAGCGCCCAATACGCAAACCG |
| | | CCTCTCCCCGCGCGTTGGCCGATTCATTAATGCAGCTGGCACGACAGGTT |
| | | TCCCGACTGGAAAGCGGGCAGTGAGCGCAACGCAATTAATGTGAGTTAGC |
| | | TCACTCATTAGGCACCCCAGGCTTTACACTTTATGCTTCCGGCTCGTATG |
| | | TTGTGTGGAATTGTGAGCGGATAACAATTTCACACAGGAAACAGCTATGA |
| | | CCATGATTACGCCAAGCTCGAAATTAACCCTCACTAAAGGGAACAAAAGC |
| | | TGGAGCTCCACCGCGGTGGCGGCCGCGCAGGACCAGATCAATACAGGGAC |
| | | TCCATCTCAGCCAGCCCTCAGGGAGACAGACAGCCCGGCCATGGGGTCCC |
| | | CAGACCTGATTATCCCCTCGACTTCTCCTGCTCTGACTTCCCAGAGTCCC |
| | | ACGTGCAGCGGGGTGCCATGTGCTCACAGAGGGCCCAGAGCCCTGGACTC |
| | | AGATGCTGCCTGCTCTTTGGGACTCACTCTTGCCCAGGGCCCCTCTACGC |
| | | CAGTATTTCCCAGCATGCTCCACTCTAGCCTCCTTGCTGCATCCATTCAT |
| | | CAAAATCCACCCACCTCACTGCCCGGCTAGCAACACCATAGCCATGGCAA |
| | | CGTGGGATCCGAGCCTCGTCTGCAACCTATACGGCAGCTTGTGGTCATGC |
| | | CGGATCCTTAACCCACTGAGCGAGGCCAGGGATTGAACCCACATCCTCAT |
| | | GGACGCTAGCTGGGTTCGTTAACCACTGAGCCACGACAGGAACTCCCCAG |
| | | TCCTTCATTTTTAAAATTTAAAAACAGACCTCGAAAGGGTTCACCTCATG |
| | | TTGGAGGCAGAGCTGGAGCAAGGAACCAGGTCCCTCTTAAGGTTACTAGC |
| | | CTTCCTCTGGAGTGCGCAGCGGGTGCCTGATGAAATGGGTTGCCAGCGCA |
| | | ATGTGCAGTGTGTCCGTGGGAAACAGAGGCTGCAGGCGCTACACAGCAAA |
| | | TGTGGAAAGAAGAGAGATGAGTGTGGGCTCTGCAGTTCCTTTGTGACGTG |
| | | CTCTCTGCTCTTTGCATATCTGTCCCCTTGGGCCAGGCTTCCATCCTGAA |
| | | GGGTGTGACCATAGTGTGTCTTGTTTCTTGTCACCGCAGAGAACGGAAGG |
| | | CTCCCTTGATGGGACTactagtGAGGGCAGAGGAAGTCTTCTAACATGCG |
| | | GTGACGTGGAGGAGAATCCCGGACCGGGGGGACCAGAGCCAGCGAAGTCT |
| | | GCTCCCGCCCCGAAAAAGGGCTCCAAGAAGGCGGTGACTAAGGCGCAGAA |
| | | GAAAGACGGCAAGAAGCGCAAGCGCAGCCGCAAGGAGAGCTATTCCATCT |
| | | ATGTGTACAAGGTTCTGAAGCAGGTCCACCCTGACACCGGCATTTCGTCC |
| | | AAGGCCATGGGCATCATGAATTCGTTTGTGAACGACATTTTCGAGCGCAT |
| | | CGCAGGTGAGGCTTCCCGCCTGGCGCATTACAACAAGCGCTCGACCATCA |
| | | CCTCCAGGGAGATCCAGACGGCCGTGCGCCTGCTGCTGCCTGGGGAGTTG |
| | | GCCAAGCACGCCGTGTCCGAGGGTACTAAGGCCGTCACCAAGTACACCAG |
| | | CGCTAAATCCACCatggtgagcaagggcgaggagctgttcaccggggggt |
| | | gcccatcctggtcgagctggacggcgacgtaaacggccacaagttcagcg |
| | | tgtccggcgagggcgagggcgatgccacctacggcaagctgaccctgaag |
| | | ttcatctgcaccaccggcaagctgcccgtgccctggcccaccctcgtgac |
| | | caccctgacctacggcgtgcagtgcttcagccgctaccccgaccacatga |
| | | agcagcacgacttcttcaagtccgccatgcccgaaggctacgtccaggag |
| | | cgcaccatcttcttcaaggacgacggcaactacaagacccgcgccgaggt |
| | | gaagttcgagggcgacaccctggtgaaccgcatcgagctgaagggcatcg |
| | | acttcaaggaggacggcaacatcctggggcacaagctggagtacaactac |
| | | aacagccacaacgtctatatcatggccgacaagcagaagaacggcatcaa |
| | | ggtgaacttcaagatccgccacaacatcgaggacggcagcgtgcagctcg |
| | | ccgaccactaccagcagaacacccccatcggcgacggccccgtgctgctg |
| | | cccgacaaccactacctgagcacccagtccgccctgagcaaagaccccaa |
| | | cgagaagcgcgatcacatggtcctgctggagttcgtgaccgccgccggga |
| | | tcactctcggcatggacgagctgtacaagtaaTTAATTAATGATCATAAT |
| | | CAGCCATATCACATCTGTAGAGGTTTTACTTGCTTTAAAAAACCTCCCAC |
| | | ACCTCCCCCTGAACCTGAAACATAAAATGAATGCAATTGTTGTTGTTAAC |
| | | TTGTTTATTGCAGCTTATAATGGTTACAATAAAGCAATAGCATCACAAAT |
| | | TTCACAAATAAAGCATTTTTTTCACTGCATTCTAGTTGTGGTTTGTCCAA |
| | | ACTCATCAATGTATCTTATCATGTCTGGATCCATAACTTCGTATAATGTA |
| | | TGCTATACGAAGTTATGAGTAATTCATACAAAAGGACTCGCCCCTGCCTT |
| | | GGGGAATCCCAGGGACCGTCGTTAAACTCCCACTAACGTAGAACCCAGAG |
| | | ATCGCTGCGTTCCCGCCCCCTCACCCGCCCGCTCTCGTCATCACTGAGGT |
| | | GGAGAAGAGCATGCGTGAGGCTCCGGTGCCCGTCAGTGGGCAGAGCGCAC |
| | | ATCGCCCACAGTCCCCGAGAAGTTGGGGGGAGGGGTCGGCAATTGAACCG |
| | | GTGCCTAGAGAAGGTGGCGCGGGGTAAACTGGGAAAGTGATGTCGTGTAC |
| | | TGGCTCCGCCTTTTTCCCGAGGGTGGGGGAGAACCGTATATAAGTGCAGT |
| | | AGTCGCCGTGAACGTTCTTTTTCGCAACGGGTTTGCCGCCAGAACACAGG |
| | | TAAGTGCCGTGTGTGGTTCCCGCGGGCCTGGCCTCTTTACGGGTTATGGC |
| | | CCTTGCGTGCCTTGAATTACTTCCACGCCCCTGGCTGCAGTACGTGATTC |
| | | TTGATCCCGAGCTTCGGGTTGGAAGTGGGTGGGAGAGTTCGAGGCCTTGC |
| | | GCTTAAGGAGCCCCTTCGCCTCGTGCTTGAGTTGAGGCCTGGCTTGGGCG |
| | | CTGGGGCCGCCGCGTGCGAATCTGGTGGCACCTTCGCGCCTGTCTCGCTG |
| | | CTTTCGATAAGTCTCTAGCCATTTAAAATTTTTGATGACCTGCTGCGACG |
| | | CTTTTTTTCTGGCAAGATAGTCTTGTAAATGCGGGCCAAGATCTGCACAC |
| | | TGGTATTTCGGTTTTTGGGGCCGCGGGCGGCGACGGGGCCCGTGCGTCCC |
| | | AGCGCACATGTTCGGCGAGGCGGGGCCTGCGAGCGCGGCCACCGAGAATC |
| | | GGACGGGGGTAGTCTCAAGCTGGCCGGCCTGCTCTGGTGCCTGGCCTCGC |
| | | GCCGCCGTGTATCGCCCCGCCCTGGGCGGCAAGGCTGGCCCGGTCGGCAC |
| | | CAGTTGCGTGAGCGGAAAGATGGCCGCTTCCCGGCCCTGCTGCAGGGAGC |
| | | TCAAAATGGAGGACGCGGCGCTCGGGAGAGCGGGCGGGTGAGTCACCCAC |
| | | ACAAAGGAAAAGGGCCTTTCCGTCCTCAGCCGTCGCTTCATGTGACTCCA |
| | | CGGAGTACCGGGCGCCGTCCAGGCACCTCGATTAGTTCTCGAGCTTTTGG |
| | | AGTACGTCGTCTTTAGGTTGGGGGGAGGGGTTTTATGCGATGGAGTTTCC |
| | | CCACACTGAGTGGGTGGAGACTGAAGTTAGGCCAGCTTGGCACTTGATGT |
| | | AATTCTCCTTGGAATTTGCCCTTTTTGAGTTTGGATCTTGGTTCATTCTC |
| | | AAGCCTCAGACAGTGGTTCAAAGTTTTTTTCTTCCATTTCAGGTGTCGTG |
| | | AGCTCGTGTCGAGCAGCTGAAGCTTACCATGACCGAGTACAAGCCCACGG |
| | | TGCGCCTCGCCACCCGCGACGACGTCCCCAGGGCCGTACGCACCCTCGCC |
| | | GCCGCGTTCGCCGACTACCCCGCCACGCGCCACACCGTCGATCCAGATCG |
| | | CCACATCGAGCGGGTCACCGAGCTGCAAGAACTCTTCCTCACGCGCGTCG |
| | | GGCTCGACATCGGCAAGGTGTGGGTCGCGGACGACGGCGCCGCGGTGGCG |
| | | GTCTGGACCACGCCGGAGAGCGTCGAAGCGGGGGCGGTGTTCGCCGAGAT |
| | | CGGCCCGCGCATGGCCGAGTTGAGCGGTTCCCGGCTGGCCGCGCAGCAAC |
| | | AGATGGAAGGCCTCCTGGCGCCGCACCGGCCCAAGGAGCCCGCGTGGTTC |
| | | CTGGCCACCGTCGGCGTCTCGCCCGACCACCAGGGCAAGGGTCTGGGCAG |
| | | CGCCGTCGTGCTCCCCGGAGTGGAGGCGGCCGAGCGCGCCGGGGTGCCCG |
| | | CCTTCCTGGAGACCTCCGCGCCCCGCAACCTCCCCTTCTACGAGCGGCTC |
| | | GGCTTCACCGTCACCGCCGACGTCGAGGTGCCCGAAGGACCGCGCACCTG |
| | | GTGCATGACCCGCAAGCCCGGTGCCTGACGCCCGCCCCACGACCCGCAGC |
| | | GCCCGACCGAAAGGAGCGCACGACCCCATGCATCGATGATATCAGATCCC |
| | | CGGGATGCAGAAATTGATGATCTATTAAACAATAAAGATGTCCACTAAAA |
| | | TGGAAGTTTTTCCTGTCATACTTTGTTAAGAAGGGTGAGAACAGAGTACC |
| | | TACATTTTGAATGGAAGGATTGGAGCTACGGGGGTGGGGGTGGGGTGGGA |
| | | TTAGATAAATGCNTGCTCTTTACTGAAGGCTCTTTACTATTGCTTTATGA |
| | | TAATGTTTCATAGTTGGATATCATAATTTAAACAAGCAAAACCAAATTAA |
| | | GGGCCAGCTCATTCCTCCCACTCATGATCTATAGATCTATAGATCTCTCG |
| | | TGGGATCATTGTTTTTCTCTTGATTCCCACTTTGTGGTTCTAAGTACTGT |
| | | GGTTTCCAAATGTGTCAGTTTCATAGCCTGAAGAACGAGATCAGCAGCCT |
| | | CTGTTCCACATACACTTCATTCTCAGTATTGTTTTGCCAAGTTCTAATTC |
| | | CATCAGAAGCTGGTCGAGATAACTTCGTATAATGTATGCTATACGAAGTT |
| | | ATggcgcgccATGGGACTTAGGACCGCAGAGCAGACGCTCCTCTCCGGGA |
| | | GGACACCGCATTCTGAGAAGAGCGGACAGTCCTTGTGTTCCAAGAGTTTT |
| | | GCGCACTTATTTATGAACCTGCCCTGCTCCCACTGAATGTAGCAGTTCCT |
| | | CAGGCCAAGTCACCACTTCTTCAATCCATCTTAGCTACATTTATGCTGGG |
| | | TATAAAGAGAGAACGGTGGGGCTGGTTAATTTCCCACCCGGCCCGCCTCC |
| | | CACCGCATTGGAGCATCCCTGGGAGTGGAGAGCAGCTGCCGGAGGTTCAG |
| | | AGTAAACTGGCCATTGGGACACTTTGAAACTTGAATATTTTGTCTTGTAC |
| | | AGAGATAAAGAACTTTTCCAAGTACCCTGATACACGGAAACCAGTATCTG |
| | | ATTTGTAGTCAATCACTTCTAGCGAATGGCCTGGCATAGGGGCTAGCTTT |
| | | TCTCTTTGCCTGTGGTCCTTCTGAGGTCTGTGGTTCTGGGCAGTCCTTGT |
| | | TTCTTGGAAGTACCGGGCATGGACTTGACAGCTGCACCCTGTCCCCTCTG |
| | | TGACCTCGCCTGCCACAAATGCGAAAACAGAGCTGCTCGGGCACAACCCC |
| | | TTTGAGATGCCGACTTGAAGACAAGGCTCCTTCTGGGCGATGCACCAAAT |
| | | TGAAAATGAAGTTGGACGAAGCACAGATGTTCTTAAGCTGTTTTTCTCTC |
| | | CCCTTTCTTTCTCTTTCTCTACCTGCTGACGGCTGAAGGATGGGAAGATG |
| | | GTAGCCTACAGCAAATACTTTTTTTAATAGAAAAAGGAAATGCATATTTT |
| | | TCTTACTAATTTTTAAAAATGTATTCCTTGCTAAAGGAATGGTCTCCCGA |
| | | GATCTTCCGACGTTTCTGATGGTGTCTTGAGTGGATGGACGGTGGGCTGC |
| | | AGTGGGATCGCCCCAGTGAGCACACCTAACCGAAATCACTTCCCCTGTCC |
| | | TCCTCGTCGACCTCGAGGGGGGGCCCGGTACCCAATTCGCCCTATAGTGA |
| | | GTCGTATTACAATTCACTGGCCGTCGTTTTACAACGTCGTGACTGGGAAA |
| | | ACCCTGGCGTTACCCAACTTAATCGCCTTGCAGCACATCCCCCTTTCGCC |
| | | AGCTGGCGTAATAGCGAAGAGGCCCGCACCGATCGCCCTTCCCAACAGTT |
| | | GCGCAGCCTGAATGGCGAATGGGACGCGCCCTGTAGCGGCGCATTAAGCG |
| | | CGGCGGGTGTGGTGGTTACGCGCAGCGTGACCGCTACACTTGCCAGCGCC |
| | | CTAGCGCCCGCTCCTTTCGCTTTCTTCCCTTCCTTTCTCGCCACGTTCGC |
| | | CGGCTTTCCCCGTCAAGCTCTAAATCGGGGGCTCCCTTTAGGGTTCCGAT |
| | | TTAGTGCTTTACGGCACCTCGACCCCAAAAAACTTGATTAGGGTGATGGT |
| | | TCACGTAGTGGGCCATCGCCCTGATAGACGGTTTTTCGCCCTTTGACGTT |
| | | GGAGTCCACGTTCTTTAATAGTGGACTCTTGTTCCAAACTGGAACAACAC |
| | | TCAACCCTATCTCGGTCTATTCTTTTGATTTATAAGGGATTTTGCCGATT |
| | | TCGGCCTATTGGTTAAAAAATGAGCTGATTTAACAAAAATTTAACGCGAA |
| | | TTTTAACAAAATATTAACGCTTACAATTTAGAGCTCACGCTGTAGGTATC |
| | | TCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTC |
| | | CAACCCGGTAAGACACGACTTATCGCCACTGGCAGCAGCCACTGGTAACA |
| | | GGATTAGCAGAGCGAGGTATGTAGGCGGTGCTACAGAGTTCTTGAAGTGG |
| | | TGGCCTAACTACGGCTACACTAGAAGGACAGTATTTGGTATCTGCGCTCT |
| | | GCTGAAGCCAGTTACCTTCGGAAAAAGAGTTGGTAGCTCTTGATCCGGCA |
| | | AACAAACCACCGCTGGTAGCGGTGGTTTTTTTGTTTGCAAGCAGCAGATT |
| | | ACGCGCAGAAAAAAAGGATCTCAAGAAGATCCTTTGATCTTTTCTACGGG |
| | | GTCTGACGCTCAGTGGAACGAAAACTCACGTTAAGGGATTTTGGTCATGA |
| | | GATTATCAAAAAGGATCTTCACCTAGATCCTTTTAAATTAAAAATGAAGT |
| | | TTTAAATCAATCTAAAGTATATATGAGTAAACTTGGTCTGACAGTTACCA |
| | | ATGCTTAATCAGTGAGGCACCTATCTCAGCGATCTGTCTATTTCGTTCAT |
| | | CCATAGTTGCCTGACTCCCCGTCGTGTAGATAACTACGATACGGGAGGGC |
| | | TTACCATCTGGCCCCAGTGCTGCAATGATACCGCGAGACCCACGCTCACC |
| | | GGCTCCAGATTTATCAGCAATAAACCAGCCAGCCGGAAGGGCCGAGCGCA |
| | | GAAGTGGTCCTGCAACTTTATCCGCCTCCATCCAGTCTATTAATTGTTGC |
| | | CGGGAAGCTAGAGTAAGTAGTTCGCCAGTTAATAGTTTGCGCAACGTTGT |
| | | TGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATG |
| | | TTGTGCAAAAAAGCGGTTAGCTCCTTCGGTCCTCCGATCGTTGTCAGAAG |
| | | TAAGTTGGCCGCAGTGTTATCACTCATGGTTATGGCAGCACTGCATAATT |
| | | CTCTTACTGTCATGCCATCCGTAAGATGCTTTTCTGTGACTGGTGAGTAC |
| | | TCAACCAAGTCATTCTGAGAATAGTGTATGCGGCGACCGAGTTGCTCTTG |
| | | CCCGGCGTCAATACGGGATAATACCGCGCCACATAGCAGAACTTTAAAAG |
| | | TGCTCATCATTGGAAAACGTTCTTCGGGGCGAAAACTCTCAAGGATCTTA |
| | | CCGCTGTTGAGATCCAGTTCGATGTAACCCACTCGTGCACCCAACTGATC |
| | | TTCAGCATCTTTTACTTTCACCAGCGTTTCTGGGTGAGCAAAAACAGGAA |
| | | GGCAAAATGCCGCAAAAAAGGGAATAAGGGCGACACGGAAATGTTGAATA |
| | | CTCATACTCTTCCTTTTTCAATATTATTGAAGCATTTATCAGGGTTATTG |
| | | TCTCATGAGCGGATACATATTTGAATGTATTTAGAAAAATAAACAAATAG |
| | | GGGTTCCGCGCACATTTCCCCGAAAAGTGCCAC |
| SEQ | hSpCas9-2A- | gagggcctatttcccatgattccttcatatttgcatatacgatacaaggc |
| ID | Puro | tgttagagagataattggaattaatttgactgtaaacacaaagatattag |
| NO: 2 | | tacaaaatacgtgacgtagaaagtaataatttcttgggtagtttgcagtt |
| | | ttaaaattatgttttaaaatggactatcatatgcttaccgtaacttgaaa |
| | | gtatttcgatttcttggctttatatatcttgtggaaaggacgaaacaccg |
| | | ggtcttcgagaagacctgttttagagctagaaatagcaagttaaaataag |
| | | gctagtccgttatcaacttgaaaaagtggcaccgagtcggtgcttttttg |
| | | ttttagagctagaaatagcaagttaaaataaggctagtccgtttttagcg |
| | | cgtgcgccaattctgcagacaaatggctctagaggtacccgttacataac |
| | | ttacggtaaatggcccgcctggctgaccgcccaacgacccccgcccattg |
| | | acgtcaatagtaacgccaatagggactttccattgacgtcaatgggtgga |
| | | gtatttacggtaaactgcccacttggcagtacatcaagtgtatcatatgc |
| | | caagtacgccccctattgacgtcaatgacggtaaatggcccgcctggcat |
| | | tgtgcccagtacatgaccttatgggactttcctacttggcagtacatcta |
| | | cgtattagtcatcgctattaccatggtcgaggtgagccccacgttctgct |
| | | tcactctccccatctcccccccctccccacccccaattttgtatttattt |
| | | attttttaattattttgtgcagcgatgggggcggggggggggggggggcg |
| | | cgcgccaggcggggcggggcggggcgaggggggggcggggcgaggcggag |
| | | aggtgcggcggcagccaatcagagcggcgcgctccgaaagtttcctttta |
| | | tggcgaggcggcggcggcggcggccctataaaaagcgaagcgcgcggggg |
| | | gggagtcgctgcgacgctgccttcgccccgtgccccgctccgccgccgcc |
| | | tcgcgccgcccgccccggctctgactgaccgcgttactcccacaggtgag |
| | | cgggcgggacggcccttctcctccgggctgtaattagctgagcaagaggt |
| | | aagggtttaagggatggttggttggtggggtattaatgtttaattacctg |
| | | gagcacctgcctgaaatcactttttttcaggttggaccggtgccaccatg |
| | | gactataaggaccacgacggagactacaaggatcatgatattgattacaa |
| | | agacgatgacgataagatggccccaaagaagaagcggaaggtcggtatcc |
| | | acggagtcccagcagccgacaagaagtacagcatcggcctggacatcggc |
| | | accaactctgtgggctgggccgtgatcaccgacgagtacaaggtgcccag |
| | | caagaaattcaaggtgctgggcaacaccgaccggcacagcatcaagaaga |
| | | acctgatcggagccctgctgttcgacagcggcgaaacagccgaggccacc |
| | | cggctgaagagaaccgccagaagaagatacaccagacggaagaaccggat |
| | | ctgctatctgcaagagatcttcagcaacgagatggccaaggtggacgaca |
| | | gcttcttccacagactggaagagtccttcctggtggaagaggataagaag |
| | | cacgagcggcaccccatcttcggcaacatcgtggacgaggtggcctacca |
| | | cgagaagtaccccaccatctaccacctgagaaagaaactggtggacagca |
| | | ccgacaaggccgacctgcggctgatctatctggccctggcccacatgatc |
| | | aagttccggggccacttcctgatcgagggcgacctgaaccccgacaacag |
| | | cgacgtggacaagctgttcatccagctggtgcagacctacaaccagctgt |
| | | tcgaggaaaaccccatcaacgccagcggcgtggacgccaaggccatcctg |
| | | tctgccagactgagcaagagcagacggctggaaaatctgatcgcccagct |
| | | gcccggcgagaagaagaatggcctgttcggaaacctgattgccctgagcc |
| | | tgggcctgacccccaacttcaagagcaacttcgacctggccgaggatgcc |
| | | aaactgcagctgagcaaggacacctacgacgacgacctggacaacctgct |
| | | ggcccagatcggcgaccagtacgccgacctgtttctggccgccaagaacc |
| | | tgtccgacgccatcctgctgagcgacatcctgagagtgaacaccgagatc |
| | | accaaggcccccctgagcgcctctatgatcaagagatacgacgagcacca |
| | | ccaggacctgaccctgctgaaagctctcgtgcggcagcagctgcctgaga |
| | | agtacaaagagattttcttcgaccagagcaagaacggctacgccggctac |
| | | attgacggcggagccagccaggaagagttctacaagttcatcaagcccat |
| | | cctggaaaagatggacggcaccgaggaactgctcgtgaagctgaacagag |
| | | aggacctgctgcggaagcagcggaccttcgacaacggcagcatcccccac |
| | | cagatccacctgggagagctgcacgccattctgcggcggcaggaagattt |
| | | ttacccattcctgaaggacaaccgggaaaagatcgagaagatcctgacct |
| | | tccgcatcccctactacgtgggccctctggccaggggaaacagcagattc |
| | | gcctggatgaccagaaagagcgaggaaaccatcaccccctggaacttcga |
| | | ggaagtggtggacaagggcgcttccgcccagagcttcatcgagcggatga |
| | | ccaacttcgataagaacctgcccaacgagaaggtgctgcccaagcacagc |
| | | ctgctgtacgagtacttcaccgtgtataacgagctgaccaaagtgaaata |
| | | cgtgaccgagggaatgagaaagcccgccttcctgagcggcgagcagaaaa |
| | | aggccatcgtggacctgctgttcaagaccaaccggaaagtgaccgtgaag |
| | | cagctgaaagaggactacttcaagaaaatcgagtgcttcgactccgtgga |
| | | aatctccggcgtggaagatcggttcaacgcctccctgggcacataccacg |
| | | atctgctgaaaattatcaaggacaaggacttcctggacaatgaggaaaac |
| | | gaggacattctggaagatatcgtgctgaccctgacactgtttgaggacag |
| | | agagatgatcgaggaacggctgaaaacctatgcccacctgttcgacgaca |
| | | aagtgatgaagcagctgaagcggcggagatacaccggctggggcaggctg |
| | | agccggaagctgatcaacggcatccgggacaagcagtccggcaagacaat |
| | | cctggatttcctgaagtccgacggcttcgccaacagaaacttcatgcagc |
| | | tgatccacgacgacagcctgacctttaaagaggacatccagaaagcccag |
| | | gtgtccggccagggcgatagcctgcacgagcacattgccaatctggccgg |
| | | cagccccgccattaagaagggcatcctgcagacagtgaaggtggtggacg |
| | | agctcgtgaaagtgatgggccggcacaagcccgagaacatcgtgatcgaa |
| | | atggccagagagaaccagaccacccagaagggacagaagaacagccgcga |
| | | gagaatgaagcggatcgaagagggcatcaaagagctgggcagccagatcc |
| | | tgaaagaacaccccgtggaaaacacccagctgcagaacgagaagctgtac |
| | | ctgtactacctgcagaatggggggatatgtacgtggaccaggaactggac |
| | | atcaaccggctgtccgactacgatgtggaccatatcgtgcctcagagctt |
| | | tctgaaggacgactccatcgacaacaaggtgctgaccagaagcgacaaga |
| | | accggggcaagagcgacaacgtgccctccgaagaggtcgtgaagaagatg |
| | | aagaactactggcggcagctgctgaacgccaagctgattacccagagaaa |
| | | gttcgacaatctgaccaaggccgagagaggcggcctgagcgaactggata |
| | | aggccggcttcatcaagagacagctggtggaaacccggcagatcacaaag |
| | | cacgtggcacagatcctggactcccggatgaacactaagtacgacgagaa |
| | | tgacaagctgatccgggaagtgaaagtgatcaccctgaagtccaagctgg |
| | | tgtccgatttccggaaggatttccagttttacaaagtgcgcgagatcaac |
| | | aactaccaccacgcccacgacgcctacctgaacgccgtcgtgggaaccgc |
| | | cctgatcaaaaagtaccctaagctggaaagcgagttcgtgtacggcgact |
| | | acaaggtgtacgacgtgcggaagatgatcgccaagagcgagcaggaaatc |
| | | ggcaaggctaccgccaagtacttcttctacagcaacatcatgaacttttt |
| | | caagaccgagattaccctggccaacggcgagatccggaagcggcctctga |
| | | tcgagacaaacggcgaaaccggggagatcgtgtgggataagggccgggat |
| | | tttgccaccgtgcggaaagtgctgagcatgccccaagtgaatatcgtgaa |
| | | aaagaccgaggtgcagacaggcggcttcagcaaagagtctatcctgccca |
| | | agaggaacagcgataagctgatcgccagaaagaaggactgggaccctaag |
| | | aagtacggcggcttcgacagccccaccgtggcctattctgtgctggtggt |
| | | ggccaaagtggaaaagggcaagtccaagaaactgaagagtgtgaaagagc |
| | | tgctggggatcaccatcatggaaagaagcagcttcgagaagaatcccatc |
| | | gactttctggaagccaagggctacaaagaagtgaaaaaggacctgatcat |
| | | caagctgcctaagtactccctgttcgagctggaaaacggccggaagagaa |
| | | tgctggcctctgccggcgaactgcagaagggaaacgaactggccctgccc |
| | | tccaaatatgtgaacttcctgtacctggccagccactatgagaagctgaa |
| | | gggctcccccgaggataatgagcagaaacagctgtttgtggaacagcaca |
| | | agcactacctggacgagatcatcgagcagatcagcgagttctccaagaga |
| | | gtgatcctggccgacgctaatctggacaaagtgctgtccgcctacaacaa |
| | | gcaccgggataagcccatcagagagcaggccgagaatatcatccacctgt |
| | | ttaccctgaccaatctgggagcccctgccgccttcaagtactttgacacc |
| | | accatcgaccggaagaggtacaccagcaccaaagaggtgctggacgccac |
| | | cctgatccaccagagcatcaccggcctgtacgagacacggatcgacctgt |
| | | ctcagctgggaggcgacaaaaggccggcggccacgaaaaaggccggccag |
| | | gcaaaaaagaaaaaggaattcggcagtggagagggcagaggaagtctgct |
| | | aacatgcggtgacgtcgaggagaatcctggcccaatgaccgagtacaagc |
| | | ccacggtgcgcctcgccacccgcgacgacgtccccagggccgtacgcacc |
| | | ctcgccgccgcgttcgccgactaccccgccacgcgccacaccgtcgatcc |
| | | ggaccgccacatcgagcgggtcaccgagctgcaagaactcttcctcacgc |
| | | gcgtcgggctcgacatcggcaaggtgtgggtcgcggacgacggcgccgcg |
| | | gtggcggtctggaccacgccggagagcgtcgaagcgggggcggtgttcgc |
| | | cgagatcggcccgcgcatggccgagttgagcggttcccggctggccgcgc |
| | | agcaacagatggaaggcctcctggcgccgcaccggcccaaggagcccgcg |
| | | tggttcctggccaccgtcggcgtctcgcccgaccaccagggcaagggtct |
| | | gggcagcgccgtcgtgctccccggagtggaggcggccgagcgcgccgggg |
| | | tgcccgccttcctggagacctccgcgccccacaacctccccttctacgag |
| | | cggctcggcttcaccgtcaccgccgacgtcgaggtgcccgaaggaccgcg |
| | | cacctggtgcatgacccgcaagcccggtgcctgagaattctaactagagc |
| | | tcgctgatcagcctcgactgtgccttctagttgccagccatctgttgttt |
| | | gcccctcccccgtgccttccttgaccctggaaggtgccactcccactgtc |
| | | ctttcctaataaaatgaggaaattgcatcgcattgtctgagtaggtgtca |
| | | ttctattctggggggtggggggggcaggacagcaagggggaggattggga |
| | | agagaatagcaggcatgctggggagcggccgcaggaacccctagtgatgg |
| | | agttggccactccctctctgcgcgctcgctcgctcactgaggccgggcga |
| | | ccaaaggtcgcccgacgcccgggctttgcccgggcggcctcagtgagcga |
| | | gcgagcgcgcagctgcctgcaggggcgcctgatgcggtattttctcctta |
| | | cgcatctgtgcggtatttcacaccgcatacgtcaaagcaaccatagtacg |
| | | cgccctgtagcggcgcattaagcgcggcgggtgtggtggttacgcgcagc |
| | | gtgaccgctacacttgccagcgccctagcgcccgctcctttcgctttctt |
| | | cccttcctttctcgccacgttcgccggctttccccgtcaagctctaaatc |
| | | gggggctccctttagggttccgatttagtgctttacggcacctcgacccc |
| | | aaaaaacttgatttgggtgatggttcacgtagtgggccatcgccctgata |
| | | gacggtttttcgccctttgacgttggagtccacgttctttaatagtggac |
| | | tcttgttccaaactggaacaacactcaaccctatctcgggctattctttt |
| | | gatttataagggattttgccgatttcggcctattggttaaaaaatgagct |
| | | gatttaacaaaaatttaacgcgaattttaacaaaatattaacgtttacaa |
| | | ttttatggtgcactctcagtacaatctgctctgatgccgcatagttaagc |
| | | cagccccgacacccgccaacacccgctgacgcgccctgacgggcttgtct |
| | | gctcccggcatccgcttacagacaagctgtgaccgtctccgggagctgca |
| | | tgtgtcagaggttttcaccgtcatcaccgaaacgcgcgagacgaaagggc |
| | | ctcgtgatacgcctatttttataggttaatgtcatgataataatggtttc |
| | | ttagacgtcaggtggcacttttcggggaaatgtgcgcggaacccctattt |
| | | gtttatttttctaaatacattcaaatatgtatccgctcatgagacaataa |
| | | ccctgataaatgcttcaataatattgaaaaaggaagagtatgagtattca |
| | | acatttccgtgtcgcccttattcccttttttgcggcattttgccttcctg |
| | | tttttgctcacccagaaacgctggtgaaagtaaaagatgctgaagatcag |
| | | ttgggtgcacgagtgggttacatcgaactggatctcaacagcggtaagat |
| | | ccttgagagttttcgccccgaagaacgttttccaatgatgagcactttta |
| | | aagttctgctatgtggcgcggtattatcccgtattgacgccgggcaagag |
| | | caactcggtcgccgcatacactattctcagaatgacttggttgagtactc |
| | | accagtcacagaaaagcatcttacggatggcatgacagtaagagaattat |
| | | gcagtgctgccataaccatgagtgataacactgcggccaacttacttctg |
| | | acaacgatcggaggaccgaaggagctaaccgcttttttgcacaacatggg |
| | | ggatcatgtaactcgccttgatcgttgggaaccggagctgaatgaagcca |
| | | taccaaacgacgagcgtgacaccacgatgcctgtagcaatggcaacaacg |
| | | ttgcgcaaactattaactggcgaactacttactctagcttcccggcaaca |
| | | attaatagactggatggaggcggataaagttgcaggaccacttctgcgct |
| | | cggcccttccggctggctggtttattgctgataaatctggagccggtgag |
| | | cgtggaagccgcggtatcattgcagcactggggccagatggtaagccctc |
| | | ccgtatcgtagttatctacacgacggggagtcaggcaactatggatgaac |
| | | gaaatagacagatcgctgagataggtgcctcactgattaagcattggtaa |
| | | ctgtcagaccaagtttactcatatatactttagattgatttaaaacttca |
| | | tttttaatttaaaaggatctaggtgaagatcctttttgataatctcatga |
| | | ccaaaatcccttaacgtgagttttcgttccactgagcgtcagaccccgta |
| | | gaaaagatcaaaggatcttcttgagatcctttttttctgcgcgtaatctg |
| | | ctgcttgcaaacaaaaaaaccaccgctaccagcggtggtttgtttgccgg |
| | | atcaagagctaccaactctttttccgaaggtaactggcttcagcagagcg |
| | | cagataccaaatactgtccttctagtgtagccgtagttaggccaccactt |
| | | caagaactctgtagcaccgcctacatacctcgctctgctaatcctgttac |
| | | cagtggctgctgccagtggcgataagtcgtgtcttaccgggttggactca |
| | | agacgatagttaccggataaggcgcagcggtcgggctgaacggggggttc |
| | | gtgcacacagcccagcttggagcgaacgacctacaccgaactgagatacc |
| | | tacagcgtgagctatgagaaagcgccacgcttcccgaagggagaaaggcg |
| | | gacaggtatccggtaagcggcagggtcggaacaggagagcgcacgaggga |
| | | gcttccagggggaaacgcctggtatctttatagtcctgtcgggtttcgcc |
| | | acctctgacttgagcgtcgatttttgtgatgctcgtcaggggggcggagc |
| | | ctatggaaaaacgccagcaacgcggcctttttacggttcctggccttttg |
| | | ctggccttttgctcacatgt |

1. A mammalian cellular aging evaluation system comprising a totipotent or pluripotent stem cell or an early extraembryonic cell, wherein the totipotent or pluripotent stem cell or early extraembryonic cell optionally comprises a reporter molecule or a heterologous nucleic acid encoding a reporter molecule that is indicative of senescence or differentiation of the totipotent or pluripotent stem cell or early extraembryonic cell towards a trophoblast or syncytiotrophoblast (“STB”).

2. The mammalian cellular aging evaluation system of claim 1, wherein the totipotent or pluripotent stem cell or early extraembryonic cell is naturally occurring.

3. The mammalian cellular aging evaluation system of claim 1, wherein the totipotent or pluripotent stem cell or early extraembryonic cell is genetically modified.

4. The mammalian cellular aging evaluation system of any one of claims 1-3, wherein the totipotent or pluripotent stem cell or early extraembryonic cell comprises a reporter molecule.

5. The mammalian cellular aging evaluation system of claim 4, wherein the reporter molecule is selected from the group consisting of: a physically activated molecule and a chemically activated molecule.

6. The mammalian cellular aging evaluation system of any one of claims 1-3, wherein the totipotent or pluripotent stem cell or early extraembryonic cell comprises the heterologous nucleic acid.

7. The mammalian cellular aging evaluation system of claim 6, wherein the heterologous nucleic acid is integrated into the genome of the totipotent or pluripotent stem cell or early extraembryonic cell.

8. The mammalian cellular aging evaluation system of claim 7, wherein the heterologous nucleic acid is under the control of a promoter of an endogenous biomarker gene encoding a biomarker indicative of senescence or differentiation of the totipotent or pluripotent stem cell or early extraembryonic cell towards a trophoblast or STB.

9. The mammalian cellular aging evaluation system of claim 8, wherein the heterologous nucleic acid is introduced into the totipotent or pluripotent stem cell or early extraembryonic cell by a gene editing tool.

10. The mammalian cellular aging evaluation system of any one of claims 6-9, wherein the reporter molecule is selected from the group consisting of Green Fluorescent Protein (GFP), Red Fluorescent Protein (RFP), Yellow Fluorescent Protein (YFP), mCherry, tdTomato, photoconvertible fluorescent proteins, bioluminescence, enzyme assay, antibody-based assays, chloramphenicol acetyltransferase, and biosensors.

11. The mammalian cellular aging evaluation system of any one of claims 1-10, wherein the system comprises an early extraembryonic cell.

12. The mammalian cellular aging evaluation system of claim 11, wherein the early extraembryonic cell is a trophoblast stem cell (“TSC”) or a trophoblast progenitor cell (“TPC”).

13. The mammalian cellular aging evaluation system of claim 12, wherein the early extraembryonic cell is a TSC.

14. The mammalian cellular aging evaluation system of any one of claims 11-13, wherein the early extraembryonic cell is derived from a totipotent stem cell, a pluripotent stem cell, an embryo tissue, or a placenta tissue.

15. The mammalian cellular aging evaluation system of any one of claims 11-14, wherein the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

16. The mammalian cellular aging evaluation system of any one of claims 8-15, wherein the endogenous biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion. and stem cell exhaustion.

17. The mammalian cellular aging evaluation system of claim 16, wherein the endogenous biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, and SIRT family members.

18. The mammalian cellular aging evaluation system of any one of claims 1-10, wherein the system comprises a totipotent or pluripotent stem cell.

19. The mammalian cellular aging evaluation system of claim 18, wherein the system comprises a totipotent stem cell.

20. The mammalian cellular aging evaluation system of claim 18, wherein the system comprises a pluripotent stem cell.

21. The mammalian cellular aging evaluation system of any one of claims 1-20, wherein the totipotent or pluripotent stem cell or early extraembryonic cell is derived from a human, a pig, a cow, a mouse, a rat, a bat, a rabbit, a dog, a cat, and a sheep.

22. A method of assessing an aging process of a mammalian cell, comprising subjecting a totipotent or pluripotent stem cell or an early extraembryonic cell to a condition that allows differentiation of the totipotent or pluripotent stem cell or extraembryonic cell towards a trophoblast or STB, and determining one or more characteristics of differentiation of the totipotent or pluripotent stem cell or the early extraembryonic cell.

23. A method of evaluating anti-aging function of a candidate agent, comprising:
1) subjecting a totipotent or pluripotent stem cell or an early extraembryonic cell to a condition that allows differentiation of the totipotent or pluripotent stem cell or early extraembryonic cell towards a trophoblast, specifically STB;
2) contacting the totipotent or pluripotent stem cell or early extraembryonic cell with the candidate agent before, during, or after subjecting the totipotent or pluripotent stem cell or early extraembryonic cell to a condition that allows the differentiation; and
3) assessing change of one or more characteristics of differentiation of the totipotent or pluripotent stem cell or early extraembryonic cell relative to a totipotent or pluripotent stem cell or an early extraembryonic cell without contacting with the candidate agent.

24. The method of claim 23, wherein evaluating anti-aging function of the candidate agent comprises assessing whether the candidate agent has anti-aging function.

25. The method of claim 23, wherein evaluating anti-aging function of the candidate agent comprises determining an effective concentration of the candidate agent having an anti-aging function.

26. A method of screening for a candidate agent having an anti-aging function, comprising:
1) evaluating the anti-aging function of a plurality of candidate agents according to the method of any one of claims 23-25; and
2) identifying the candidate agent having an anti-aging function based on the ability of the candidate agent to cause change of one or more characteristics of differentiation of the totipotent or pluripotent stem cell or early extraembryonic cell to a trophoblast or STB relative to a totipotent or pluripotent stem cell or early extraembryonic cell without the candidate agent.

27. The method of any one of claims 23-26, wherein the candidate agent is selected from the group consisting of: an antibody, a virus, a virus-like, a small molecule, a peptide, a polypeptide, a DNA, an mRNA, a guide RNA, a microRNA, an RNAi, a LncRNA, an siRNA molecule, and an antisense RNA.

28. The method of any one of claims 23-26, wherein the candidate agent is a naturally occurring substance.

29. The method of any one of claims 23-26, wherein the candidate agent is a nutraceutical.

30. A method of identifying a candidate gene involved in an aging process, comprising:
1) subjecting a plurality of totipotent or pluripotent stem cells or early extraembryonic cells to a condition that allows differentiation of the plurality of totipotent or pluripotent stem cells or early extraembryonic cells towards trophoblasts or STBs, wherein each of the plurality of totipotent or pluripotent stem cells or early extraembryonic cells comprises an alteration in a candidate gene relative to a wildtype totipotent or pluripotent stem cell or early extraembryonic cell, wherein at least two of the plurality of totipotent or pluripotent stem cells or early extraembryonic cells contain different alterations;
2) determining one or more characteristics of differentiation of the plurality of totipotent or pluripotent stem cells or early extraembryonic cells; and
3) identifying the candidate gene involved in an aging process based on the ability of the alteration in the candidate gene to cause change of one or more characteristics of differentiation of the plurality of totipotent or pluripotent stem cell or early extraembryonic cell relative to a totipotent or pluripotent stem cell or an early extraembryonic cell without the corresponding alternation.

31. The method of any one of claims 22-30, wherein the totipotent or pluripotent stem cell or early extraembryonic cell is naturally occurring.

32. The method of any one of claims 22-30, wherein the totipotent or pluripotent stem cell or early extraembryonic cell is genetically modified.

33. The method of any one of claims 22-32, wherein the totipotent or pluripotent stem cell or early extraembryonic cell comprises a reporter molecule that is indicative of differentiation of the totipotent or pluripotent stem cell or early extraembryonic cell towards an STB.

34. The method of claim 33, wherein the reporter molecule is selected from the group consisting of: a physically activated molecule and a chemically activated molecule.

35. The method of any one of claims 22-32, wherein the totipotent or pluripotent stem cell or early extraembryonic cell comprises a heterologous nucleic acid encoding a reporter molecule that is indicative of differentiation of the totipotent or pluripotent stem cell or early extraembryonic cell towards a trophoblast or STB.

36. The method of claim 35, wherein the heterologous nucleic acid is integrated into the genome of the totipotent or pluripotent stem cell or early extraembryonic cell.

37. The method of claim 36, wherein the heterologous nucleic acid is under the control of a promoter of an endogenous biomarker gene encoding a biomarker indicative of differentiation of the totipotent or pluripotent stem cell or early extraembryonic cell towards an STB.

38. The method of any one of claims 35-37, wherein the heterologous nucleic acid is introduced into the totipotent or pluripotent stem cell or early extraembryonic cell by a gene editing tool.

39. The method of any one of claims 35-38, wherein the reporter molecule is selected from the group consisting of Green Fluorescent Protein (GFP), Red Fluorescent Protein (RFP), Yellow Fluorescent Protein (YFP), mCherry, and tdTomato.

40. The method of any one of claims 22-39, wherein the STB is an early STB, a late STB, or a mature and aged STB.

41. The method of any one of claims 22-40, wherein the one or more characteristics of differentiation comprises:
1) presence or absence of a biomarker associated with differentiation of the totipotent or pluripotent stem cell or early extraembryonic cell;
2) level of a biomarker associated with differentiation of the totipotent or pluripotent stem cell or early extraembryonic cell;
3) secretion of a biomarker associated with differentiation of the totipotent or pluripotent stem cell or early extraembryonic cell;
4) cell morphology;
5) rate of change to a differentiated state;
6) property of a cellular organelle; and
7) number of nuclei in the cell; and
8) the presence or absence of a reporter molecule.

42. The method of claim 41, wherein the one or more characteristics of differentiation comprises property of a cellular organelle.

43. The method of claim 42, wherein the cellular organelle is selected from the group consisting of: mitochondria, proteosome, endoplasmic reticulum, Golgi apparatus, and nuclear envelope.

44. The method of claim 43, wherein the property of the cellular organelle comprises number, morphology, and function of the organelle.

45. The method of any one of claims 22-44, wherein the totipotent or pluripotent stem cell or early extraembryonic cell is an early extraembryonic cell.

46. The method of claim 45, wherein the early extraembryonic cell is derived from a stem cell selected from the group consisting of: an embryonic stem cell, an extraembryonic stem cell, an expanded potential stem cell (EPSC), a naive pluripotent stem cell, a primed pluripotent stem cell, an induced pluripotent stem cell, a 2-cell like cell, and an 8-cell-like cell.

47. The method of claim 41, wherein the one or more characteristics of differentiation comprises presence or absence of a biomarker associated with differentiation of the early extraembryonic cell.

48. The method of claim 47, wherein the biomarker is selected from the group consisting of a marker of genomic instability, epigenetic alternations (such as DNA, RNA and protein modifications), loss of proteostasis, telomere attrition, organelle dysfunction, disabled macroautophagy, deregulated nutrient-sensing, altered intercellular communication, cellular senescence, chronic inflammation, differentiation, endogenous transposon elements, cell cycle, and stem cell exhaustion, and stem cell exhaustion.

49. The method of claim 48, wherein the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, SIRT family members and combinations thereof.

50. The method of any one of claims 47-49, wherein the one or more characteristics of differentiation comprises molecular indicators of nucleotide excision repair (NER), base-excision repair (BER), DNA mismatch repair (MMR), Fanconi anemia pathway, homologous recombination (HR), nonhomologous end joining (NHEJ), variant histones, insulin resistance, pre-inflammation factors, the mTOR/AMPK pathway, mitophagy, senescence-associated secretory phenotype, and/or senescence.

51. The method of any one of claims 22-44, wherein the totipotent or pluripotent stem cell or early extraembryonic cell is a totipotent or pluripotent stem cell.

52. The method of claim 51, wherein the totipotent or pluripotent stem cell is a totipotent stem cell.

53. The method of claim 51, wherein the totipotent or pluripotent stem cell is a pluripotent stem cell.

54. The method of any one of claims 51-53, wherein the one or more characteristics of differentiation comprises presence or absence of a biomarker associated with differentiation of the totipotent or pluripotent stem cell.

55. The method of claim 54, wherein the biomarker is selected from the group consisting of β-hCG, β-galactosidase, NANOG, OCT4, SOX2, CDX2, GATA2, GATA3, KRT7, TEAD4, TFAP2C, TP53, CGA, CGB, ERVW1, CSH1, SDC1, HLA-G, MMP3, MMP9, ITGB6, GABRP, MUC16, IL1α, IL1β, IL6, IL8, IL28A, TIMP1, TIMP2, MCP1, MIP3, CXCL1, CXCL8, TGF, CCL2, BCL2, BCL2L1, BCL2L2, APEX1, MCL1, RB1, FOXO3, SMAD3, CDKN1A, CDKN1C, CDKN2A, CDKN2B, NF-κB, PTGS2, PTGES2, γH2AX, p21, p27, p38, p53, p57, PD1, PD-L1, Ki67, TFR1, METTL3, METTL14, WTAP, YTHDC1, YTHDF1, FTO, ALKBH5, ALKBH1, H3K9me3, H3K4me3, H3K27me3, H3K9ac3, SETDB1, KAP1, LaminA, LaminB1, LaminC, HP1γ, HP1α, LTR5, pTBK1, LINE1, HERVK, SIRT family members and combinations thereof.

56. The method of any one of claims 47-50 and 54-55, wherein the biomarker is a RNA molecule.

57. The method of claim 56, wherein assessing change of one or more characteristics of differentiation comprises RNA sequencing, RT-qPCR, and/or in situ hybridization.

58. The method of any one of claims 47-50 and 54-55, wherein the biomarker is a protein molecule.

59. The method of claim 58, wherein assessing change of one or more characteristics of differentiation comprises western blot, ELISA, proteomics, and/or immunofluorescence.

60. The method of any one of claims 23-29 and 31-59, wherein the totipotent or pluripotent stem cell or early extraembryonic cell is contacted with the candidate agent prior to being subjected to a condition for differentiation.

61. The method of any one of claims 23-29 and 31-59, wherein the totipotent or pluripotent stem cell or early extraembryonic cell is contacted with the candidate agent simultaneously with being subjected to a condition for differentiation.

62. The method of any one of claims 23-29 and 31-59, wherein the totipotent or pluripotent stem cell or early extraembryonic cell is contacted with the candidate agent after being subjected to a condition for differentiation.

63. The method of any one of claims 22-62, wherein the condition for differentiation comprises a cell culture medium comprising DMEM/F12, β-mercaptoethanol, Penicillin-Streptomycin-Glutamine, BSA, ITS-X, Y27632, Forskolin, and KnockOut Serum Replacement.

64. The method of any one of claims 22-63, wherein the characteristics of differentiation are evaluated at least one day after subjecting the totipotent or pluripotent stem cell or early extraembryonic cell to a condition for differentiation.

65. The method of claim 64, wherein the characteristics of differentiation are evaluated 2 to 8 days after subjecting the totipotent or pluripotent stem cell or early extraembryonic cell to a condition for differentiation.

66. The method of any one of claims 22-65, wherein the totipotent or pluripotent stem cell or early extraembryonic cell is derived from a human, a pig, a cow, a mouse, a rat, a bat, a rabbit, a dog, a cat, and a sheep.

67. The method of any one of claims 23-29 and 31-66, further comprising determining the effect of the candidate agent on the viability of the totipotent or pluripotent stem cell or early extraembryonic cell, or cells differentiated therefrom.
